(19) United States
(12) Patent Application Publication
(10) Pub. No.: US 2008/0306044 A1
(43) Pub. Date: Dec. 11, 2008

(54) SPIROBENZOAZEPANES AS VASOPRESSIN ANTAGONISTS

(76) Inventors: MICHAEL J. COSTANZO, Ivyland, PA (US); David F. McComsey, Warminster, PA (US); Stephen C. Yabut, Perkasie, PA (US); Han-Cheng Zhang, Lansdale, NJ (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 12/134,291

(22) Filed: Jun. 6, 2008

Related U.S. Application Data
(60) Provisional application No. 60/942,216, filed on Jun. 6, 2007.

(51) Int. Cl. A61K 31/55; C07D 223/14; A61P 9/10; A61P 9/12

(52) U.S. Cl. 514/212.02; 540/543

(57) ABSTRACT

The present invention is directed to a compound of Formula (I) or a form thereof:

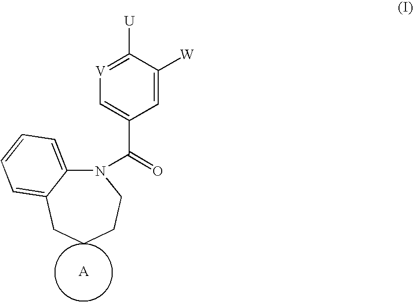

wherein U, V, W and Ring A are as defined herein, useful as vasopressin receptor antagonists.

### CROSS REFERENCE TO RELATED APPLICATION

This application claims the benefit of U.S. Provisional Application No. 60/942,216, filed Jun. 6, 2007, which is incorporated by reference herein.

### FIELD OF THE INVENTION

This invention is directed to substituted spirobenzoazepanes spiroheterocycles useful as vasopressin receptor antagonists. More particularly, the present invention provides methods of preparing such compounds and pharmaceutical compositions thereof and a method for treating a vasopressin receptor mediated condition using such compounds or pharmaceutical compositions.

### BACKGROUND OF THE INVENTION

Vasopressin is a nonapeptide hormone that is secreted primarily from the posterior pituitary gland. The hormone effects its actions through the vascular V-1 and renal V-2 receptor subtypes. The functions of vasopressin include contraction of uterine, bladder, and smooth muscle; stimulation of glycogen breakdown in the liver; induction of platelet aggregation; release of corticotropin from the anterior pituitary and stimulation of renal water reabsorption. As a neurotransmitter within the central nervous system (CNS), vasopressin can affect aggressive behavior, sexual behavior, the stress response, social behavior and memory. The V-1 a receptor mediates central nervous system effects, contraction of smooth muscle and hepatic glycogenolytic effects of vasopressin, while the V-1 b receptor mediates anterior pituitary effects of vasopressin. The V-2 receptor, presumably found only in the kidney, effects the antidiuretic actions of vasopressin via stimulation of adenylate cyclase (Liebsch, G et al Neurosci. 1996, 217, 101).

Elevated plasma vasopressin levels appear to play a role in the pathogenesis of congestive heart failure (P. A. Van Zwieten, Progr. Pharmacol. Clin. Pharmacol. 1990, 7, 49). As progress toward the treatment of congestive heart failure, nonpeptide vasopressin V-2 receptor antagonists have induced low osmolality aquaresis and decreased peripheral resistance in conscious dogs with congestive heart failure (H. Ogawa, J. Med. Chem. 1996, 39, 3547). In certain pathological states, plasma vasopressin levels may be inappropriately elevated for a given osmolality, thereby resulting in renal water retention and hyponatremia. Hyponatremia, associated with edematous conditions (cirrhosis, congestive heart failure, renal failure), can be accompanied by the syndrome of inappropriate secretion of antidiuretic hormone (SIADH). Treatment of SIADH-compromised rats with a vasopressin V-2 antagonist has corrected their existing hyponatremia (G. Fujisawa, Kidney Int. 1993, 44(1), 19). Due in part to the contractile actions of vasopressin at its V-1 receptor in the vasculature, vasopressin V-1 antagonists have reduced blood pressure as a potential treatment for hypertension as well. Known vasopressin receptor antagonists have included YM-087 (Yamanouchi); VPA-985, WAY-140288, and CL-385004 (American Home Products); SR-121463 (Sanofi-Synthelabo); and OPC 31260, OPC 41061, and OPC 21268 (Otsuka).

Thus, vasopressin receptor antagonists are useful in treating conditions such as edema, ischemia, inner ear disorders, hypertension, congestive heart failure, cardiac insufficiency, hyponatremia, coronary vasospasm, cardiac ischemia, liver cirrhosis, renal vasospasm, renal failure, polycystic kidney disease, diabetic nephropathy, cerebral edema and ischemia, stroke, thrombosis, and water retention. Additional conditions may include nephrotic syndrome, central nervous system injuries, dysmenorrhea, aggression, anxiety and obsessive-compulsive disorders.

United States Patent Application 20040266752 and PCT Publication WO 05/037795 describe substituted spiroheterocycles as vasopressin receptor antagonists and are incorporated herein by reference in their entirety and for all purposes.

### SUMMARY OF THE INVENTION

The present invention is directed to a compound of Formula (I) or a form thereof:

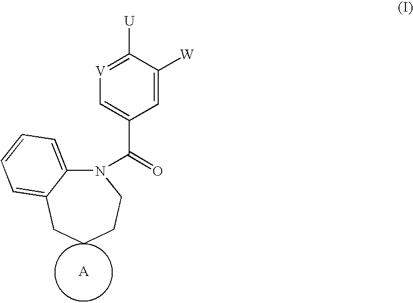

wherein U, V, W and Ring A are as defined herein, useful as vasopressin receptor antagonists.

The compounds of the present invention are vasopressin receptor antagonists which are useful in treating a vasopressin receptor mediated condition such as, but not limited hereto, edema, ischemia, inner ear disorders, hypertension, congestive heart failure, cardiac insufficiency, hyponatremia, coronary vasospasm, cardiac ischemia, liver cirrhosis, renal vasospasm, renal failure, polycystic kidney disease, diabetic nephropathy, cerebral edema and ischemia, stroke, thrombosis, water retention, aggression, obsessive-compulsive disorders, dysmenorrhea, nephrotic syndrome, anxiety and central nervous injuries.

The present invention also includes a pharmaceutical composition comprising a pharmaceutically acceptable carrier and any of the compounds of Formula (I) described above, and a pharmaceutical composition made by mixing one or more of the compounds of Formula (I) and a pharmaceutically acceptable carrier.

The invention also features a process for making a pharmaceutical composition comprising mixing any of the compounds described above and a pharmaceutically acceptable carrier.

The invention further provides methods for using a compound or composition of the invention. For example, one embodiment of the invention is a method for treating a condition associated with vasopressin receptor activity in a subject in need thereof comprising administering to the subject a effective amount of any of the disclosed compounds or the disclosed pharmaceutical compositions.

Other embodiments and features of the invention are disclosed in the following detailed description, examples, and the appended claims.

### DETAILED DESCRIPTION OF THE INVENTION

The present invention is directed to a compound of Formula (I):

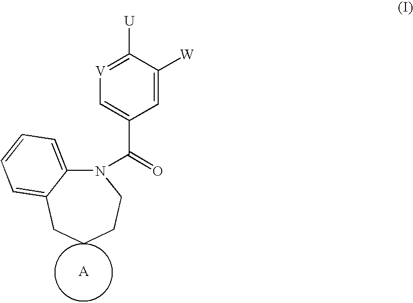

or a form thereof, wherein,
Ring A is selected from the group consisting Ring R₁a, Ring R₁b, Ring R₁c, Ring R₁d, Ring R₁e, Ring R₁f, Ring R₁g, Ring R₁h, Ring R₁i, Ring R₂a, Ring R₂b, Ring R₂c, Ring R₂d, Ring R₂e, Ring R₂f, Ring R₂g, Ring R₂h, and Ring R₂i, of the formulae:

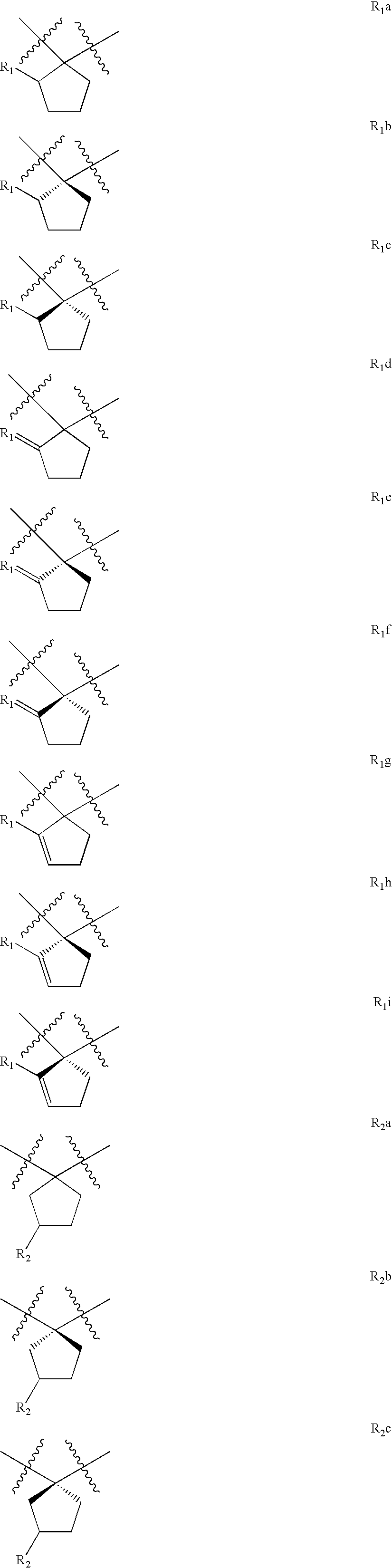

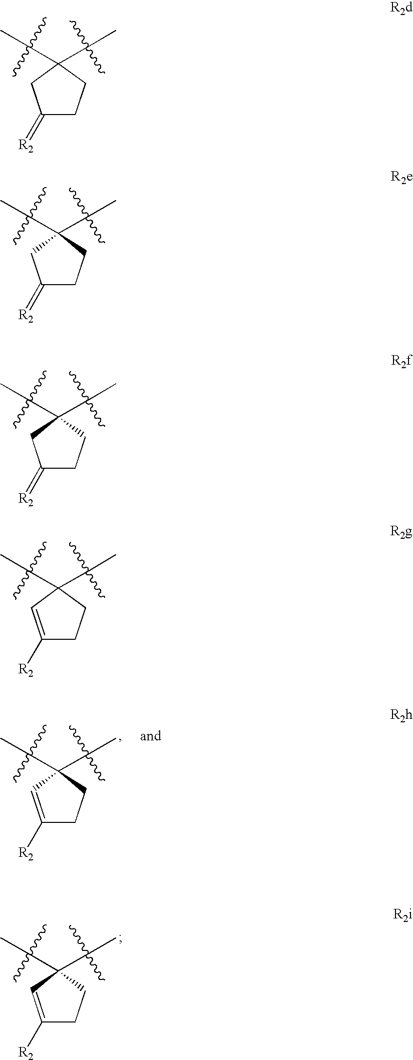

U is phenyl-carbonyl-amino, biphenyl-carbonyl-amino, heterocyclyl or heteroaryl,
wherein heterocyclyl and heteroaryl are each optionally substituted with C₁₋₄alkyl, and
wherein each phenyl is optionally substituted with one, two or three substituents independently selected from C₁₋₄alkyl, C₁₋₄alkoxy, halogen, hydroxy, carboxy, amino, C₁₋₄alkyl-amino or diC₁₋₄alkyl-amino;
V is CH or N;
W is hydrogen or C₁₋₃alkoxy;
R₁ is amino, C₁₋₄alkyl-amino, diC₁₋₄alkyl-amino, hydroxy-amino, amino-C₁₋₄alkyl-carbonyl-amino, C₁₋₄alkyl-amino-C₁₋₄alkyl-carbonyl-amino, diC₁₋₄alkyl-amino-C₁₋₄alkyl-carbonyl-amino, amino-sulfonyl-amino, C₁₋₄alkyl-imino, C₁₋₄alkoxy-imino, hydroxy-imino, amino-imino, C₁₋₄alkyl-amino-imino, diC₁₋₄alkyl-amino-imino, amino-C₁₋₄alkoxy-imino, C₁₋₄alkyl-amino-C₁₋₄alkoxy-imino, diC₁₋₄alkyl-amino-C₁₋₄alkoxy-imino, heteroaryl, heteroaryl-amino-imino or heteroaryl-carbonyl-amino-imino, wherein each heteroaryl is optionally substituted with C₁₋₄alkyl; and,
R₂ is oxo, amino, C₁₋₄alkyl-amino, diC₁₋₄alkyl-amino, hydroxy-amino, amino-C₁₋₄alkyl, C₁₋₄alkyl-amino-C₁₋₄alkyl, diC₁₋₄alkyl-amino-C₁₋₄alkyl, amino-C₁₋₄alkyl-amino, C₁₋₄alkyl-amino-C₁₋₄alkyl-amino, diC₁₋₄alkyl-amino-C₁₋₄alkyl-amino, amino-sulfonyl-amino, amino-sulfonyl-amino-carbonyl, C₁₋₄alkyl-amino-sulfonyl-amino-carbonyl, diC₁₋₄alkyl-amino-sulfonyl-amino-carbonyl, C₁₋₄alkoxy-carbonyl-methylene, carboxy-methylene, amino-carbonyl-methylene, C₁₋₄alkyl-amino-carbonyl-methylene, diC₁₋₄alkyl-amino-carbonyl-methylene, hydroxy-C₁₋₄alkyl-amino-carbonyl-methylene, amino-C₁₋₄alkyl-amino-carbonyl-methylene, C₁₋₄alkyl-amino-C₁₋₄alkyl-amino-carbonyl-methylene, diC₁₋₄alkyl-amino-C₁₋₄alkyl-amino-carbonyl-methylene, heterocyclyl-C₁-₄alkyl-amino-carbonyl-methylene, C₁₋₄alkyl-imino, C₁₋₄alkoxy-imino, hydroxy-imino, carboxy-C₁₋₄alkoxy-imino, amino-imino, C₁₋₄alkyl-amino-imino, diC₁₋₄alkyl-amino-imino, aryl-oxy-imino, heterocyclyl or heteroaryl, wherein heterocyclyl and heteroaryl are each optionally substituted with C₁₋₄alkyl.

An example of the compound of Formula (I) or a form thereof, is a compound wherein,
Ring A is selected from the group consisting Ring R₁a, Ring R₁b, Ring R₁c, Ring R₁d, Ring R₁e, Ring R₁f, Ring R₁g, Ring R₁h, Ring R₁i, Ring R₂a, Ring R₂b, Ring R₂c, Ring R₂d, Ring R₂e, Ring R₂f, Ring R₂g, Ring R₂h, and Ring R₂i;
U is phenyl-carbonyl-amino, biphenyl-carbonyl-amino, pyrrolidinyl or pyrazolyl, wherein pyrazolyl is optionally substituted with C₁₋₄alkyl, and wherein each phenyl is optionally substituted with one, two or three substituents independently selected from C₁₋₄alkyl, C₁₋₄alkoxy, halogen, hydroxy, carboxy, amino, C₁₋₄alkyl-amino or diC₁₋₄alkyl-amino;
V is CH or N;
W is hydrogen or C₁₋₃alkoxy;
R₁ is amino, C₁₋₄alkyl-amino, diC₁₋₄alkyl-amino, hydroxy-amino, amino-C₁₋₄alkyl-carbonyl-amino, C₁₋₄alkyl-amino-C₁₋₄alkyl-carbonyl-amino, diC₁₋₄alkyl-amino-C₁₋₄alkyl-carbonyl-amino, amino-sulfonyl-amino, C₁₋₄alkyl-imino, C₁₋₄alkoxy-imino, hydroxy-imino, amino-imino, C₁₋₄alkyl-amino-imino, diC₁₋₄alkyl-amino-imino, amino-C₁₋₄alkoxy-imino, C₁₋₄alkyl-amino-C₁₋₄alkoxy-imino, diC₁₋₄alkyl-amino-C₁₋₄alkoxy-imino, 1H-imidazolyl, pyridinyl-amino-imino or pyridinyl-carbonyl-amino-imino, wherein 1H-imidazolyl is optionally substituted with C₁₋₄alkyl; and,
R₂ is oxo, amino, C₁₋₄alkyl-amino, diC₁₋₄alkyl-amino, hydroxy-amino, amino-C₁₋₄alkyl, C₁₋₄alkyl-amino-C₁₋₄alkyl, diC₁₋₄alkyl-amino-C₁₋₄alkyl, amino-C₁₋₄alkyl-amino, C₁₋₄alkyl-amino-C₁₋₄alkyl-amino, diC₁₋₄alkyl-amino-C₁₋₄alkyl-amino, amino-sulfonyl-amino, amino-sulfonyl-amino-carbonyl, C₁₋₄alkyl-amino-sulfonyl-amino-carbonyl, diC₁₋₄alkyl-amino-sulfonyl-amino-carbonyl, C₁₋₄alkoxy-carbonyl-methylene, carboxy-methylene, amino-carbonyl-methylene, C₁₋₄alkyl-amino-carbonyl-methylene, diC₁₋₄alkyl-amino-carbonyl-methylene, hydroxy-C₁₋₄alkyl-amino-carbonyl-methylene, amino-C₁₋₄alkyl-amino-carbonyl-methylene, C₁₋₄alkyl-amino-C₁₋₄alkyl-amino-carbonyl-methylene, diC₁₋₄alkyl-amino-C₁₋₄alkyl-amino-carbonyl-methylene, morpholinyl-C₁₋₄alkyl-amino-carbonyl-methylene, C₁₋₄alkyl-imino, C₁₋₄alkoxy-imino, hydroxy-imino, carboxy-C₁₋₄alkoxy-imino, amino-imino, C₁₋₄alkyl-amino-imino, diC₁₋₄alkyl-amino-imino, aryl-oxy-imino, pyrrolidinyl, piperazinyl, 4,5-dihydro-1H-imidazolyl, morpholinyl, tetrazolyl or 1H-imidazolyl, wherein piperazinyl and heteroaryl are each optionally substituted with C₁₋₄alkyl.

An example of the compound of Formula (I) or a form thereof, is a compound wherein,
Ring A is selected from the group consisting Ring R₁a, Ring R₁d, Ring R₁e, Ring R₁f, Ring R₁g, Ring R₂b, Ring R₂e and Ring R₂h;
U is phenyl-carbonyl-amino, biphenyl-carbonyl-amino, heterocyclyl or heteroaryl, wherein heteroaryl is optionally substituted with C₁₋₄alkyl, and wherein each phenyl is optionally substituted with one or two substituents independently selected from C₁₋₄alkyl or halogen;
V is CH or N;
W is hydrogen or C₁₋₃alkoxy;
R₁ is amino, diC₁₋₄alkyl-amino, hydroxy-amino, amino-C₁₋₄alkyl-carbonyl-amino, amino-sulfonyl-amino, C₁₋₄alkoxy-imino, hydroxy-imino, amino-imino, diC₁₋₄alkyl-amino-imino, amino-C₁₋₄alkoxy-imino, heteroaryl, heteroaryl-amino-imino or heteroaryl-carbonyl-amino-imino, wherein each heteroaryl is optionally substituted with C₁₋₄alkyl; and,
R₂ is oxo, amino, diC₁₋₄alkyl-amino, hydroxy-amino, diC₁₋₄alkyl-amino-C₁₋₄alkyl-amino, amino-sulfonyl-amino, amino-sulfonyl-amino-carbonyl, diC₁₋₄alkyl-amino-sulfonyl-amino-carbonyl, C₁₋₄alkoxy-carbonyl-methylene, carboxy-methylene, amino-carbonyl-methylene, C₁₋₄alkyl-amino-carbonyl-methylene, hydroxy-C₁₋₄alkyl-amino-carbonyl-methylene, diC₁₋₄alkyl-amino-C₁₋₄alkyl-amino-carbonyl-methylene, heterocyclyl-C₁₋₄alkyl-amino-carbonyl-methylene, C₁₋₄alkoxy-imino, hydroxy-imino, carboxy-C₁₋₄alkoxy-imino, diC₁₋₄alkyl-amino-imino, heterocyclyl or heteroaryl, wherein heterocyclyl is optionally substituted with C₁₋₄alkyl.

An example of the compound of Formula (I) or a form thereof, is a compound wherein,
Ring A is selected from the group consisting Ring R₁a, Ring R₁d, Ring R₁e, Ring R₁f, Ring R₁g, Ring R₂b, Ring R₂e and Ring R₂h;
U is phenyl-carbonyl-amino, biphenyl-carbonyl-amino, pyrrolidinyl or pyrazolyl, wherein pyrazolyl is optionally substituted with C₁₋₄alkyl, and wherein each phenyl is optionally substituted with one or two substituents independently selected from C₁₋₄alkyl or halogen;
V is CH or N;
W is hydrogen or C₁₋₃alkoxy;
R₁ is amino, diC₁₋₄alkyl-amino, hydroxy-amino, amino-C₁₋₄alkyl-carbonyl-amino, amino-sulfonyl-amino, C₁₋₄alkoxy-imino, hydroxy-imino, amino-imino, diC₁₋₄alkyl-amino-imino, amino-C₁₋₄alkoxy-imino, 1H-imidazolyl, pyridinyl-amino-imino or pyridinyl-carbonyl-amino-imino, wherein 1H-imidazolyl is optionally substituted with C₁₋₄alkyl; and,
R₂ is oxo, amino, diC₁₋₄alkyl-amino, hydroxy-amino, diC₁₋₄alkyl-amino-C₁₋₄alkyl-amino, amino-sulfonyl-amino, amino-sulfonyl-amino-carbonyl, diC₁₋₄alkyl-amino-sulfonyl-amino-carbonyl, C₁₋₄alkoxy-carbonyl-methylene, carboxy-methylene, amino-carbonyl-methylene, C₁₋₄alkyl-amino-carbonyl-methylene, hydroxy-C₁₋₄alkyl-amino-carbonyl-methylene, diC₁₋₄alkyl-amino-C₁₋₄alkyl-amino-carbonyl-methylene, morpholinyl-C₁₋₄alkyl-amino-carbonyl-methylene, C₁₋₄alkoxy-imino, hydroxy-imino, carboxy-C₁₋₄alkoxy-imino, diC₁₋₄alkyl-amino-imino, pyrrolidinyl, piperazinyl, 4,5-dihydro-1H-imidazolyl, morpholinyl, tetrazolyl or 1H-imidazolyl, wherein piperazinyl is optionally substituted with C₁₋₄alkyl.

An example of the compound of Formula (I) or a form thereof, is a compound wherein,
Ring A is selected from the group consisting Ring R₁a, Ring R₁d, Ring R₁e, Ring R₁f, Ring R₁g, Ring R₂b, Ring R₂e and Ring R₂h;
U is phenyl-carbonyl-amino, biphenyl-carbonyl-amino, heterocyclyl or heteroaryl, wherein heteroaryl is optionally substituted with C₁₋₄alkyl, and wherein each phenyl is optionally substituted with one or two substituents independently selected from C₁₋₄alkyl or halogen;
V is CH or N;
W is hydrogen or C₁₋₃alkoxy;
R₁ is amino, diC₁₋₄alkyl-amino, hydroxy-amino, amino-C₁₋₄alkyl-carbonyl-amino, amino-sulfonyl-amino, C₁₋₄alkoxy-imino, hydroxy-imino, amino-imino, diC₁₋₄alkyl-amino-imino, amino-C₁₋₄alkoxy-imino, heteroaryl, heteroaryl-amino-imino or heteroaryl-carbonyl-amino-imino, wherein each heteroaryl is optionally substituted with C₁₋₄alkyl; and,
R₂ is diC₁₋₄alkyl-amino, diC₁₋₄alkyl-amino-C₁₋₄alkyl-amino, amino-sulfonyl-amino, amino-sulfonyl-amino-carbonyl, diC₁₋₄alkyl-amino-sulfonyl-amino-carbonyl, C₁₋₄alkoxy-carbonyl-methylene, carboxy-methylene, amino-carbonyl-methylene, C₁₋₄alkyl-amino-carbonyl-methylene, hydroxy-C₁₋₄alkyl-amino-carbonyl-methylene, diC₁₋₄alkyl-amino-C₁₋₄alkyl-amino-carbonyl-methylene, heterocyclyl-C₁₋₄alkyl-amino-carbonyl-methylene, C₁₋₄alkoxy-imino, hydroxy-imino, carboxy-C₁₋₄alkoxy-imino, diC₁₋₄alkyl-amino-imino, heterocyclyl or heteroaryl.

An example of the compound of Formula (I) or a form thereof, is a compound wherein,
Ring A is selected from the group consisting Ring R₁a, Ring R₁d, Ring R₁e, Ring R₁f, Ring R₁g, Ring R₂b, Ring R₂e and Ring R₂h;
U is phenyl-carbonyl-amino, biphenyl-carbonyl-amino, pyrrolidinyl or pyrazolyl, wherein pyrazolyl is optionally substituted with C₁₋₄alkyl, and wherein each phenyl is optionally substituted with one or two substituents independently selected from C₁₋₄alkyl or halogen;
V is CH or N;
W is hydrogen or C₁₋₃alkoxy;
R₁ is amino, diC₁₋₄alkyl-amino, hydroxy-amino, amino-C₁₋₄alkyl-carbonyl-amino, amino-sulfonyl-amino, C₁₋₄alkoxy-imino, hydroxy-imino, amino-imino, diC₁₋₄alkyl-amino-imino, amino-C₁₋₄alkoxy-imino, 1H-imidazolyl, pyridinyl-amino-imino or pyridinyl-carbonyl-amino-imino, wherein 1H-imidazolyl is optionally substituted with C₁₋₄alkyl; and,
R₂ is diC₁₋₄alkyl-amino, diC₁₋₄alkyl-amino-C₁₋₄alkyl-amino, amino-sulfonyl-amino, amino-sulfonyl-amino-carbonyl, diC₁₋₄alkyl-amino-sulfonyl-amino-carbonyl, C₁₋₄alkoxy-carbonyl-methylene, carboxy-methylene, amino-carbonyl-methylene, C₁₋₄alkyl-amino-carbonyl-methylene, hydroxy-C₁₋₄alkyl-amino-carbonyl-methylene, diC₁₋₄alkyl-amino-C₁₋₄alkyl-amino-carbonyl-methylene, morpholinyl-C₁₋₄alkyl-amino-carbonyl-methylene, C₁₋₄alkoxy-imino, hydroxy-imino, carboxy-C₁₋₄alkoxy-imino, diC₁₋₄alkyl-amino-imino4,5-dihydro-1H-imidazolyl, morpholinyl, tetrazolyl or 1H-imidazolyl.

An example of the compound of Formula (I) or a form thereof, is a compound wherein,
Ring A is selected from the group consisting Ring R₁a, Ring R₁d, Ring R₁e, Ring R₁g, Ring R₂b, Ring R₂e and Ring R₂h;
U is phenyl-carbonyl-amino or biphenyl-carbonyl-amino, wherein each phenyl is optionally substituted with one or two substituents independently selected from C₁₋₄alkyl or halogen;
V is CH or N;
W is hydrogen or C₁₋₃alkoxy;
R₁ is amino, diC₁₋₄alkyl-amino, hydroxy-amino, amino-C₁₋₄alkyl-carbonyl-amino, amino-sulfonyl-amino, C₁₋₄alkoxy-imino, hydroxy-imino, amino-imino, diC₁₋₄alkyl-amino-imino, amino-C₁₋₄alkoxy-imino, heteroaryl, heteroaryl-amino-imino or heteroaryl-carbonyl-amino-imino, wherein each heteroaryl is optionally substituted with C₁₋₄alkyl; and,
R₂ is amino-sulfonyl-amino, amino-sulfonyl-amino-carbonyl, C₁₋₄alkoxy-carbonyl-methylene, carboxy-methylene, amino-carbonyl-methylene, C₁₋₄alkyl-amino-carbonyl-methylene, hydroxy-C₁₋₄alkyl-amino-carbonyl-methylene, hydroxy-imino or heteroaryl.

An example of the compound of Formula (I) or a form thereof, is a compound wherein,
Ring A is selected from the group consisting Ring R₁a, Ring R₁d, Ring R₁e, Ring R₁g, Ring R₂b, Ring R₂e and Ring R₂h;
U is phenyl-carbonyl-amino or biphenyl-carbonyl-amino, wherein each phenyl is optionally substituted with one or two substituents independently selected from C₁₋₄alkyl or halogen;
V is CH or N;
W is hydrogen or C₁₋₃alkoxy;
R₁ is amino, diC₁₋₄alkyl-amino, hydroxy-amino, amino-C₁₋₄alkyl-carbonyl-amino, amino-sulfonyl-amino, C₁₋₄alkoxy-imino, hydroxy-imino, amino-imino, diC₁₋₄alkyl-amino-imino, amino-C₁₋₄alkoxy-imino, 1H-imidazolyl, pyridinyl-amino-imino or pyridinyl-carbonyl-amino-imino, wherein 1H-imidazolyl is optionally substituted with C₁₋₄alkyl; and,
R₂ is amino-sulfonyl-amino, amino-sulfonyl-amino-carbonyl, C₁₋₄alkoxy-carbonyl-methylene, carboxy-methylene, amino-carbonyl-methylene, C₁₋₄alkyl-amino-carbonyl-methylene, hydroxy-C₁₋₄alkyl-amino-carbonyl-methylene, hydroxy-imino, tetrazolyl or 1H-imidazolyl.

An example of the compound of Formula (I) or a form thereof, is a compound wherein,
Ring A is selected from the group consisting Ring R₁d, Ring R₁e and Ring R₁g;
U is phenyl-carbonyl-amino, wherein phenyl is substituted with one or two halogen substituents;
V is CH;
W is hydrogen; and,
R₁ is C₁₋₄alkoxy-imino, hydroxy-imino, amino-imino, diC₁₋₄alkyl-amino-imino, amino-C₁₋₄alkoxy-imino, heteroaryl, or heteroaryl-amino-imino, wherein each heteroaryl is optionally substituted with C₁₋₄alkyl.

An example of the compound of Formula (I) or a form thereof, is a compound wherein,
Ring A is selected from the group consisting Ring R₁d, Ring R₁e and Ring R₁g;
U is phenyl-carbonyl-amino, wherein phenyl is substituted with one or two halogen substituents;
V is CH;
W is hydrogen; and,
R₁ is C₁₋₄alkoxy-imino, hydroxy-imino, amino-imino, diC₁₋₄alkyl-amino-imino, amino-C₁₋₄alkoxy-imino, 1H-imidazolyl or pyridinyl-amino-imino, wherein 1H-imidazolyl is optionally substituted with C₁₋₄alkyl.

An example of the compound of Formula (I) or a form thereof, is a compound wherein, Ring A is selected from the group consisting Ring R₁a, Ring R₁d, Ring R₁e, Ring R₁f, Ring R₁g, Ring R₂b, Ring R₂e and Ring R₂h.

An example of the compound of Formula (I) or a form thereof, is a compound wherein, Ring A is selected from the group consisting Ring R₁a, Ring R₁d, Ring R₁e, Ring R₁g, Ring R₂b, Ring R₂e and Ring R₂h.

An example of the compound of Formula (I) or a form thereof, is a compound wherein, Ring A is selected from the group consisting Ring R₁d, Ring R₁e and Ring R₁g.

An example of the compound of Formula (I) or a form thereof, is a compound wherein, U is phenyl-carbonyl-amino, biphenyl-carbonyl-amino, heterocyclyl or heteroaryl, wherein heteroaryl is optionally substituted with C₁₋₄alkyl, and wherein each phenyl is optionally substituted with one or two substituents independently selected from C₁₋₄alkyl or halogen.

An example of the compound of Formula (I) or a form thereof, is a compound wherein, U is phenyl-carbonyl-amino, biphenyl-carbonyl-amino, pyrrolidinyl or pyrazolyl, wherein pyrazolyl is optionally substituted with C₁₋₄alkyl, and wherein each phenyl is optionally substituted with one or two substituents independently selected from C₁₋₄alkyl or halogen.

An example of the compound of Formula (I) or a form thereof, is a compound wherein, U is phenyl-carbonyl-amino or biphenyl-carbonyl-amino, and wherein each phenyl is optionally substituted with one or two substituents independently selected from C₁₋₄alkyl or halogen.

An example of the compound of Formula (I) or a form thereof, is a compound wherein, U is phenyl-carbonyl-amino, and wherein phenyl is substituted with one or two halogen substituents.

An example of the compound of Formula (I) or a form thereof, is a compound wherein, V is CH.

An example of the compound of Formula (I) or a form thereof, is a compound wherein, W is hydrogen.

An example of the compound of Formula (I) or a form thereof, is a compound wherein, R₁ is amino, diC₁₋₄alkyl-amino, hydroxy-amino, amino-C₁₋₄alkyl-carbonyl-amino, amino-sulfonyl-amino, C₁₋₄alkoxy-imino, hydroxy-imino, amino-imino, diC₁₋₄alkyl-amino-imino, amino-C₁₋₄alkoxy-imino, heteroaryl, heteroaryl-amino-imino or heteroaryl-carbonyl-amino-imino, wherein each heteroaryl is optionally substituted with C₁₋₄alkyl.

An example of the compound of Formula (I) or a form thereof, is a compound wherein, R₁ is amino, diC₁₋₄alkyl-amino, hydroxy-amino, amino-C₁₋₄alkyl-carbonyl-amino, amino-sulfonyl-amino, C₁₋₄alkoxy-imino, hydroxy-imino, amino-imino, diC₁₋₄alkyl-amino-imino, amino-C₁₋₄alkoxy-imino, 1H-imidazolyl, pyridinyl-amino-imino or pyridinyl-carbonyl-amino-imino, wherein 1H-imidazolyl is optionally substituted with C₁₋₄alkyl.

An example of the compound of Formula (I) or a form thereof, is a compound wherein, R₁ is C₁₋₄alkoxy-imino, hydroxy-imino, amino-imino, diC₁₋₄alkyl-amino-imino, amino-C₁₋₄alkoxy-imino, heteroaryl, or heteroaryl-amino-imino, wherein each heteroaryl is optionally substituted with C₁₋₄alkyl.

An example of the compound of Formula (I) or a form thereof, is a compound wherein, R₁ is C₁₋₄alkoxy-imino, hydroxy-imino, amino-imino, diC₁₋₄alkyl-amino-imino, amino-C₁₋₄alkoxy-imino, 1H-imidazolyl or pyridinyl-amino-imino, wherein 1H-imidazolyl is optionally substituted with C₁₋₄alkyl.

An example of the compound of Formula (I) or a form thereof, is a compound wherein, R₂ is oxo, amino, diC₁₋₄alkyl-amino, hydroxy-amino, diC₁₋₄alkyl-amino-C₁₋₄alkyl-amino, amino-sulfonyl-amino, amino-sulfonyl-amino-carbonyl, diC₁₋₄alkyl-amino-sulfonyl-amino-carbonyl, C₁₋₄alkoxy-carbonyl-methylene, carboxy-methylene, amino-carbonyl-methylene, C₁₋₄alkyl-amino-carbonyl-methylene, hydroxy-C₁₋₄alkyl-amino-carbonyl-methylene, diC₁₋₄alkyl-amino-C₁₋₄alkyl-amino-carbonyl-methylene, heterocyclyl-C₁-₄alkyl-amino-carbonyl-methylene, C₁₋₄alkoxy-imino, hydroxy-imino, carboxy-C₁₋₄alkoxy-imino, diC₁₋₄alkyl-amino-imino, heterocyclyl or heteroaryl, wherein heterocyclyl is optionally substituted with C₁₋₄alkyl.

An example of the compound of Formula (I) or a form thereof, is a compound wherein, R₂ is oxo, amino, diC₁₋₄alkyl-amino, hydroxy-amino, diC₁₋₄alkyl-amino-C₁₋₄alkyl-amino, amino-sulfonyl-amino, amino-sulfonyl-amino-carbonyl, diC₁₋₄alkyl-amino-sulfonyl-amino-carbonyl, C₁₋₄alkoxy-carbonyl-methylene, carboxy-methylene, amino-carbonyl-methylene, C₁₋₄alkyl-amino-carbonyl-methylene, hydroxy-C₁₋₄alkyl-amino-carbonyl-methylene, diC₁₋₄alkyl-amino-C₁₋₄alkyl-amino-carbonyl-methylene, morpholinyl-C₁₋₄alkyl-amino-carbonyl-methylene, C₁₋₄alkoxy-imino, hydroxy-imino, carboxy-C₁₋₄alkoxy-imino, diC₁₋₄alkyl-amino-imino, pyrrolidinyl, piperazinyl, 4,5-dihydro-1H-imidazolyl, morpholinyl, tetrazolyl or 1H-imidazolyl, wherein piperazinyl is optionally substituted with C₁₋₄alkyl.

An example of the compound of Formula (I) or a form thereof, is a compound wherein, R₂ is diC₁₋₄alkyl-amino, diC₁₋₄alkyl-amino-C₁₋₄alkyl-amino, amino-sulfonyl-amino, amino-sulfonyl-amino-carbonyl, diC₁₋₄alkyl-amino-sulfonyl-amino-carbonyl, C₁₋₄alkoxy-carbonyl-methylene, carboxy-methylene, amino-carbonyl-methylene, C₁₋₄alkyl-amino-carbonyl-methylene, hydroxy-C₁₋₄alkyl-amino-carbonyl-methylene, diC₁₋₄alkyl-amino-C₁₋₄alkyl-amino-carbonyl-methylene, heterocyclyl-C₁₋₄alkyl-amino-carbonyl-methylene, C₁₋₄alkoxy-imino, hydroxy-imino, carboxy-C₁₋₄alkoxy-imino, diC₁₋₄alkyl-amino-imino, heterocyclyl or heteroaryl.

An example of the compound of Formula (I) or a form thereof, is a compound wherein, R₂ is diC₁₋₄alkyl-amino, diC₁₋₄alkyl-amino-C₁₋₄alkyl-amino, amino-sulfonyl-amino, amino-sulfonyl-amino-carbonyl, diC₁₋₄alkyl-amino-sulfonyl-amino-carbonyl, C₁₋₄alkoxy-carbonyl-methylene, carboxy-methylene, amino-carbonyl-methylene, C₁₋₄alkyl-amino-carbonyl-methylene, hydroxy-C₁₋₄alkyl-amino-carbonyl-methylene, diC₁₋₄alkyl-amino-C₁₋₄alkyl-amino-carbonyl-methylene, heterocyclyl-C₁₋₄alkyl-amino-carbonyl-methylene, C₁₋₄alkoxy-imino, hydroxy-imino, carboxy-C₁₋₄alkoxy-imino, diC₁₋₄alkyl-amino-imino, 4,5-dihydro-1H-imidazolyl, morpholinyl, tetrazolyl or 1H-imidazolyl.

An example of the compound of Formula (I) or a form thereof, is a compound wherein, R₂ is amino-sulfonyl-amino, amino-sulfonyl-amino-carbonyl, C₁₋₄alkoxy-carbonyl-methylene, carboxy-methylene, amino-carbonyl-methylene, C₁₋₄alkyl-amino-carbonyl-methylene, hydroxy-C₁₋₄alkyl-amino-carbonyl-methylene, hydroxy-imino or heteroaryl.

An example of the compound of Formula (I) or a form thereof, is a compound wherein, R₂ is amino-sulfonyl-amino, amino-sulfonyl-amino-carbonyl, C₁₋₄alkoxy-carbonyl-methylene, carboxy-methylene, amino-carbonyl-methylene, C₁₋₄alkyl-amino-carbonyl-methylene, hydroxy-C₁₋₄alkyl-amino-carbonyl-methylene, hydroxy-imino, tetrazolyl or 1H-imidazolyl.

Examples of a compound of Formula (I) include compounds selected from the group consisting of:

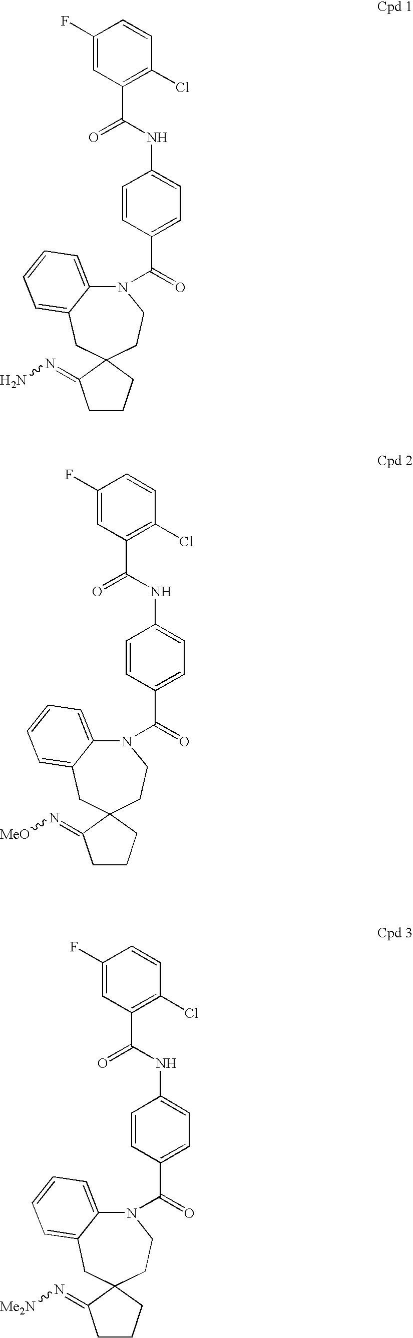

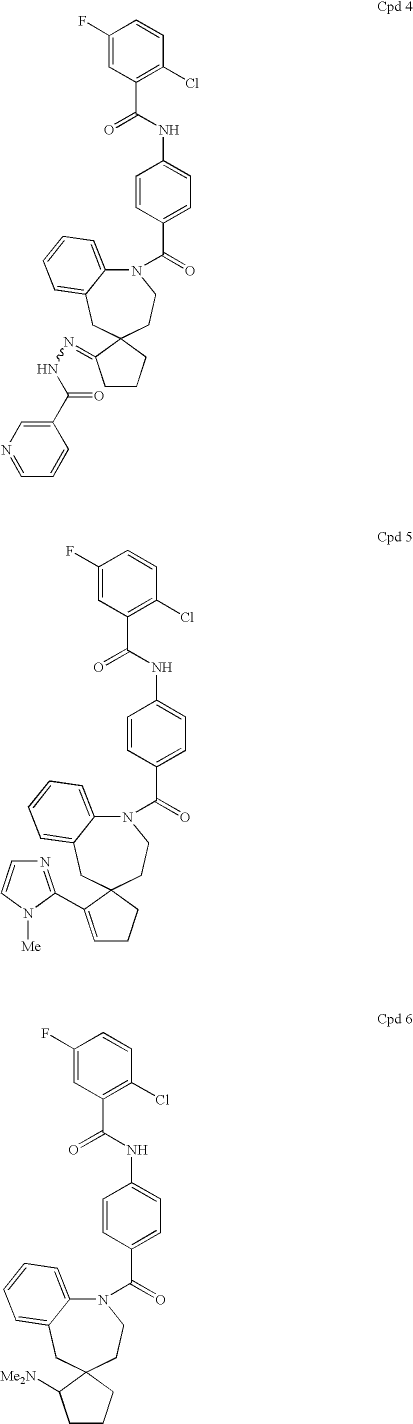

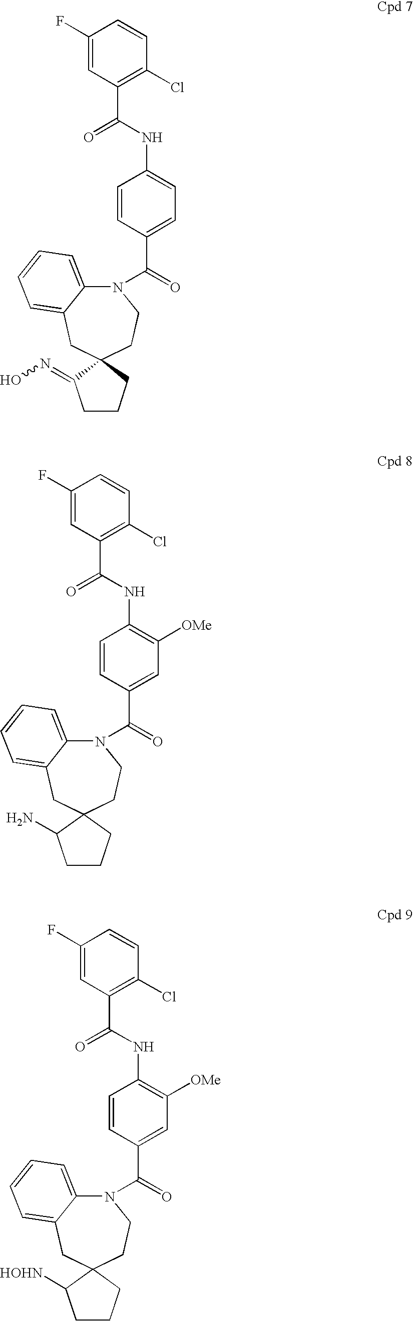

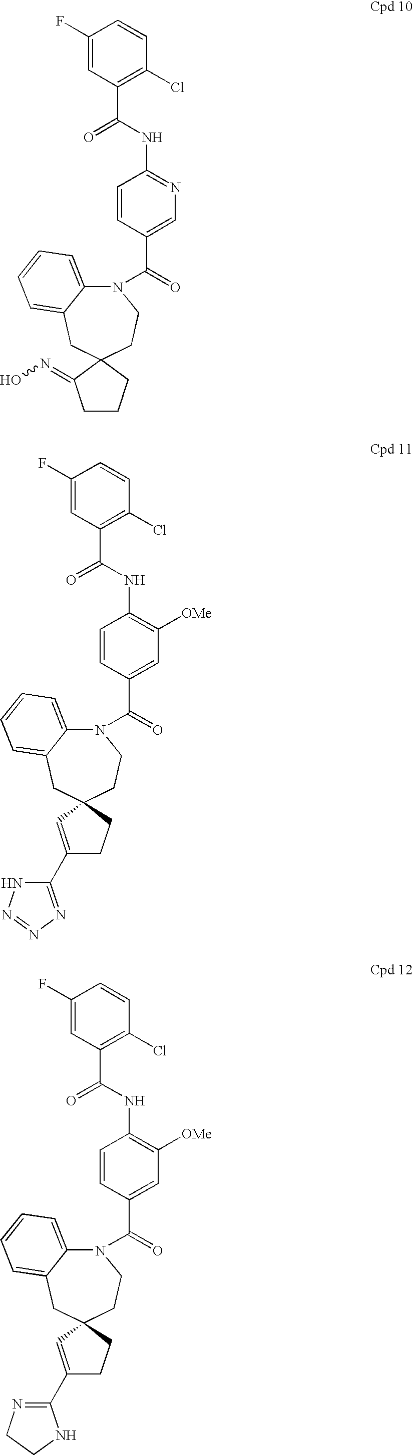

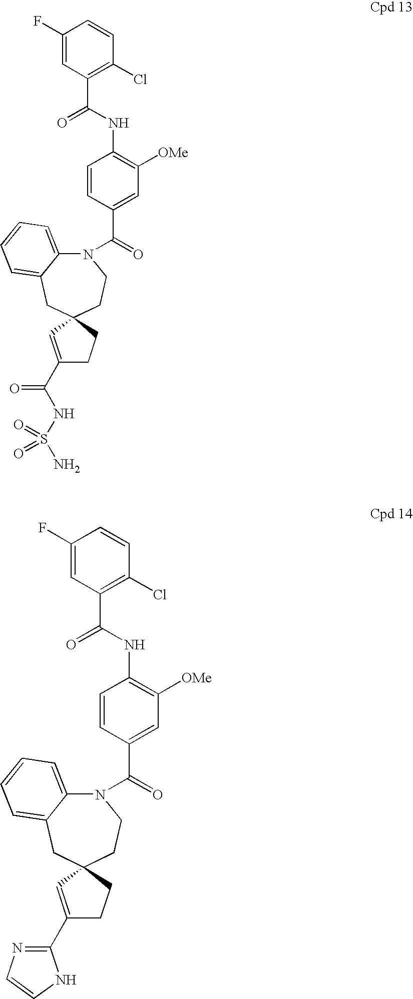

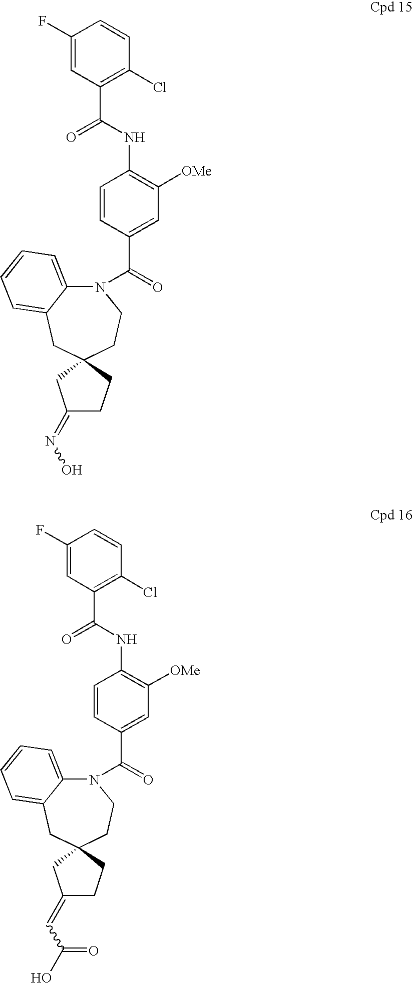

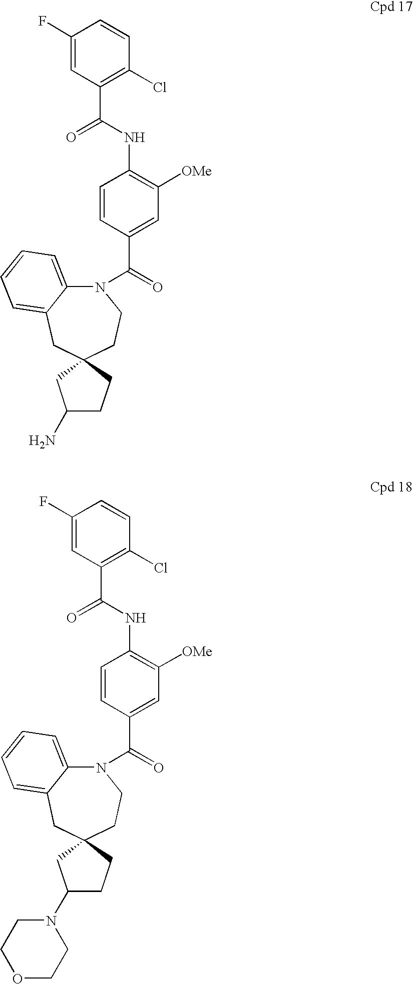

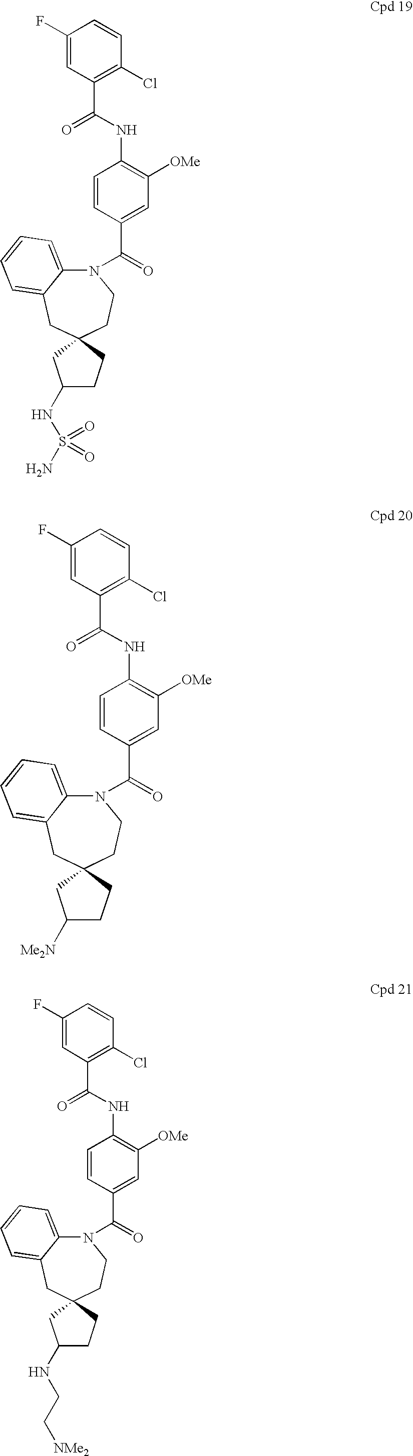

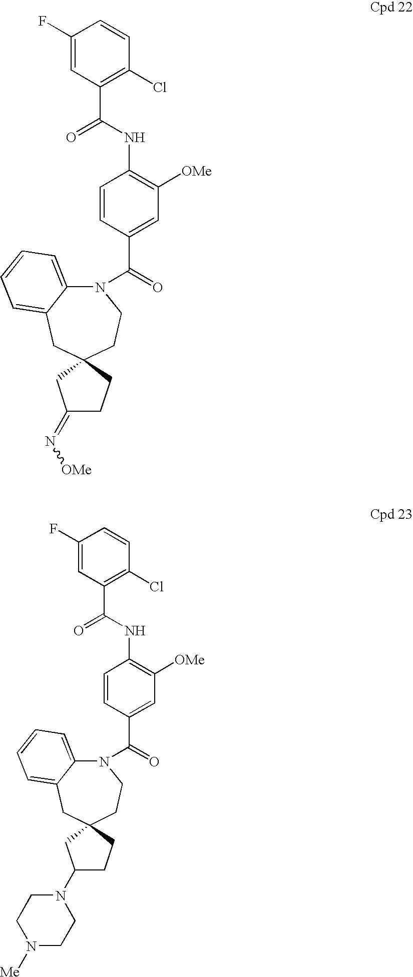

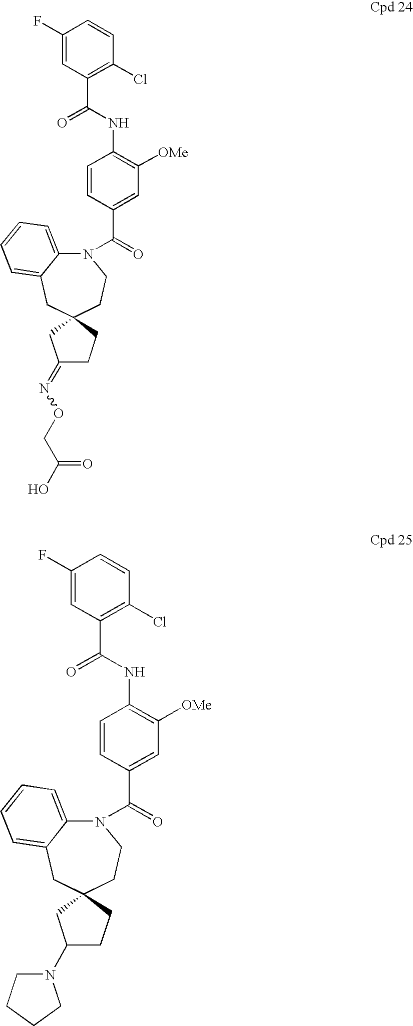

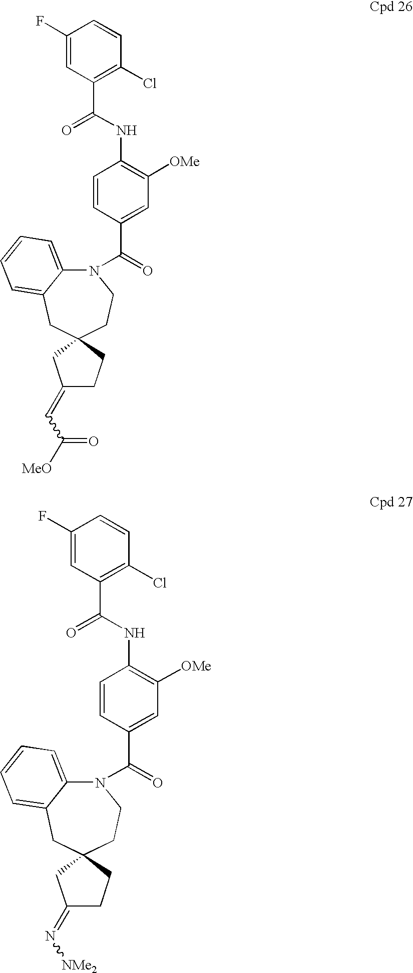

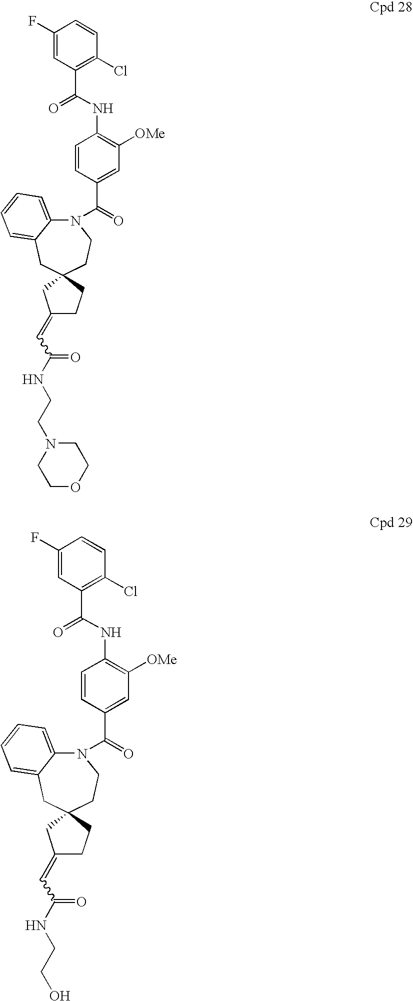

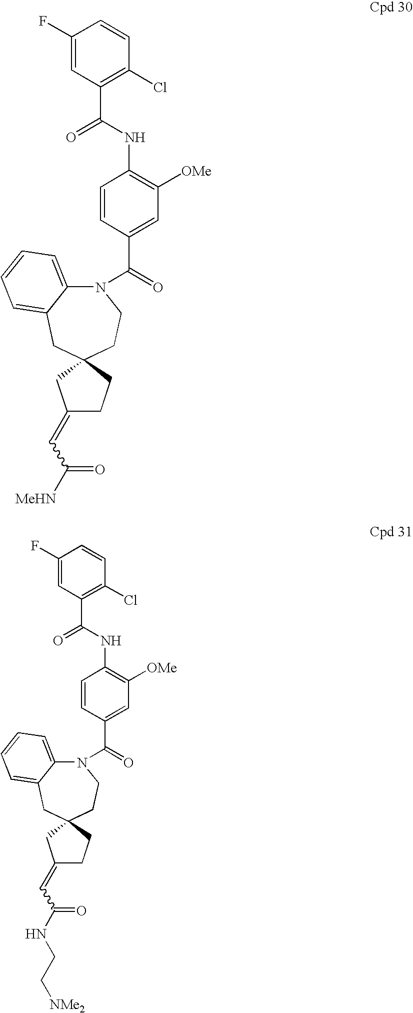

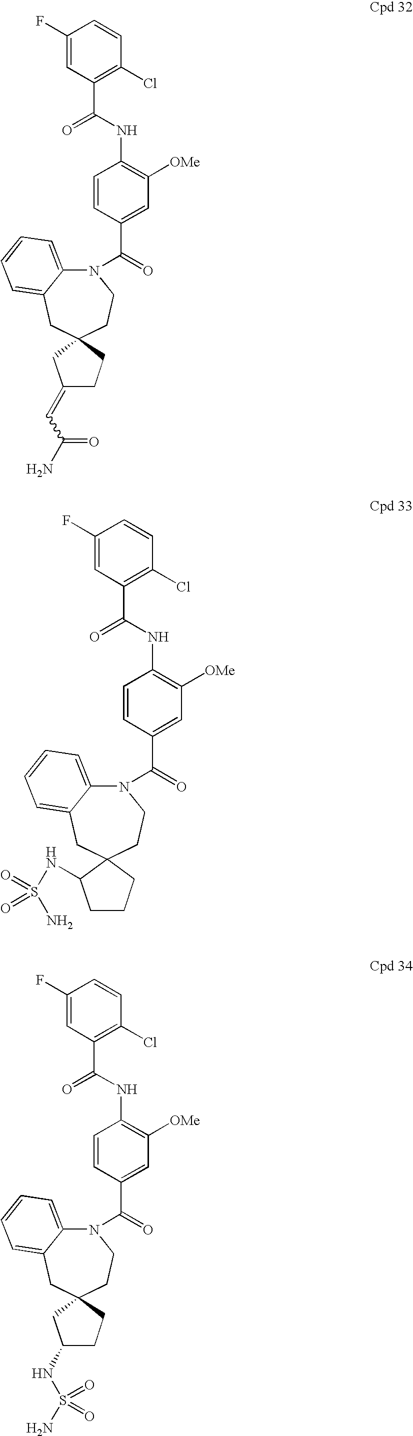

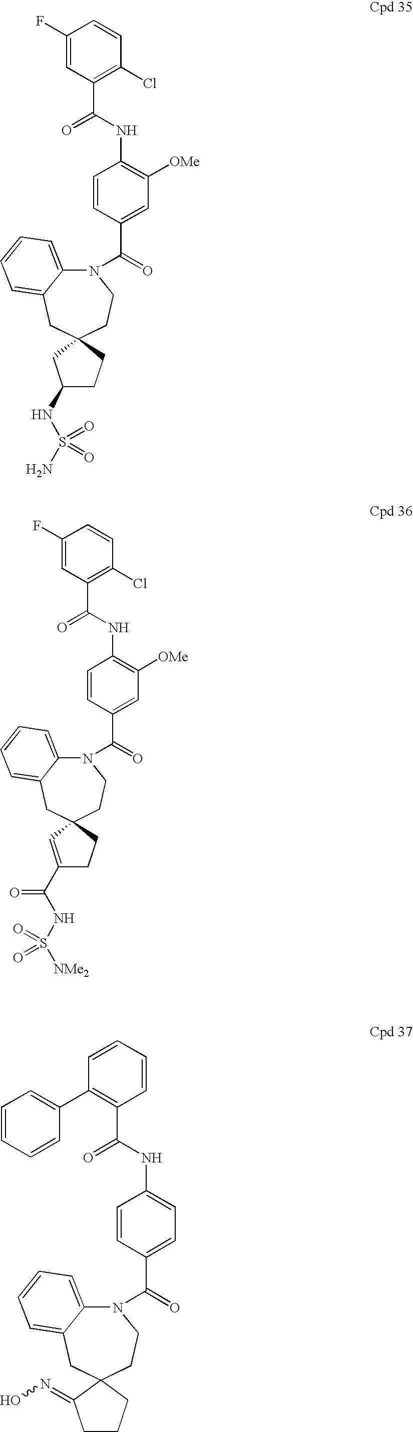

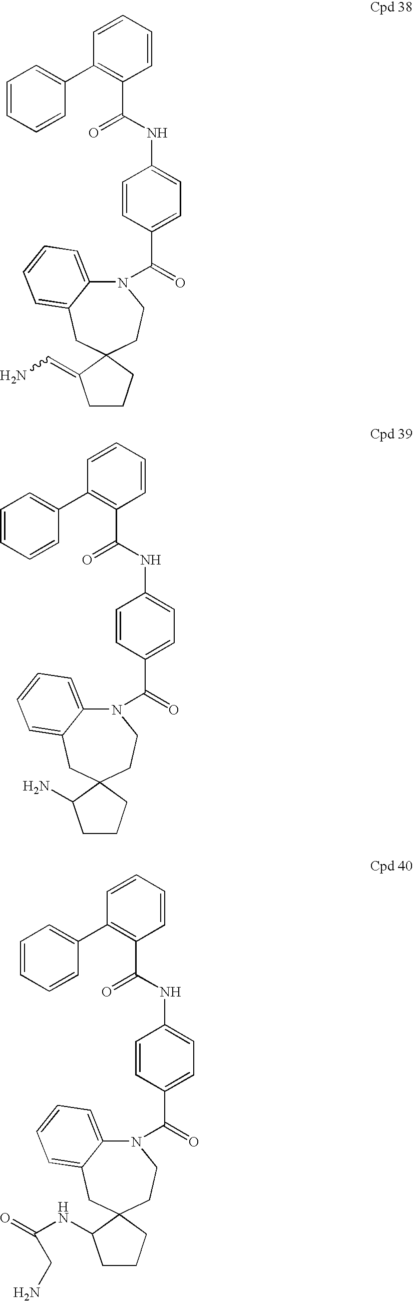

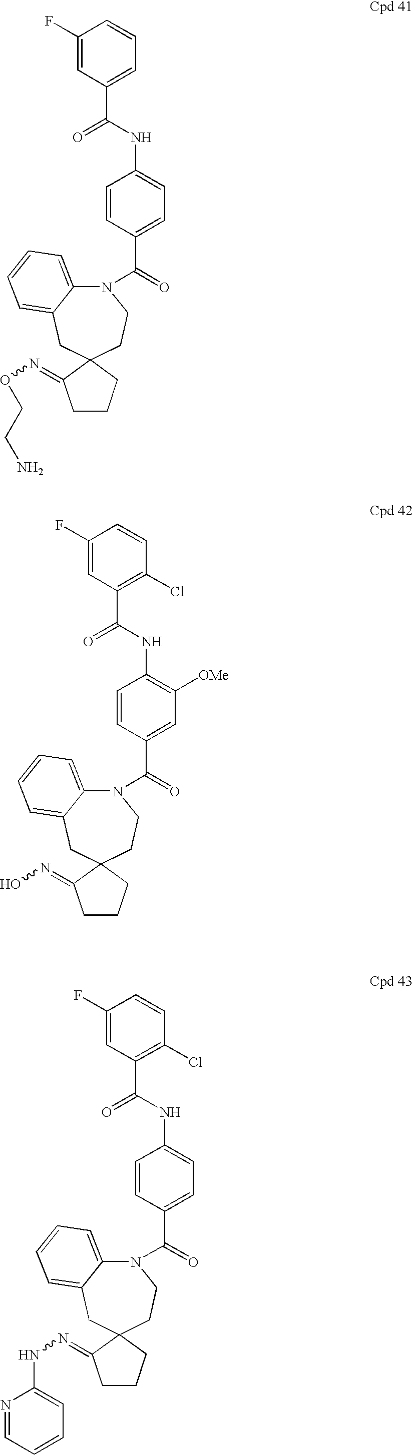

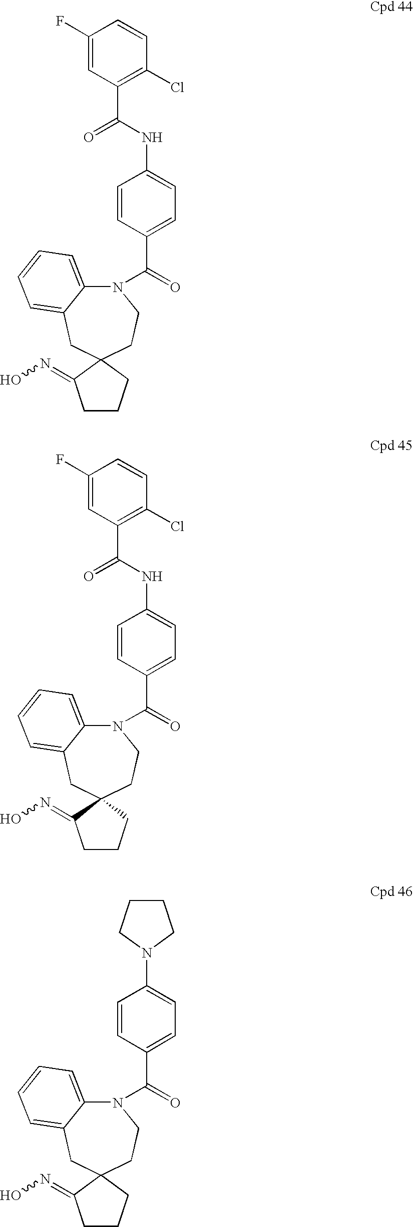

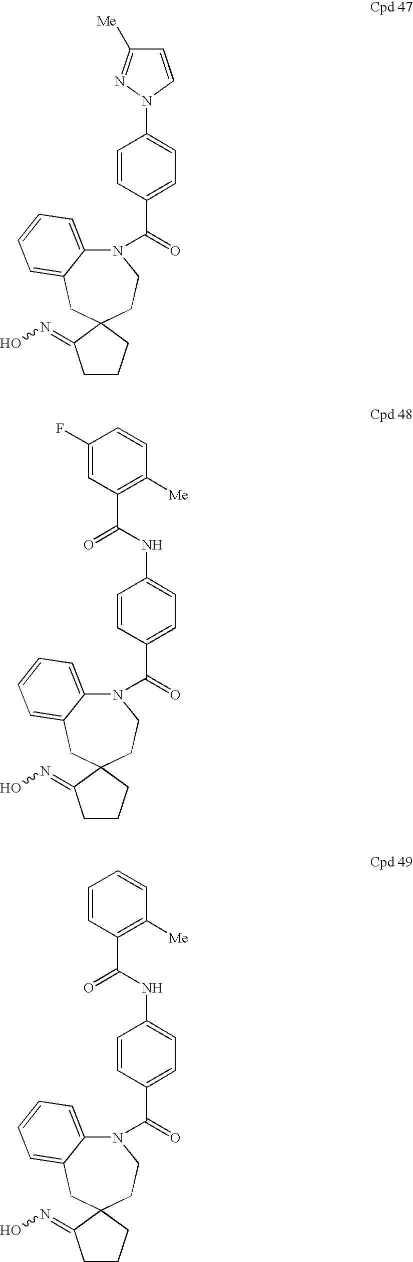

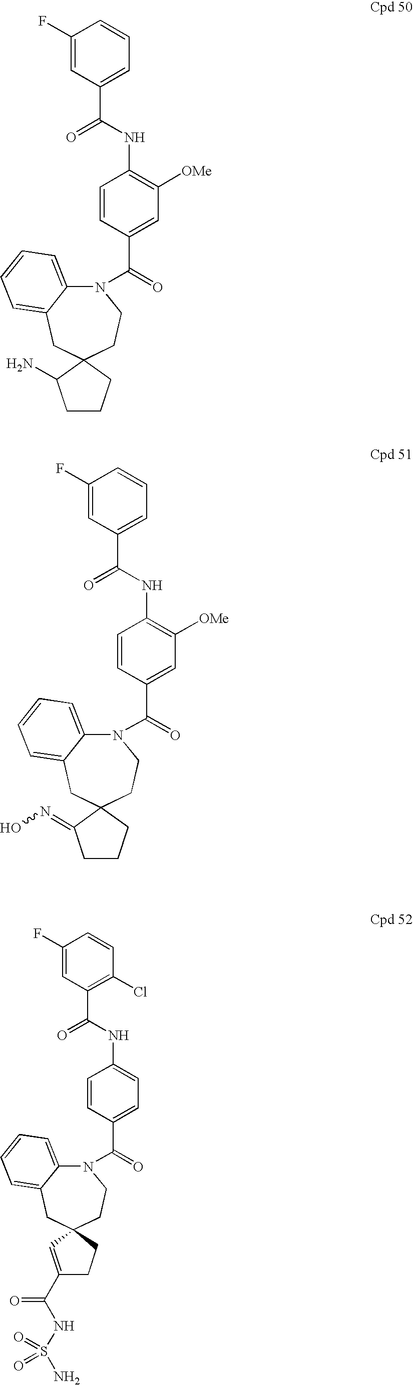

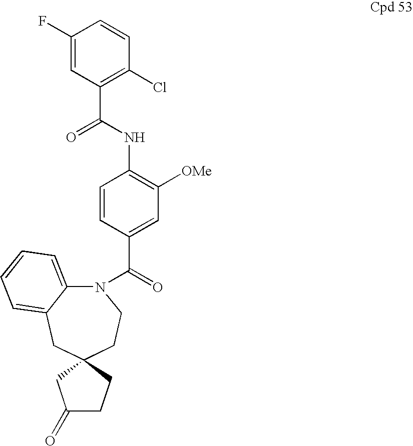

A representative compound of Formula (I) or a form thereof includes a compound selected from the group consisting of:

| Cpd | Name |
|---|---|
| 1 | 2-aminoimino-N-[4-(2-chloro-5-fluoro-phenylcarbonyl)amino- |
| | phenylcarbonyl]-spiro[cyclopentane-1,4′-benzo[b]azepane], |
| 2 | N-[4-(2-chloro-5-fluoro-phenylcarbonyl)amino-phenylcarbonyl]-2- |
| | methoxyimino-spiro[cyclopentane-1,4′-benzo[b]azepane], |
| 3 | N-[4-(2-chloro-5-fluoro-phenylcarbonyl)amino-phenylcarbonyl]-2- |
| | dimethylaminoimino-spiro[cyclopentane-1,4′-benzo[b]azepane], |
| 4 | N-[4-(2-chloro-5-fluoro-phenylcarbonyl)amino-phenylcarbonyl]-2- |
| | pyridin-3-ylcarbonylaminoimino-spiro[cyclopentane-1,4′-benzo[b]azepane], |
| 5 | N-[4-(2-chloro-5-fluoro-phenylcarbonyl)amino-phenylcarbonyl]-2-(1- |
| | methyl-1H-imidazol-2-yl)-spiro[cyclopent-2-ene-1,4′-benzo[b]azepane], |
| 6 | N-[4-(2-chloro-5-fluoro-phenylcarbonyl)amino-phenylcarbonyl]-2- |
| | dimethylamino-spiro[cyclopentane-1,4′-benzo[b]azepane], |
| 7 | (R)—N-[4-(2-chloro-5-fluoro-phenylcarbonyl)amino-phenylcarbonyl]-2- |
| | hydroxyimino-spiro[cyclopentane-1,4′-benzo[b]azepane], |
| 8 | 2-amino-N-[3-methoxy-4-(2-chloro-5-fluoro-phenylcarbonyl)amino- |
| | phenylcarbonyl]-spiro[cyclopentane-1,4′-benzo[b]azepane], |
| 9 | N-[3-methoxy-4-(2-chloro-5-fluoro-phenylcarbonyl)amino- |
| | phenylcarbonyl]-2-hydroxyimino-spiro[cyclopentane-1,4′-benzo[b]azepane], |
| 10 | N-[6-(2-chloro-5-fluoro-phenylcarbonyl)amino-pyridin-3-ylcarbonyl]-2- |
| | hydroxyimino-spiro[cyclopentane-1,4′-benzo[b]azepane], |
| 11 | (R)—N-[3-methoxy-4-(2-chloro-5-fluoro-phenylcarbonyl)amino- |
| | phenylcarbonyl]-3-tetrazol-5-yl-spiro[cyclopent-2-ene-1,4′-benzo[b]azepane], |
| 12 | (R)—N-[3-methoxy-4-(2-chloro-5-fluoro-phenylcarbonyl)amino- |
| | phenylcarbonyl]-3-(4,5-dihydro-1H-imidazol-2-yl)-spiro[cyclopent-2- |
| | ene-1,4′-benzo[b]azepane], |
| 13 | (R)-3-aminosulfonylaminocarbonyl-N-[3-methoxy-4-(2-chloro-5-fluoro- |
| | phenylcarbonyl)amino-phenylcarbonyl]-spiro[cyclopent-2-ene-1,4′- |
| | benzo[b]azepane], |
| 14 | (R)—N-[3-methoxy-4-(2-chloro-5-fluoro-phenylcarbonyl)amino- |
| | phenylcarbonyl]-3-(1H-imidazol-2-yl)-spiro[cyclopent-2-ene-1,4′- |
| | benzo[b]azepane], |
| 15 | (R)—N-[3-methoxy-4-(2-chloro-5-fluoro-phenylcarbonyl)amino- |
| | phenylcarbonyl]-3-hydroxyimino-spiro[cyclopentane-1,4′-benzo[b]azepane], |
| 16 | (R)—N-[3-methoxy-4-(2-chloro-5-fluoro-phenylcarbonyl)amino- |
| | phenylcarbonyl]-3-carboxymethylene-spiro[cyclopentane-1,4′-benzo[b]azepane], |
| 17 | (1R)-3-amino-N-[3-methoxy-4-(2-chloro-5-fluoro- |
| | phenylcarbonyl)amino-phenylcarbonyl]-spiro[cyclopentane-1,4′- |
| | benzo[b]azepane], |
| 18 | (1R)—N-[3-methoxy-4-(2-chloro-5-fluoro-phenylcarbonyl)amino- |
| | phenylcarbonyl]-3-morpholin-4-yl-spiro[cyclopentane-1,4′-benzo[b]azepane], |
| 19 | (1R)-3-aminosulfonylamino-N-[3-methoxy-4-(2-chloro-5-fluoro- |
| | phenylcarbonyl)amino-phenylcarbonyl]-spiro[cyclopentane-1,4′- |
| | benzo[b]azepane], |
| 20 | (1R)—N-[3-methoxy-4-(2-chloro-5-fluoro-phenylcarbonyl)amino- |
| | phenylcarbonyl]-3-dimethylamino-spiro[cyclopentane-1,4′- |
| | benzo[b]azepane], |
| 21 | (1R)—N-[3-methoxy-4-(2-chloro-5-fluoro-phenylcarbonyl)amino- |
| | phenylcarbonyl]-3-(2-dimethylamino-ethyl)amino-spiro[cyclopentane- |
| | 1,4′-benzo[b]azepane], |
| 22 | (R)—N-[3-methoxy-4-(2-chloro-5-fluoro-phenylcarbonyl)amino- |
| | phenylcarbonyl]-3-methoxyimino-spiro[cyclopentane-1,4′-benzo[b]azepane], |
| 23 | (1R)—N-[3-methoxy-4-(2-chloro-5-fluoro-phenylcarbonyl)amino- |
| | phenylcarbonyl]-3-(4-methyl-piperazin-1-yl)-spiro[cyclopentane-1,4′- |
| | benzo[b]azepane], |
| 24 | (R)—N-[3-methoxy-4-(2-chloro-5-fluoro-phenylcarbonyl)amino- |
| | phenylcarbonyl]-3-carboxymethoxyimino-spiro[cyclopentane-1,4′- |
| | benzo[b]azepane], |
| 25 | (1R)—N-[3-methoxy-4-(2-chloro-5-fluoro-phenylcarbonyl)amino- |
| | phenylcarbonyl]-3-(1H-pyrrolidin-1-yl)-spiro[cyclopentane-1,4′- |
| | benzo[b]azepane], |
| 26 | (R)—N-[3-methoxy-4-(2-chloro-5-fluoro-phenylcarbonyl)amino- |
| | phenylcarbonyl]-3-methoxycarbonylmethylene-spiro[cyclopentane- |
| | 1,4′-benzo[b]azepane], |
| 27 | (R)—N-[3-methoxy-4-(2-chloro-5-fluoro-phenylcarbonyl)amino- |
| | phenylcarbonyl]-3-dimethylaminoimino-spiro[cyclopentane-1,4′- |
| | benzo[b]azepane], |
| 28 | (R)—N-[3-methoxy-4-(2-chloro-5-fluoro-phenylcarbonyl)amino- |
| | phenylcarbonyl]-3-(2-morpholin-4-yl-ethyl)aminocarbonylmethylene- |
| | spiro[cyclopentane-1,4′-benzo[b]azepane], |
| 29 | (R)—N-[3-methoxy-4-(2-chloro-5-fluoro-phenylcarbonyl)amino- |
| | phenylcarbonyl]-3-(2-hydroxy-ethyl)aminocarbonylmethylene- |
| | spiro[cyclopentane-1,4′-benzo[b]azepane], |
| 30 | (R)—N-[3-methoxy-4-(2-chloro-5-fluoro-phenylcarbonyl)amino- |
| | phenylcarbonyl]-3-methylaminocarbonylmethylene- |
| | spiro[cyclopentane-1,4′-benzo[b]azepane], |
| 31 | (R)—N-[3-methoxy-4-(2-chloro-5-fluoro-phenylcarbonyl)amino- |
| | phenylcarbonyl]-3-(2-dimethylamino-ethyl)aminocarbonylmethylene- |
| | spiro[cyclopentane-1,4′-benzo[b]azepane], |
| 32 | (R)-3-aminocarbonylmethylene-N-[3-methoxy-4-(2-chloro-5-fluoro- |
| | phenylcarbonyl)amino-phenylcarbonyl]-spiro[cyclopentane-1,4′- |
| | benzo[b]azepane], |
| 33 | 2-aminosulfonylamino-N-[3-methoxy-4-(2-chloro-5-fluoro- |
| | phenylcarbonyl)amino-phenylcarbonyl]-spiro[cyclopentane-1,4′- |
| | benzo[b]azepane], |
| 34 | (1R,3S)-3-aminosulfonylamino-N-[3-methoxy-4-(2-chloro-5-fluoro- |
| | phenylcarbonyl)amino-phenylcarbonyl]-spiro[cyclopentane-1,4′- |
| | benzo[b]azepane], |
| 35 | (1R,3R)-3-aminosulfonylamino-N-[3-methoxy-4-(2-chloro-5-fluoro- |
| | phenylcarbonyl)amino-phenylcarbonyl]-spiro[cyclopentane-1,4′- |
| | benzo[b]azepane], |
| 36 | (R)—N-[3-methoxy-4-(2-chloro-5-fluoro-phenylcarbonyl)amino- |
| | phenylcarbonyl]-3-[(dimethylaminosulfonyl)aminocarbonyl]- |
| | spiro[cyclopent-2-ene-1,4′-benzo[b]azepane], |
| 37 | N-[4-(biphen-2-ylcarbonyl)amino-phenylcarbonyl]-2-hydroxyimino- |
| | spiro[cyclopentane-1,4′-benzo[b]azepane], |
| 38 | 2-aminoimino-N-[4-(biphen-2-ylcarbonyl)amino-phenylcarbonyl]- |
| | spiro[cyclopentane-1,4′-benzo[b]azepane], |
| 39 | 2-amino-N-[4-(biphen-2-ylcarbonyl)amino-phenylcarbonyl]- |
| | spiro[cyclopentane-1,4′-benzo[b]azepane], |
| 40 | 2-(aminomethylcarbonyl)amino-N-[4-(biphen-2-ylcarbonyl)amino- |
| | phenylcarbonyl]-spiro[cyclopentane-1,4′-benzo[b]azepane], |
| 41 | N-[4-(3-fluoro-phenylcarbonyl)amino-phenylcarbonyl]-2-(2-amino- |
| | ethoxy)imino-spiro[cyclopentane-1,4′-benzo[b]azepane], |
| 42 | N-[3-methoxy-4-(2-chloro-5-fluoro-phenylcarbonyl)amino- |
| | phenylcarbonyl]-2-hydroxyimino-spiro[cyclopentane-1,4′- |
| | benzo[b]azepane], |
| 43 | N-[4-(2-chloro-5-fluoro-phenylcarbonyl)amino-phenylcarbonyl]-2- |
| | pyridin-2-ylaminoimino-spiro[cyclopentane-1,4′-benzo[b]azepane], |
| 44 | N-[4-(2-chloro-5-fluoro-phenylcarbonyl)amino-phenylcarbonyl]-2- |
| | hydroxyimino-spiro[cyclopentane-1,4′-benzo[b]azepane], |
| 45 | (S)—N-[4-(2-chloro-5-fluoro-phenylcarbonyl)amino-phenylcarbonyl]-2- |
| | hydroxyimino-spiro[cyclopentane-1,4′-benzo[b]azepane], |
| 46 | N-[4-pyrrolidin-1-yl-phenylcarbonyl]-2-hydroxyimino- |
| | spiro[cyclopentane-1,4′-benzo[b]azepane], |
| 47 | N-[4-(3-methyl-1H-pyrazol-1-yl)-phenylcarbonyl]-2-hydroxyimino- |
| | spiro[cyclopentane-1,4′-benzo[b]azepane], |
| 48 | N-[4-(2-methyl-5-fluoro-phenylcarbonyl)amino-phenylcarbonyl]-2- |
| | hydroxyimino-spiro[cyclopentane-1,4′-benzo[b]azepane], |
| 49 | N-[4-(2-methyl-phenylcarbonyl)amino-phenylcarbonyl]-2- |
| | hydroxyimino-spiro[cyclopentane-1,4′-benzo[b]azepane], |
| 50 | 2-amino-N-[3-methoxy-4-(3-fluoro-phenylcarbonyl)amino- |
| | phenylcarbonyl]-spiro[cyclopentane-1,4′-benzo[b]azepane], |
| 51 | N-[3-methoxy-4-(3-fluoro-phenylcarbonyl)amino-phenylcarbonyl]-2- |
| | hydroxyimino-spiro[cyclopentane-1,4′-benzo[b]azepane], |
| 52 | (R)-3-aminosulfonylaminocarbonyl-N-[4-(2-chloro-5-fluoro- |
| | phenylcarbonyl)amino-phenylcarbonyl]-spiro[cyclopent-2-ene-1,4′- |
| | benzo[b]azepane], and |
| 53 | (R)—N-[3-methoxy-4-(2-chloro-5-fluoro-phenylcarbonyl)amino- |
| | phenylcarbonyl]-3-oxo-spiro[cyclopentane-1,4′-benzo[b]azepane]. |

A representative compound of Formula (I) or a form thereof includes a compound selected from the group consisting of:

| Cpd | Name |
|---|---|
| 1 | 2-aminoimino-N-[4-(2-chloro-5-fluoro-phenylcarbonyl)amino- |
| | phenylcarbonyl]-spiro[cyclopentane-1,4′-benzo[b]azepane], |
| 2 | N-[4-(2-chloro-5-fluoro-phenylcarbonyl)amino-phenylcarbonyl]-2- |
| | methoxyimino-spiro[cyclopentane-1,4′-benzo[b]azepane], |
| 3 | N-[4-(2-chloro-5-fluoro-phenylcarbonyl)amino-phenylcarbonyl]-2- |
| | dimethylaminoimino-spiro[cyclopentane-1,4′-benzo[b]azepane], |
| 4 | N-[4-(2-chloro-5-fluoro-phenylcarbonyl)amino-phenylcarbonyl]-2- |
| | pyridin-3-ylcarbonylaminoimino-spiro[cyclopentane-1,4′-benzo[b]azepane], |
| 5 | N-[4-(2-chloro-5-fluoro-phenylcarbonyl)amino-phenylcarbonyl]-2-(1- |
| | methyl-1H-imidazol-2-yl)-spiro[cyclopent-2-ene-1,4′-benzo[b]azepane], |
| 6 | N-[4-(2-chloro-5-fluoro-phenylcarbonyl)amino-phenylcarbonyl]-2- |
| | dimethylamino-spiro[cyclopentane-1,4′-benzo[b]azepane], |
| 7 | (R)—N-[4-(2-chloro-5-fluoro-phenylcarbonyl)amino-phenylcarbonyl]-2- |
| | hydroxyimino-spiro[cyclopentane-1,4′-benzo[b]azepane], |
| 8 | 2-amino-N-[3-methoxy-4-(2-chloro-5-fluoro-phenylcarbonyl)amino- |
| | phenylcarbonyl]-spiro[cyclopentane-1,4′-benzo[b]azepane], |
| 9 | N-[3-methoxy-4-(2-chloro-5-fluoro-phenylcarbonyl)amino- |
| | phenylcarbonyl]-2-hydroxyimino-spiro[cyclopentane-1,4′-benzo[b]azepane], |
| 10 | N-[6-(2-chloro-5-fluoro-phenylcarbonyl)amino-pyridin-3-ylcarbonyl]-2- |
| | hydroxyimino-spiro[cyclopentane-1,4′-benzo[b]azepane], |
| 11 | (R)—N-[3-methoxy-4-(2-chloro-5-fluoro-phenylcarbonyl)amino- |
| | phenylcarbonyl]-3-tetrazol-5-yl-spiro[cyclopent-2-ene-1,4′-benzo[b]azepane], |
| 12 | (R)—N-[3-methoxy-4-(2-chloro-5-fluoro-phenylcarbonyl)amino- |
| | phenylcarbonyl]-3-(4,5-dihydro-1H-imidazol-2-yl)-spiro[cyclopent-2- |
| | ene-1,4′-benzo[b]azepane], |
| 13 | (R)-3-aminosulfonylaminocarbonyl-N-[3-methoxy-4-(2-chloro-5-fluoro- |
| | phenylcarbonyl)amino-phenylcarbonyl]-spiro[cyclopent-2-ene-1,4′-benzo[b]azepane], |
| 14 | (R)—N-[3-methoxy-4-(2-chloro-5-fluoro-phenylcarbonyl)amino- |
| | phenylcarbonyl]-3-(1H-imidazol-2-yl)-spiro[cyclopent-2-ene-1,4′-benzo[b]azepane], |
| 15 | (R)—N-[3-methoxy-4-(2-chloro-5-fluoro-phenylcarbonyl)amino- |
| | phenylcarbonyl]-3-hydroxyimino-spiro[cyclopentane-1,4′-benzo[b]azepane], |
| 16 | (R)—N-[3-methoxy-4-(2-chloro-5-fluoro-phenylcarbonyl)amino- |
| | phenylcarbonyl]-3-carboxymethylene-spiro[cyclopentane-1,4′-benzo[b]azepane], |
| 18 | (1R)—N-[3-methoxy-4-(2-chloro-5-fluoro-phenylcarbonyl)amino- |
| | phenylcarbonyl]-3-morpholin-4-yl-spiro[cyclopentane-1,4′-benzo[b]azepane], |
| 19 | (1R)-3-aminosulfonylamino-N-[3-methoxy-4-(2-chloro-5-fluoro- |
| | phenylcarbonyl)amino-phenylcarbonyl]-spiro[cyclopentane-1,4′-benzo[b]azepane], |
| 20 | (1R)—N-[3-methoxy-4-(2-chloro-5-fluoro-phenylcarbonyl)amino- |
| | phenylcarbonyl]-3-dimethylamino-spiro[cyclopentane-1,4′- |
| | benzo[b]azepane], |
| 21 | (1R)—N-[3-methoxy-4-(2-chloro-5-fluoro-phenylcarbonyl)amino- |
| | phenylcarbonyl]-3-(2-dimethylamino-ethyl)amino-spiro[cyclopentane- |
| | 1,4′-benzo[b]azepane], |
| 22 | (R)—N-[3-methoxy-4-(2-chloro-5-fluoro-phenylcarbonyl)amino- |
| | phenylcarbonyl]-3-methoxyimino-spiro[cyclopentane-1,4′- |
| | benzo[b]azepane], |
| 24 | (R)—N-[3-methoxy-4-(2-chloro-5-fluoro-phenylcarbonyl)amino- |
| | phenylcarbonyl]-3-carboxymethoxyimino-spiro[cyclopentane-1,4′- |
| | benzo[b]azepane], |
| 26 | (R)—N-[3-methoxy-4-(2-chloro-5-fluoro-phenylcarbonyl)amino- |
| | phenylcarbonyl]-3-methoxycarbonylmethylene-spiro[cyclopentane- |
| | 1,4′-benzo[b]azepane], |
| 27 | (R)—N-[3-methoxy-4-(2-chloro-5-fluoro-phenylcarbonyl)amino- |
| | phenylcarbonyl]-3-dimethylaminoimino-spiro[cyclopentane-1,4′- |
| | benzo[b]azepane], |
| 28 | (R)—N-[3-methoxy-4-(2-chloro-5-fluoro-phenylcarbonyl)amino- |
| | phenylcarbonyl]-3-(2-morpholin-4-yl-ethyl)aminocarbonylmethylene- |
| | spiro[cyclopentane-1,4′-benzo[b]azepane], |
| 29 | (R)—N-[3-methoxy-4-(2-chloro-5-fluoro-phenylcarbonyl)amino- |
| | phenylcarbonyl]-3-(2-hydroxy-ethyl)aminocarbonylmethylene- |
| | spiro[cyclopentane-1,4′-benzo[b]azepane], |
| 30 | (R)—N-[3-methoxy-4-(2-chloro-5-fluoro-phenylcarbonyl)amino- |
| | phenylcarbonyl]-3-methylaminocarbonylmethylene- |
| | spiro[cyclopentane-1,4′-benzo[b]azepane], |
| 31 | (R)—N-[3-methoxy-4-(2-chloro-5-fluoro-phenylcarbonyl)amino- |
| | phenylcarbonyl]-3-(2-dimethylamino-ethyl)aminocarbonylmethylene- |
| | spiro[cyclopentane-1,4′-benzo[b]azepane], |
| 32 | (R)-3-aminocarbonylmethylene-N-[3-methoxy-4-(2-chloro-5-fluoro- |
| | phenylcarbonyl)amino-phenylcarbonyl]-spiro[cyclopentane-1,4′- |
| | benzo[b]azepane], |
| 33 | 2-aminosulfonylamino-N-[3-methoxy-4-(2-chloro-5-fluoro- |
| | phenylcarbonyl)amino-phenylcarbonyl]-spiro[cyclopentane-1,4′- |
| | benzo[b]azepane], |
| 34 | (1R,3S)-3-aminosulfonylamino-N-[3-methoxy-4-(2-chloro-5-fluoro- |
| | phenylcarbonyl)amino-phenylcarbonyl]-spiro[cyclopentane-1,4′- |
| | benzo[b]azepane], |
| 35 | (1R,3R)-3-aminosulfonylamino-N-[3-methoxy-4-(2-chloro-5-fluoro- |
| | phenylcarbonyl)amino-phenylcarbonyl]-spiro[cyclopentane-1,4′- |
| | benzo[b]azepane], |
| 36 | (R)—N-[3-methoxy-4-(2-chloro-5-fluoro-phenylcarbonyl)amino- |
| | phenylcarbonyl]-3-[(dimethylaminosulfonyl)aminocarbonyl]- |
| | spiro[cyclopent-2-ene-1,4′-benzo[b]azepane], |
| 37 | N-[4-(biphen-2-ylcarbonyl)amino-phenylcarbonyl]-2-hydroxyimino- |
| | spiro[cyclopentane-1,4′-benzo[b]azepane], |
| 38 | 2-aminoimino-N-[4-(biphen-2-ylcarbonyl)amino-phenylcarbonyl]- |
| | spiro[cyclopentane-1,4′-benzo[b]azepane], |
| 39 | 2-amino-N-[4-(biphen-2-ylcarbonyl)amino-phenylcarbonyl]- |
| | spiro[cyclopentane-1,4′-benzo[b]azepane], |
| 40 | 2-(aminomethylcarbonyl)amino-N-[4-(biphen-2-ylcarbonyl)amino- |
| | phenylcarbonyl]-spiro[cyclopentane-1,4′-benzo[b]azepane], |
| 41 | N-[4-(3-fluoro-phenylcarbonyl)amino-phenylcarbonyl]-2-(2-amino- |
| | ethoxy)imino-spiro[cyclopentane-1,4′-benzo[b]azepane], |
| 42 | N-[3-methoxy-4-(2-chloro-5-fluoro-phenylcarbonyl)amino- |
| | phenylcarbonyl]-2-hydroxyimino-spiro[cyclopentane-1,4′- |
| | benzo[b]azepane], |
| 43 | N-[4-(2-chloro-5-fluoro-phenylcarbonyl)amino-phenylcarbonyl]-2- |
| | pyridin-2-ylaminoimino-spiro[cyclopentane-1,4′-benzo[b]azepane], |
| 44 | N-[4-(2-chloro-5-fluoro-phenylcarbonyl)amino-phenylcarbonyl]-2- |
| | hydroxyimino-spiro[cyclopentane-1,4′-benzo[b]azepane], |
| 45 | (S)—N-[4-(2-chloro-5-fluoro-phenylcarbonyl)amino-phenylcarbonyl]-2- |
| | hydroxyimino-spiro[cyclopentane-1,4′-benzo[b]azepane], |
| 46 | N-[4-pyrrolidin-1-yl-phenylcarbonyl]-2-hydroxyimino- |
| | spiro[cyclopentane-1,4′-benzo[b]azepane], |
| 47 | N-[4-(3-methyl-1H-pyrazol-1-yl)-phenylcarbonyl]-2-hydroxyimino- |
| | spiro[cyclopentane-1,4′-benzo[b]azepane], |
| 48 | N-[4-(2-methyl-5-fluoro-phenylcarbonyl)amino-phenylcarbonyl]-2- |
| | hydroxyimino-spiro[cyclopentane-1,4′-benzo[b]azepane], |
| 49 | N-[4-(2-methyl-phenylcarbonyl)amino-phenylcarbonyl]-2- |
| | hydroxyimino-spiro[cyclopentane-1,4′-benzo[b]azepane], |
| 50 | 2-amino-N-[3-methoxy-4-(3-fluoro-phenylcarbonyl)amino- |
| | phenylcarbonyl]-spiro[cyclopentane-1,4′-benzo[b]azepane], |
| 51 | N-[3-methoxy-4-(3-fluoro-phenylcarbonyl)amino-phenylcarbonyl]-2- |
| | hydroxyimino-spiro[cyclopentane-1,4′-benzo[b]azepane], and |
| 52 | (R)-3-aminosulfonylaminocarbonyl-N-[4-(2-chloro-5-fluoro- |
| | phenylcarbonyl)amino-phenylcarbonyl]-spiro[cyclopent-2-ene-1,4′- |
| | benzo[b]azepane]. |

A representative compound of Formula (I) or a form thereof includes a compound selected from the group consisting of:

| Cpd | Name |
|---|---|
| 1 | 2-aminoimino-N-[4-(2-chloro-5-fluoro-phenylcarbonyl)amino-phenylcarbonyl]- |
| | spiro[cyclopentane-1,4′-benzo[b]azepane], |
| 2 | N-[4-(2-chloro-5-fluoro-phenylcarbonyl)amino-phenylcarbonyl]-2- |
| | methoxyimino-spiro[cyclopentane-1,4′-benzo[b]azepane], |
| 3 | N-[4-(2-chloro-5-fluoro-phenylcarbonyl)amino-phenylcarbonyl]-2- |
| | dimethylaminoimino-spiro[cyclopentane-1,4′-benzo[b]azepane], |
| 4 | N-[4-(2-chloro-5-fluoro-phenylcarbonyl)amino-phenylcarbonyl]-2- |
| | ylcarbonylaminoimino-spiro[cyclopentane-1,4′-pyridin-3- |
| | benzo[b]azepane], |
| 5 | N-[4-(2-chloro-5-fluoro-phenylcarbonyl)amino-phenylcarbonyl]-2-(1- |
| | methyl-1H-imidazol-2-yl)-spiro[cyclopent-2-ene-1,4′-benzo[b]azepane], |
| 6 | N-[4-(2-chloro-5-fluoro-phenylcarbonyl)amino-phenylcarbonyl]-2- |
| | dimethylamino-spiro[cyclopentane-1,4′-benzo[b]azepane], |
| 7 | (R)—N-[4-(2-chloro-5-fluoro-phenylcarbonyl)amino-phenylcarbonyl]-2-hydroxyimino- |
| | spiro[cyclopentane-1,4′-benzo[b]azepane], |
| 8 | 2-amino-N-[3-methoxy-4-(2-chloro-5-fluoro-phenylcarbonyl)amino- |
| | phenylcarbonyl]-spiro[cyclopentane-1,4′-benzo[b]azepane], |
| 9 | N-[3-methoxy-4-(2-chloro-5-fluoro-phenylcarbonyl)amino- |
| | phenylcarbonyl]-2-hydroxyimino-spiro[cyclopentane-1,4′- |
| | benzo[b]azepane], |
| 10 | N-[6-(2-chloro-5-fluoro-phenylcarbonyl)amino-pyridin-3-ylcarbonyl]-2-hydroxyimino- |
| | spiro[cyclopentane-1,4′-benzo[b]azepane], |
| 11 | (R)—N-[3-methoxy-4-(2-chloro-5-fluoro-phenylcarbonyl)amino- |
| | phenylcarbonyl]-3-tetrazol-5-yl-spiro[cyclopent-2-ene-1,4′- |
| | benzo[b]azepane], |
| 13 | (R)-3-aminosulfonylaminocarbonyl-N-[3-methoxy-4-(2-chloro-5-fluoro- |
| | phenylcarbonyl)amino-phenylcarbonyl]-spiro[cyclopent-2-ene-1,4′- |
| | benzo[b]azepane], |
| 14 | (R)—N-[3-methoxy-4-(2-chloro-5-fluoro-phenylcarbonyl)amino- |
| | phenylcarbonyl]-3-(1H-imidazol-2-yl)-spiro[cyclopent-2-ene-1,4′- |
| | benzo[b]azepane], |
| 15 | (R)—N-[3-methoxy-4-(2-chloro-5-fluoro-phenylcarbonyl)amino- |
| | phenylcarbonyl]-3-hydroxyimino-spiro[cyclopentane-1,4′- |
| | benzo[b]azepane], |
| 16 | (R)—N-[3-methoxy-4-(2-chloro-5-fluoro-phenylcarbonyl)amino- |
| | phenylcarbonyl]-3-carboxymethylene-spiro[cyclopentane-1,4′- |
| | benzo[b]azepane], |
| 26 | (R)—N-[3-methoxy-4-(2-chloro-5-fluoro-phenylcarbonyl)amino- |
| | phenylcarbonyl]-3-methoxycarbonylmethylene-spiro[cyclopentane- |
| | 1,4′-benzo[b]azepane], |
| 29 | (R)—N-[3-methoxy-4-(2-chloro-5-fluoro-phenylcarbonyl)amino- |
| | phenylcarbonyl]-3-(2-hydroxy-ethyl)aminocarbonylmethylene- |
| | spiro[cyclopentane-1,4′-benzo[b]azepane], |
| 30 | (R)—N-[3-methoxy-4-(2-chloro-5-fluoro-phenylcarbonyl)amino- |
| | phenylcarbonyl]-3-methylaminocarbonylmethylene- |
| | spiro[cyclopentane-1,4′-benzo[b]azepane], |
| 32 | (R)-3-aminocarbonylmethylene-N-[3-methoxy-4-(2-chloro-5-fluoro- |
| | phenylcarbonyl)amino-phenylcarbonyl]-spiro[cyclopentane-1,4′- |
| | benzo[b]azepane], |
| 33 | 2-aminosulfonylamino-N-[3-methoxy-4-(2-chloro-5-fluoro- |
| | phenylcarbonyl)amino-phenylcarbonyl]-spiro[cyclopentane-1,4′- |
| | benzo[b]azepane], |
| 35 | (1R,3R)-3-aminosulfonylamino-N-[3-methoxy-4-(2-chloro-5-fluoro- |
| | phenylcarbonyl)amino-phenylcarbonyl]-spiro[cyclopentane-1,4′- |
| | benzo[b]azepane], |
| 37 | N-[4-(biphen-2-ylcarbonyl)amino-phenylcarbonyl]-2-hydroxyimino- |
| | spiro[cyclopentane-1,4′-benzo[b]azepane], |
| 38 | 2-aminoimino-N-[4-(biphen-2-ylcarbonyl)amino-phenylcarbonyl]- |
| | spiro[cyclopentane-1,4′-benzo[b]azepane], |
| 39 | 2-amino-N-[4-(biphen-2-ylcarbonyl)amino-phenylcarbonyl]- |
| | spiro[cyclopentane-1,4′-benzo[b]azepane], |
| 40 | 2-(aminomethylcarbonyl)amino-N-[4-(biphen-2-ylcarbonyl)amino- |
| | phenylcarbonyl]-spiro[cyclopentane-1,4′-benzo[b]azepane], |
| 41 | N-[4-(3-fluoro-phenylcarbonyl)amino-phenylcarbonyl]-2-(2-amino- |
| | ethoxy)imino-spiro[cyclopentane-1,4′-benzo[b]azepane], |
| 42 | N-[3-methoxy-4-(2-chloro-5-fluoro-phenylcarbonyl)amino- |
| | phenylcarbonyl]-2-hydroxyimino-spiro[cyclopentane-1,4′- |
| | benzo[b]azepane], |
| 43 | N-[4-(2-chloro-5-fluoro-phenylcarbonyl)amino-phenylcarbonyl]-2- |
| | pyridin-2-ylaminoimino-spiro[cyclopentane-1,4′-benzo[b]azepane], |
| 44 | N-[4-(2-chloro-5-fluoro-phenylcarbonyl)amino-phenylcarbonyl]-2- |
| | hydroxyimino-spiro[cyclopentane-1,4′-benzo[b]azepane], |
| 48 | N-[4-(2-methyl-5-fluoro-phenylcarbonyl)amino-phenylcarbonyl]-2- |
| | hydroxyimino-spiro[cyclopentane-1,4′-benzo[b]azepane], |
| 49 | N-[4-(2-methyl-phenylcarbonyl)amino-phenylcarbonyl]-2- |
| | hydroxyimino-spiro[cyclopentane-1,4′-benzo[b]azepane], |
| 51 | N-[3-methoxy-4-(3-fluoro-phenylcarbonyl)amino-phenylcarbonyl]-2- |
| | hydroxyimino-spiro[cyclopentane-1,4′-benzo[b]azepane], and |
| 52 | (R)-3-aminosulfonylaminocarbonyl-N-[4-(2-chloro-5-fluoro- |
| | phenylcarbonyl)amino-phenylcarbonyl]-spiro[cyclopent-2-ene-1,4′- |
| | benzo[b]azepane]. |

A representative compound of Formula (I) or a form thereof includes a compound selected from the group consisting of:

| Cpd | Name |
|---|---|
| 1 | 2-aminoimino-N-[4-(2-chloro-5-fluoro-phenylcarbonyl)amino- |
| | phenylcarbonyl]-spiro[cyclopentane-1,4′-benzo[b]azepane], |
| 2 | N-[4-(2-chloro-5-fluoro-phenylcarbonyl)amino-phenylcarbonyl]-2-methoxyimino- |
| | spiro[cyclopentane-1,4′-benzo[b]azepane], |
| 3 | N-[4-(2-chloro-5-fluoro-phenylcarbonyl)amino-phenylcarbonyl]-2- |
| | dimethylaminoimino-spiro[cyclopentane-1,4′-benzo[b]azepane], |
| 5 | N-[4-(2-chloro-5-fluoro-phenylcarbonyl)amino-phenylcarbonyl]-2-(1-methyl-1H- |
| | imidazol-2-yl)-spiro[cyclopent-2-ene-1,4′-benzo[b]azepane], |
| 7 | (R)—N-[4-(2-chloro-5-fluoro-phenylcarbonyl)amino-phenylcarbonyl]-2- |
| | hydroxyimino-spiro[cyclopentane-1,4′-benzo[b]azepane], |
| 41 | N-[4-(3-fluoro-phenylcarbonyl)amino-phenylcarbonyl]-2-(2-amino-ethoxy)imino- |
| | spiro[cyclopentane-1,4′-benzo[b]azepane], |
| 43 | N-[4-(2-chloro-5-fluoro-phenylcarbonyl)amino-phenylcarbonyl]-2- |
| | pyridin-2-ylaminoimino-spiro[cyclopentane-1,4′-benzo[b]azepane], and |
| 44 | N-[4-(2-chloro-5-fluoro-phenylcarbonyl)amino-phenylcarbonyl]-2-hydroxyimino- |
| | spiro[cyclopentane-1,4′-benzo[b]azepane]. |

### Chemical Definitions & Nomenclature

It should also be noted that any atom with unsatisfied valences in the text, schemes, examples, structural formulae and any tables herein is assumed to have the hydrogen atom or atoms to satisfy the valences.

As used herein, the following terms are intended to have the following definitions. The definitions herein may specify that a chemical term has an indicated formula. The particular formula provided is not intended to limit the scope of the invention, but is provided as an illustration of the term. The scope of the per se definition of the term is intended to include the plurality of variations expected to be included by one of ordinary skill in the art.

The term “C₁₋₈alkyl” means a saturated aliphatic branched or straight-chain hydrocarbon radical or linking group having from 1 up to 8 carbon atoms in a linear or branched arrangement, wherein the radical is derived by the removal of one hydrogen atom from a carbon atom and the linking group is derived by the removal of one hydrogen atom from each of two carbon atoms in the chain. The term “C₁₋₈alkyl” also includes a “C₁₋₆alkyl” and “C₁₋₄alkyl” radical or linking group having from 1 up to 6 carbon atoms and 1 up to 4 carbon atoms, respectively, such as methyl, ethyl, 1-propyl, 2-propyl, 1-butyl, 2-butyl, tert-butyl, 1-pentyl, 2-pentyl, 3-pentyl, 1-hexyl, 2-hexyl, 3-hexyl, 1-heptyl, 2-heptyl, 3-heptyl, 1-octyl, 2-octyl, 3-octyl and the like. Alkyl radicals may be attached to a core molecule by any atom where allowed by available valences.

The term “C₁₋₈alkoxy” means an alkyl radical or linking group having from 1 up to 8 carbon atoms in a linear or branched arrangement, wherein the radical or linking group is attached through an oxygen linking atom, as in the formula: —O—C₁₋₈alkyl. The term “C₁₋₈alkoxy” also includes a “C₁₋₆alkoxy” and “C₁₋₄alkoxy” radical or linking group having from 1 up to 6 carbon atoms and from 1 up to 4 carbon atoms respectively, such as methoxy, ethoxy, propoxy, butoxy and the like. An alkoxy radical may be attached to a core molecule by any atom where allowed by available valences.

The term “aryl” means an unsaturated, aromatic hydrocarbon ring system radical. Examples of aryl ring systems include phenyl, naphthalenyl, azulenyl, anthracenyl and the like. An aryl radical may be attached to a core molecule by any atom where allowed by available valences.

The term “hetero”, when used as a prefix for a ring system, refers to the replacement of at least one carbon atom member in the ring system with a heteroatom selected from N, O, S, S(O), or SO₂. A hetero ring may have 1, 2, 3 or 4 carbon atom members replaced by a nitrogen atom. Alternatively, a ring may have 1, 2 or 3 nitrogen atom members and 1 oxygen or sulfur atom member. Alternatively, a ring may have 1 oxygen or sulfur atom member. Alternatively, up to two adjacent ring members may be heteroatoms, wherein one heteroatom is nitrogen and the other heteroatom is selected from N, S or O.

The term “heterocyclyl” means a saturated or partially unsaturated “hetero” ring system radical. Heterocyclyl ring systems include 2H-pyrrole, 2-pyrrolinyl, 3-pyrrolinyl, pyrrolidinyl, 1,3-dioxolanyl, 2-imidazolinyl (also referred to as 4,5-dihydro-1H-imidazolyl), imidazolidinyl, 2-pyrazolinyl, pyrazolidinyl, tetrazolyl, tetrazolidinyl, piperidinyl, 1,4-dioxanyl, morpholinyl, 1,4-dithianyl, thiomorpholinyl, piperazinyl, azetidinyl, azepanyl, hexahydro-1,4-diazepinyl, hexahydro-1,4-oxazepanyl, tetrahydro-furanyl, tetrahydro-thienyl, tetrahydro-pyranyl, tetrahydro-pyridazinyl and the like. The term “heterocyclyl” also includes a benzofused-heterocyclyl ring system radical and the like, such as indolinyl (also referred to as 2,3-dihydro-indolyl), benzo[1,3]dioxolyl, 2,3-dihydro-1,4-benzodioxinyl, 2,3-dihydro-benzofuranyl, 1,2-dihydro-phthalazinyl and the like. A heterocyclyl radical may be attached to a core molecule by any atom where allowed by available valences.

The term “benzofused-heterocyclyl” means a heterocyclyl ring system radical having a benzene ring fused on the ring system on adjacent carbons. A benzofused-heterocyclyl radical may be attached to a core molecule by any atom where allowed by available valences.

The term “heteroaryl” means a monovalent, unsaturated aromatic “hetero” ring system radical. Heteroaryl ring systems include furyl, thienyl, pyrrolyl, oxazolyl, thiazolyl, imidazolyl, pyrazolyl, isoxazolyl, isothiazolyl, oxadiazolyl, triazolyl, thiadiazolyl, pyridinyl, pyridazinyl, pyrimidinyl, pyrazinyl and the like. The term “heteroaryl” also includes a benzofused-heteroaryl ring system radical and the like, such as indolizinyl, indolyl, azaindolyl, isoindolyl, benzofuranyl, benzothienyl, indazolyl, azaindazolyl, benzoimidazolyl, benzothiazolyl, benzoxazolyl, benzoisoxazolyl, benzothiadiazolyl, benzotriazolyl, purinyl, 4H-quinolizinyl, quinolinyl, isoquinolinyl, cinnolinyl, phthalazinyl, quinazolinyl, quinoxalinyl, 1,8-naphthyridinyl, pteridinyl and the like. A heteroaryl radical may be attached to a core molecule by any atom where allowed by available valences.

The term “benzofused-heteroaryl” means a heteroaryl ring system radical having a benzene ring fused on the ring system on adjacent carbons. Examples of benzofused-heteroaryl in compounds representative of the present invention include indolyl and quinolinyl. A benzofused-heteroaryl radical may be attached to a core molecule by any atom where when allowed by available valences.

The term “C₁₋₄alkoxy-carbonyl-methylene” means a radical of the formula: ═CH—C(O)—O—C₁₋₄alkyl.

The term “C₁₋₄alkoxy-imino” means a radical of the formula: ═N—O—C₁₋₄alkyl.

The term “C₁₋₄alkyl-amino” or “diC₁₋₄alkyl-amino” means a radical of the formula: —NH—C₁₋₄alkyl or —N(C₁₋₄alkyl)₂, respectively.

The term “C₁₋₄alkyl-amino-C₁₋₄alkyl” or “diC₁₋₄alkyl-amino-C₁₋₄alkyl” means a radical of the formula: —C₁₋₄alkyl-NH—C₁₋₄alkyl or —C₁₋₄alkyl-N(C₁₋₄alkyl)₂, respectively.

The term “C₁₋₄alkyl-amino-C₁₋₄alkoxy-imino” or “diC₁₋₄alkyl-amino-C₁₋₄alkoxy-imino” means a radical of the formula: ═N—O—C₁₋₄alkyl-NH—C₁₋₄alkyl or ═N—O—C₁₋₄alkyl-N(C₁₋₄alkyl)₂, respectively.

The term “C₁₋₄alkyl-amino-C₁₋₄alkyl-amino” or “diC₁₋₄alkyl-amino-C₁₋₄alkyl-amino” means a radical of the formula: —NH—C₁₋₄alkyl-NH—C₁₋₄alkyl or —NH—C₁₋₄alkyl-N(C₁₋₄alkyl)₂, respectively.

The term “C₁₋₄alkyl-amino-C₁₋₄alkyl-amino-carbonyl-methylene” or “diC₁₋₄alkyl-amino-C₁₋₄alkyl-amino-carbonyl-methylene” means a radical of the formula: ═CH—C(O)—NH—C₁₋₄alkyl-NH—C₁₋₄alkyl or ═CH—C(O)—NH—C₁₋₄alkyl-N(C₁₋₄alkyl)₂, respectively.

The term “C₁₋₄alkyl-amino-C₁₋₄alkyl-carbonyl-amino” or “diC₁₋₄alkyl-amino-C₁₋₄alkyl-carbonyl-amino” means a radical of the formula: —NH—C(O)—C₁₋₄alkyl-NH—C₁₋₄alkyl or —NH—C(O)—C₁₋₄alkyl-N(C₁₋₄alkyl)₂, respectively.

The term “C₁₋₄alkyl-amino-carbonyl-methylene” or “diC₁₋₄alkyl-amino-carbonyl-methylene” means a radical of the formula: ═CH—C(O)—NH—C₁₋₄alkyl or ═CH—C(O)—N(C₁₋₄alkyl)₂, respectively.

The term “C₁₋₄alkyl-amino-imino” or “diC₁₋₄alkyl-amino-imino” means a radical of the formula: ═N—NH—C₁₋₄alkyl or ═N—N(C₁₋₄alkyl)₂, respectively.

The term “C₁₋₄alkyl-amino-sulfonyl-amino-carbonyl” or “diC₁₋₄alkyl-amino-sulfonyl-amino-carbonyl” means a radical of the formula: —C(O)—NH—SO₂—NH—C₁₋₄alkyl or —C(O)—NH—SO₂—N(C₁₋₄alkyl)₂, respectively.

The term “C₁₋₄alkyl-imino” means a radical of the formula: ═N—C₁₋₄alkyl.

The term “amino” means a radical of the formula: —NH₂.

The term “amino-C₁₋₄alkyl” means a radical of the formula: —C₁₋₄alkyl-NH₂.

The term “amino-C₁₋₄alkyl-amino” means a radical of the formula: —NH—C₁₋₄alkyl-NH₂.

The term “amino-C₁₋₄alkoxy” means a radical of the formula: —O—C₁₋₄alkyl-NH₂.

The term “amino-C₁₋₄alkoxy-imino” means a radical of the formula: ═N—O—C₁₋₄alkyl-NH₂.

The term “amino-carbonyl” means a radical of the formula: —C(O)—NH₂.

The term “amino-carbonyl-methylene” means a radical of the formula: ═CH—C(O)—NH₂.

The term “amino-C₁₋₄alkyl-amino-carbonyl-methylene” means a radical of the formula: ═CH—C(O)—NH—C₁₋₄alkyl-NH₂.

The term “amino-C₁₋₄alkyl-carbonyl-amino” means a radical of the formula: —NH—C(O)—C₁₋₄alkyl-NH₂.

The term “amino-imino” means a radical of the formula: ═N—NH₂.

The term “amino-sulfonyl-amino” means a radical of the formula: —NH—SO₂—NH₂.

The term “amino-sulfonyl-amino-carbonyl” means a radical of the formula: —C(O)—NH—SO₂—NH₂.

The term “aryl-oxy-imino” means a radical of the formula: ═N—O-aryl.

The term “carboxy” means a radical of the formula: —C(O)OH.

The term “carboxy-C₁₋₄alkoxy-imino” means a radical of the formula: ═N—O—C₁₋₄alkyl-C(O)OH.

The term “carboxy-methylene” means a radical of the formula: ═CH—C(O)OH.

The term “halogen” or “halo” means the group chloro, bromo, fluoro or iodo.

The term “heteroaryl-amino-imino” means a radical of the formula: ═N—NH-heteroaryl.

The term “heteroaryl-carbonyl-amino-imino” means a radical of the formula: ═N—NH—C(O)-heteroaryl.

The term “heterocyclyl-C₁₋₄alkyl-amino-carbonyl-methylene” means a radical of the formula: ═CH—C(O)—NH—C₁₋₄alkyl-heterocyclyl.

The term “hydroxy-C₁₋₄alkyl-amino-carbonyl-methylene” means a radical of the formula: ═CH—C(O)—NH—C₁₋₄alkyl-hydroxy, wherein C₁₋₄alkyl is substituted on one or more available carbon chain atoms with one or more hydroxy radicals when allowed by available valences.

The term “hydroxy-amino” means a radical of the formula: —NH-hydroxy.

The term “hydroxy-imino” means a radical of the formula: ═NH-hydroxy.

The term “phenyl-carbonyl-amino” means a radical of the formula: —NH—C(O)-phenyl.

The term “substituted” means the independent replacement of one or more hydrogen atoms within a radical with that amount of substitutents allowed by available valences.

In general, IUPAC nomenclature rules are used herein.

### Compound Forms

The term “about,” whether used explicitly or not in reference to a quantitative expression given herein, means that every quantity given herein qualified with the term or otherwise is meant to refer both to the actual given value and the approximation to such given value that would reasonably be inferred based on the ordinary skill in the art, including approximations due to experimental and/or measurement conditions for such given value.

The term “form” means, in reference to compounds of the present invention, such may exist as, without limitation, a salt, stereoisomer, tautomer, crystalline, polymorph, amorphous, solvate, hydrate, ester, prodrug or metabolite form. The present invention encompasses all such compound forms and mixtures thereof.

The term “isolated form” means, in reference to compounds of the present invention, such may exist in an essentially pure state such as, without limitation, an enantiomer, a racemic mixture, a geometric isomer (such as a cis or trans stereoisomer), a mixture of geometric isomers, and the like. The present invention encompasses all such compound forms and mixtures thereof.

The compounds of the invention may be present in the form of pharmaceutically acceptable salts. For use in medicines, the “pharmaceutically acceptable salts” of the compounds of this invention refer to non-toxic acidic/anionic or basic/cationic salt forms.

Suitable salt forms include acid addition salts which may, for example, be formed by mixing a solution of the compound according to the invention with a solution of an acid such as acetic acid, adipic acid, benzoic acid, carbonic acid, citric acid, fumaric acid, glycolic acid, hydrochloric acid, maleic acid, malonic acid, phosphoric acid, saccharinic acid, succinic acid, sulphuric acid, tartaric acid, trifluoroacetic acid and the like.

Furthermore when the compounds of the present invention carry an acidic moiety, suitable salts thereof may include alkali metal salts, e.g. sodium or potassium salts; alkaline earth metal salts, e.g. calcium or magnesium salts; and salts formed with suitable organic ligands, e.g. quaternary ammonium salts.

Thus, representative salts include the following: acetate, adipate, benzenesulfonate, benzoate, bicarbonate, bisulfate, bitartrate, borate, bromide, calcium, camsylate (or camphosulphonate), carbonate, chloride, clavulanate, citrate, dihydrochloride, edetate, fumarate, gluconate, glutamate, glyconate, hydrabamine, hydrobromine, hydrochloride, iodide, isothionate, lactate, malate, maleate, malonate, mandelate, mesylate, nitrate, oleate, pamoate, palmitate, phosphate/diphosphate, saccharinate, salicylate, stearate, sulfate, succinate, tartrate, tosylate, trichloroacetate, trifluoroacetate and the like.

Examples of salt forms of compounds representative of the present invention include the monohydrochloride salt.

During any of the processes for preparation of the compounds of the present invention, it may be necessary and/or desirable to protect sensitive or reactive groups on any of the molecules concerned. This may be achieved by means of conventional protecting groups, such as those described in Protective Groups in Organic Chemistry, ed. J. F. W. McOmie, Plenum Press, 1973; and T. W. Greene & P. G. M. Wuts, Protective Groups in Organic Synthesis, 3^{rd} Edition, John Wiley & Sons, 1999. The protecting groups may be removed at a convenient subsequent stage using methods known in the art. The scope of the present invention encompasses all such protected compound forms and mixtures thereof.

The invention includes compounds of various isomers and mixtures thereof. The term “isomer” refers to compounds that have the same composition and molecular weight but differ in physical and/or chemical properties. Such substances have the same number and kind of atoms but differ in structure. The structural difference may be in constitution (geometric isomers) or in an ability to rotate the plane of polarized light (optical isomers).

The term “stereoisomer” refers to isomers that have the same molecular formula and the same sequence of covalently bonded atoms but a different spatial orientation.

The term “optical isomer” means isomers of identical constitution that differ only in the spatial arrangement of their groups. Optical isomers rotate the plane of polarized light in different directions. The term “optical activity” means the degree to which an optical isomer rotates the plane of polarized light.

The term “racemate” or “racemic mixture” means an equimolar mixture of two enantiomeric species, wherein each of the isolated species rotates the plane of polarized light in the opposite direction such that the mixture is devoid of optical activity.

The term “enantiomer” means an isomer having a nonsuperimposable mirror image. The term “diastereomer” means stereoisomers that are not enantiomers.

The term “chiral” means a molecule which, in a given configuration, cannot be superimposed on its mirror image. This is in contrast to achiral molecules which can be superimposed on their mirror images.

The two distinct mirror image versions of the chiral molecule are also known as levo (left-handed), abbreviated L, or dextro (right-handed), abbreviated D, depending on which way they rotate polarized light. The symbols “R” and “S” represent the configuration of groups around a stereogenic carbon atom(s).

The term “geometric isomer” means isomers that differ in the orientation of substituent atoms in relationship to a carbon-carbon double bond, to a cycloalkyl ring, or to a bridged bicyclic system. Substituent atoms (other than hydrogen) on each side of a carbon-carbon double bond may be in an E or Z configuration. In the “E” configuration, the substituents are on opposite sides in relationship to the carbon-carbon double bond. In the “Z” configuration, the substituents are oriented on the same side in relationship to the carbon-carbon double bond. As illustrated by:

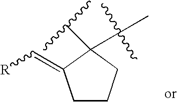

the wave line between the double bond and an R substituent for certain compounds of the present invention is intended to represent that the orientation of the R substituent atoms in relationship to the carbon-carbon double bond are not designated either E or Z. Accordingly, the illustrated bond line and orientation imply that the substituent atoms may be in either the E or Z configuration and that the isomers may be present in a mixture. All such configurations are intended to be included within the scope of the present invention.

Substituent atoms (other than hydrogen) attached to a ring system may be in a cis or trans configuration. In the “cis” configuration, the substituents are on the same side in relationship to the plane of the ring; in the “trans” configuration, the substituents are on opposite sides in relationship to the plane of the ring. Compounds having a mixture of “cis” and “trans” species are designated “cis/trans”.

Accordingly, as illustrated by:

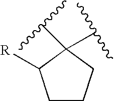

the line between the ring and an R substituent for certain compounds of the present invention is intended to represent that the orientation of substituent atoms in relationship to the chiral ring atom are not designated either cis or trans. Accordingly, the illustrated bond line and orientation implies that the R substituent atoms may be in either the cis or trans configuration and that the isomers may be present in a mixture. All such configurations are intended to be included within the scope of the present invention.

The isomeric descriptors (“R,” “S,” “E,” and “Z”) indicate atom configurations and are intended to be used as defined in the literature.

The compounds of the invention may be prepared as individual isomers by either isomer-specific synthesis or resolved from an isomeric mixture. Conventional resolution techniques include combining the free base (or free acid) of each isomer of an isomeric pair using an optically active acid (or base) to form an optically active salt (followed by fractional crystallization and regeneration of the free base), forming an ester or amide of each of the isomers of an isomeric pair by reaction with an appropriate chiral auxiliary (followed by fractional crystallization or chromatographic separation and removal of the chiral auxiliary), or separating an isomeric mixture of either an intermediate or a final product using various well known chromatographic methods.

Furthermore, compounds of the present invention may have one or more polymorph or amorphous crystalline forms and, as such, are intended to be included in the scope of the invention. In addition, some of the compounds may form solvates with water (i.e., hydrates) or common organic solvents (e.g., organic esters such as ethanolate and the like) and, as such, are also intended to be encompassed within the scope of this invention.

### Methods of Use

The present invention provides substituted spirobenzoazepanes compounds which are useful as dual or selective vasopressin receptor antagonists, inhibiting the binding of vasopressin to the V-1a, V-2 or V-1a and V-2 receptors.

The compounds of this invention also show functional activity by their ability to inhibit intracellular calcium mobilization and cyclic-AMP accumulation induced by arginine vasopressin (AVP) in transfected HEK-293 cells expressing human V-1a and V-2 receptors.

The instant compounds show the ability to block vasopressin binding to recombinant V-1a and/or V-2, and are therefore useful for treating conditions such as aggression, obsessive-compulsive disorders, hypertension, dysmenorrhea, hyponatremia, congestive heart failure, cardiac insufficiency, coronary vasospasm, cardiac ischemia, liver cirrhosis, renal vasospasm, renal failure, polycystic kidney disease, diabetic nephropathy, edema, ischemia, cerebral edema, cerebral ischemia, inner ear disorders, stroke, thrombosis, water retention, nephrotic syndrome, anxiety and central nervous injuries.

Embodiments of the present invention include a method for treating a vasopressin receptor mediated condition in a subject in need thereof comprising administering to the subject an effective amount of at least one compound of Formula (I).

Embodiments of the present invention include a method wherein the compound of Formula (I) is a dual or selective vasopressin receptor antagonist, and wherein the vasopressin receptor is selected from the V-1a, V-2 or V-1a and V-2 receptors.

Embodiments of the present invention include a use of the compound of Formula (I) in the manufacture of a medicament for treating a vasopressin receptor mediated condition.

Embodiments of the present invention include a use of the compound of Formula (I) as a medicine.

Embodiments of the present invention include a vasopressin receptor mediated condition selected from edema, ischemia, inner ear disorders, hypertension, congestive heart failure, cardiac insufficiency, coronary vasospasm, cardiac ischemia, liver cirrhosis, renal vasospasm, renal failure, polycystic kidney disease, diabetic nephropathy, hyponatremia, cerebral edema, cerebral ischemia, stroke, thrombosis, water retention, aggression, obsessive-compulsive disorders, dysmenorrhea, nephrotic syndrome, anxiety and central nervous injuries.

Embodiments of the present invention include a vasopressin receptor mediated condition selected from hypertension, congestive heart failure, cardiac insufficiency, diabetic nephropathy, dysmenorrhea, renal failure, hyponatremia or stroke.

Embodiments of the present invention include a vasopressin receptor mediated condition selected from congestive heart failure, diabetic nephropathy, dysmenorrhea, renal failure or hyponatremia.

The present invention includes a compound of Formula (I), or a form thereof, wherein the compound is labeled with a ligand for use as a marker, and wherein the ligand is a radioligand selected from deuterium, tritium and the like.

The term “administering,” with respect to the methods of the present invention, refers to a means for treating a condition as described herein with a compound of Formula (I) or a form thereof, which would obviously be included within the scope of the invention albeit not specifically disclosed for certain of said compounds.

Such methods include administering an effective amount of compound of Formula (I) or a form thereof at different times during the course of a therapy or concurrently in a combination form. Such methods further include administering an effective amount of said compound with one or more agents at different times during the course of a therapy or concurrently in a combination form.

The term “prodrug” means a compound of Formula (I) or a form thereof that is converted in vivo into a functional derivative form that may contribute to therapeutic biological activity, wherein the converted form may be: 1) a relatively active form; 2) a relatively inactive form; 3) a relatively less active form; or, 4) any form which results, directly or indirectly, from such in vivo conversions.

Prodrugs are useful when said compound may be either too toxic to administer systemically, absorbed poorly by the digestive tract or broken down by the body before it reaches its target. Conventional procedures for the selection and preparation of suitable prodrug derivatives are described in, for example, “Design of Prodrugs”, ed. H. Bundgaard, Elsevier, 1985.

The term “metabolite” means a prodrug form of a compound of Formula (I) or a form thereof converted by in vivo metabolism or a metabolic process to a relatively less active functional derivative of said compound.

The term “subject” as used herein, refers to a patient, such as an animal, a mammal or a human, who has been the object of treatment, observation or experiment and is at risk of (or susceptible to) developing a vasopressin mediated condition.

The term “effective amount” refers to that amount of a compound of Formula (I) or a form, pharmaceutical composition, medicine or medicament thereof that elicits the biological or medicinal response in a tissue system, animal or human, that is being sought by a researcher, veterinarian, medical doctor, or other clinician, which includes alleviation of the symptoms of the condition being treated.

The effective amount of said compound is from about 0.001 mg/kg/day to about 300 mg/kg/day.

The term “composition” refers to a product containing a compound of Formula (I) or a form thereof, such product comprising specified ingredients in specified amounts, as well as any product which results, directly or indirectly, from such combinations of the specified ingredients in the specified amounts.

The term “medicament” or “medicine” refers to a product containing a compound of Formula (I) or a form thereof. The present invention includes use of such a medicament for treating a vasopressin mediated condition.

The term “combination form” refers to the use of a combination product comprising a compound of Formula (I) or a form, pharmaceutical composition, medicine or medicament thereof and at least one therapeutic agent for treating a vasopressin mediated condition.

Advantageously, the effective amount of a combination product for treating a vasopressin mediated condition may be a reduced amount of either or both the compound or therapeutic agent compared to the effective amount of the compound or therapeutic agent otherwise recommended for treating thecondition. Therefore, it is contemplated that the compound is administered to the subject before, during or after the time the agent is administered.

The term “treating” refers, without limitation, to facilitating the eradication of, preventing, ameliorating or otherwise inhibiting the progression of or promoting stasis of a vasopression mediated condition.

The present invention includes a pharmaceutical composition comprising an admixture of a compound of Formula (I) or a form thereof and one or more pharmaceutically acceptable excipients.

The present invention includes a process for making a pharmaceutical composition, medicine or medicament comprising mixing a compound of Formula (I) or a form thereof and an optional pharmaceutically acceptable carrier. The present invention includes a pharmaceutical composition, medicine or medicament resulting from the process of mixing a compound of Formula (I) or a form thereof and an optional pharmaceutically acceptable carrier.

Said pharmaceutical composition, medicine or medicament may take a wide variety of forms to effectuate mode of administration, wherein the mode includes, and is not limited to, intravenous (both bolus and infusion), oral, nasal, transdermal, topical with or without occlusion, and via injection intraperitoneally, subcutaneously, intramuscularly, intratumorally, intracerebrally or intracranially. The composition, medicine or medicament may be in a dosage unit such as a tablet, pill, capsule, powder, granule, sterile parenteral solution or suspension, metered aerosol or liquid spray, drop, ampoule, auto-injector device or suppository for such administration modes.

Pharmaceutical compositions, medicines or medicaments suitable for oral administration include solid forms such as pills, tablets, caplets, capsules (each including immediate release, timed release and sustained release formulations), granules and powders; and, liquid forms such as solutions, syrups, elixirs, emulsions and suspensions. Forms useful for parenteral administration include sterile solutions, emulsions and suspensions. Alternatively, the pharmaceutical composition, medicine or medicament may be presented in a form suitable for once-weekly or once-monthly administration; for example, an insoluble salt of the active compound, such as the decanoate salt, may be adapted to provide a depot preparation for intramuscular injection.

The dosage form (tablet, capsule, powder, injection, suppository, teaspoonful and the like) containing the pharmaceutical composition, medicine or medicament contains an effective amount of the active ingredient necessary to be effective as described above.

An example of a contemplated effective amount for a pharmaceutical composition, medicine or medicament of the present invention may range from about 0.001 mg to about 300 mg/kg of body weight per day. In another example, the range is from about 0.003 to about 100 mg/kg of body weight per day. In another example, the range is from about 0.005 to about 15 mg/kg of body weight per day. The pharmaceutical composition, medicine or medicament may be administered according to a dosage regimen of from about 1 to about 5 times per day.

For oral administration, the pharmaceutical composition, medicine or medicament is preferably in the form of a tablet containing, e.g., 0.01, 0.05, 0.1, 0.5, 1.0, 2.5, 5.0, 10.0, 15.0, 25.0, 50.0, 100, 150, 200, 250 and 500 milligrams of a compound of Formula (I) or a form thereof for the symptomatic adjustment of the dosage to the patient to be treated. Optimal dosages will vary depending on factors associated with the particular patient being treated (e.g., age, weight, diet and time of administration), the severity of the condition being treated, the particular compound being used, the mode of administration and the strength of the preparation. The use of either daily administration or post-periodic dosing may be employed.

### Synthetic Methods

Representative compounds of the present invention can be synthesized in accordance with the general synthetic schemes described below and are illustrated more particularly in the specific synthetic examples that follow. The general schemes are offered by way of illustration; the invention should not be construed as being limited by the chemical reactions and conditions expressed. The methods for preparing the various starting materials used in the schemes and examples are well within the skill of persons versed in the art. No attempt has been made to optimize the yields obtained in any of the example reactions. One skilled in the art would know how to increase such yields through routine variations in reaction times, temperatures, solvents and/or reagents.

General: ¹H and ¹³C NMR spectra were measured on a Bruker AC-300 (300 MHz) spectrometer using tetramethylsilane and the deuterated solvent respectively as internal standards. Elemental analyses were obtained by Quantitative Technologies Inc. (Whitehouse, N.J.) and the results were within 0.4% of the calculated values unless otherwise mentioned. Melting points were determined in open capillary tubes with a Mel-Temp II apparatus (Laboratory Devices Inc.) and were uncorrected. Electrospray mass spectra (MS-ESI) were recorded in the positive mode on a Hewlett Packard 59987A spectrometer. High resolution mass spectra (HRMS) were obtained on a Micromass Autospec. E spectrometer by fast atom bombardment (FAB) technique.

| Abbreviation | Meaning |
|---|---|
| BOC | tert-butyloxycarbonyl |
| BOP | benzotriazol-1-yloxy-tris(dimethylamino)phosphonium |
| | hexafluorophosphate |
| Cpd | compound |
| DCE | dichloroethane |
| DCM | dichloromethane |
| DMAP | dimethylaminopyridine |
| DMF | N,N-dimethylformamide |
| DMSO | dimethyl sulfoxide |
| EDC | 1-(3-dimethylaminopropyl)-3-ethylcarbodiimide |
| | hydrochloride |
| ESI | Electrospray Ionization |
| Et₃N TEA | triethylamine |
| EtOAc | ethyl acetate |
| h/hr/hrs | hour(s) |
| HOBT | 1-hydroxybenzotriazole hydrate |
| HBTU | O-benzotriazol-1-yloxy-N,N,N′,N′-tetramethyluronium |
| | hexafluorophosphate |
| LiOH | lithium hydroxide |
| min | minute(s) |
| MS | mass spectroscopy |
| NMR | nuclear magnetic resonance spectroscopy |
| PG | protecting group |
| RT/rt | room temperature |
| THF | tetrahydrofuran |
| TLC | thin layer chromatography |
| Tos | p-toluenesulfonyl |

### General Synthetic Methods

Representative compounds of the present invention can be synthesized in accordance with the general synthetic methods described below and are illustrated more particularly in the schemes that follow. Since the schemes are illustrations, the invention should not be construed as being limited by the chemical reactions and conditions expressed. The preparation of the various starting materials used in the schemes is well within the skill of persons versed in the art. The substituents U, V, W, a, b, R₁ and R₂ for compounds of Formula (I) or a form thereof, represented in the schemes below, are as previously defined herein. Furthermore, where used in the following schemes and description, the substituents Q-Q₁₂ represent starting material and intermediate compound substituents as indicated in the description.

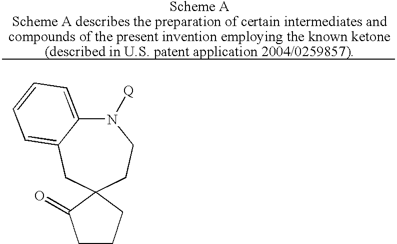

Intermediate compounds of formula A1 (wherein Q represents hydrogen) and A2 (wherein Q represents a protecting group such as p-tolenesulfonyl, arylsulfonyl or substituted arylsulfonyl) are used as starting materials for a variety of key intermediates.

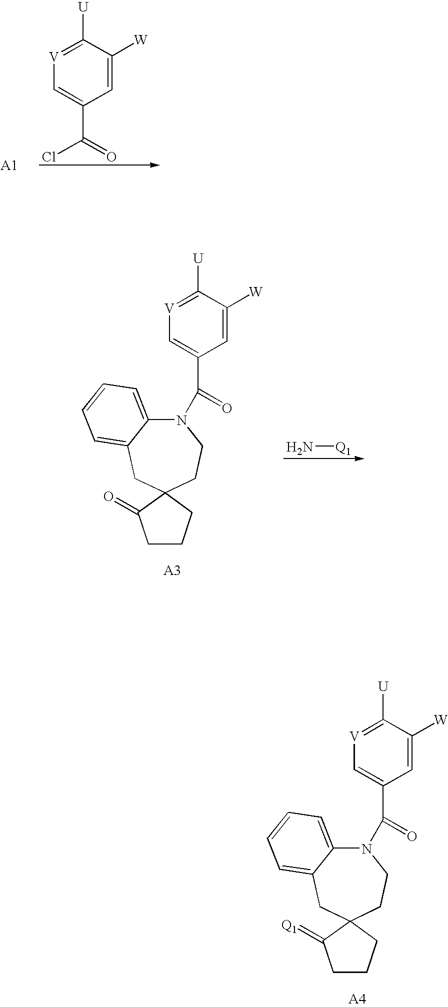

Thus, A1 can be treated with a variety of acid chlorides to give the intermediate A3, which is reacted further with various amines to afford target imines A4 (wherein Q, represents R₁ selected from C₁₋₄alkyl-imino, C₁₋₄alkoxy-imino, hydroxy-imino, amino-imino, C₁₋₄alkyl-amino-imino, diC₁₋₄alkyl-amino-imino, amino-C₁₋₄alkoxy-imino, C₁₋₄alkyl-amino-C₁₋₄alkoxy-imino, diC₁₋₄alkyl-amino-C₁₋₄alkoxy-imino, heteroaryl-amino-imino or heteroaryl-carbonyl-amino-imino, as previously defined).

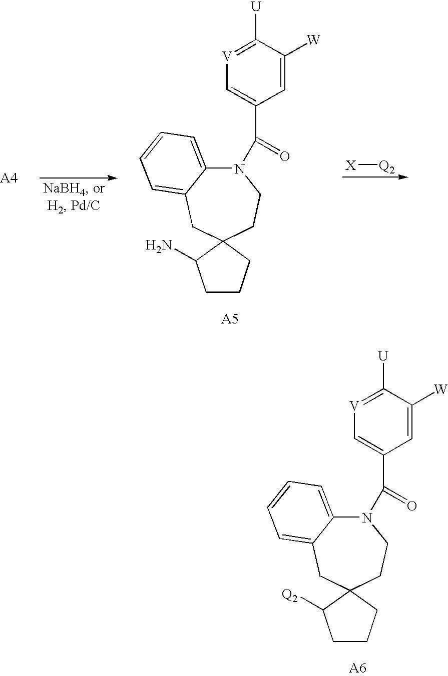

These A4 imines can be reduced to primary amines A5 (wherein R₁ is amino), which can be reacted with a variety of alkylating and acylating agents to give target compounds A6 (wherein Q₂ represents R₁ selected from C₁₋₄alkyl-amino, diC₁₋₄alkyl-amino, hydroxy-amino, amino-C₁₋₄alkyl-carbonyl-amino, C₁₋₄alkyl-amino-C₁₋₄alkyl-carbonyl-amino, diC₁₋₄alkyl-amino-C₁₋₄alkyl-carbonyl-amino or amino-sulfonyl-amino, as previously defined).

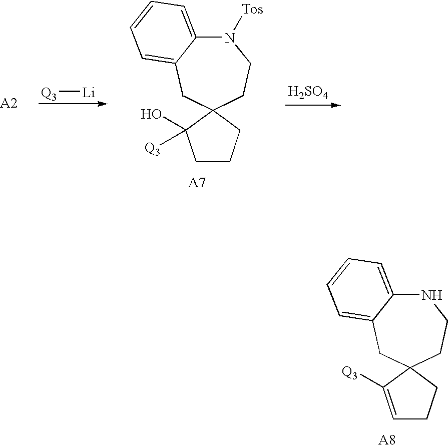

The ketone A2 may also be reacted with a variety of organometallic reagents, such as organolithium and Grignard reagents, to provide alcohols A7 (wherein Q₃ represents R₁ selected from heteroaryl, as previously defined). Compound A7 can then be deprotected and dehydrated to afford alkene A8 by treatment with strong acid, such as sulfuric acid.

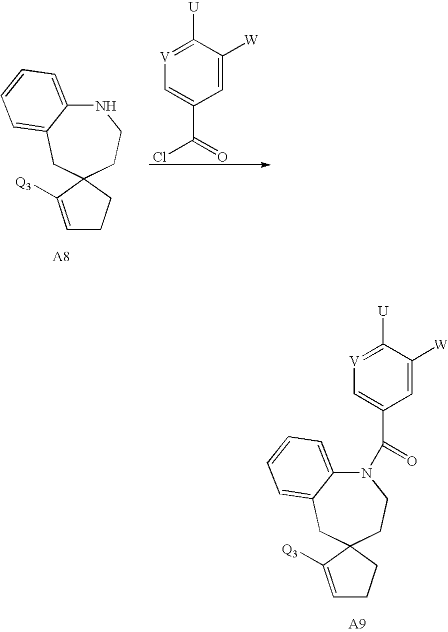

Subsequent reaction of A8 with a variety of acid chlorides can provide final target compounds A9.

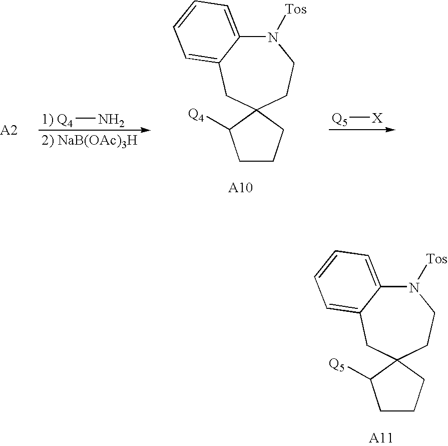

The ketone A2 may undergo reductive amination reactions with an appropriately substituted amine and sodium triacetoxyborohydride to prepare secondary amines A10 (wherein Q₄ represents R₁ selected from C₁₋₄alkyl-amino, hydroxy-amino, amino-C₁₋₄alkyl-carbonyl-amino, C₁₋₄alkyl-amino-C₁₋₄alkyl-carbonyl-amino, diC₁₋₄alkyl-amino-C₁₋₄alkyl-carbonyl-amino or amino-sulfonyl-amino, as previously defined)., which can be further alkylated to provide tertiary amines A11 (wherein Q₃ represents R₁ selected from diC₁₋₄alkyl-amino, as previously defined).

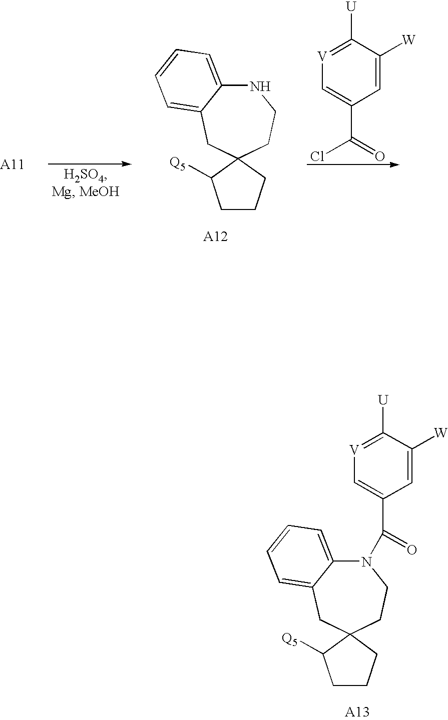

Intermediate A11 can be deprotected with either strong acid or magnesium in methanol to give A12, which can be reacted with an acid chloride to afford final target compounds A13.

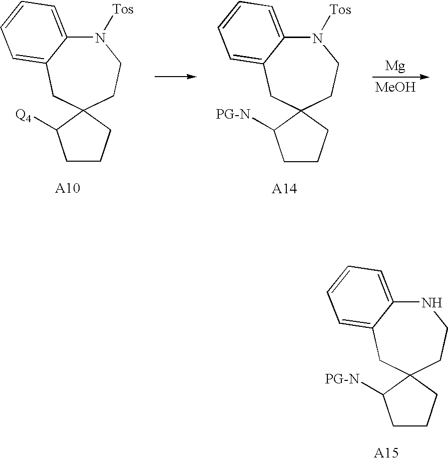

Alternatively, the free amino group of A10 (wherein Q₄ represents R₁ selected from C₁₋₄alkyl-amino, hydroxy-amino, amino-C₁₋₄alkyl-carbonyl-amino, C₁₋₄alkyl-amino-C₁₋₄alkyl-carbonyl-amino, diC₁₋₄alkyl-amino-C₁₋₄alkyl-carbonyl-amino or amino-sulfonyl-amino, as previously defined) can be protected (wherein PG represents a suitable protecting group) to give A14, which can subsequently be deprotected with magnesium in methanol or strong acid to furnish A15.

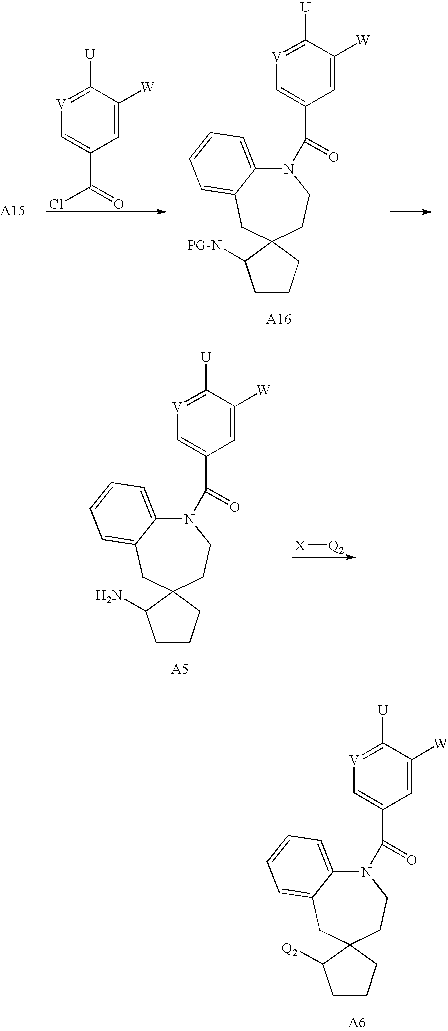

Reaction of A15 with an acid chloride A16 followed by removal of the protecting group and subsequent appropriate reactions gives the target primary amines A5 and secondary amines A6 (wherein Q₂ is as previously defined).

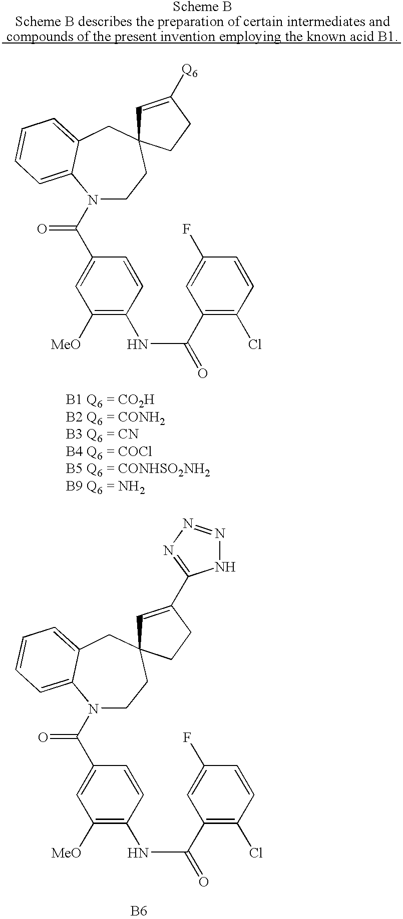

A compound of formula B1 (described in United States Patent Application 20040266752 and PCT Publication WO 05/037795) is used as starting material for a variety of key intermediates. Thus, B1 may be treated with a carbodiimide and ammonium hydroxide to afford the primary amide of formula B2. The compound of formula B2 may be dehydrated with cyanuric chloride to give the nitrile of formula B3, which may be reacted further with sodium azide to give a target tetrazole compound of formula B6. The acid B1 may also be converted to the acid chloride by heating with thionyl chloride to afford compound of formula B4, which may then be condensed with commercially available sulfamide to give the target acylsulfamide of formula B5.

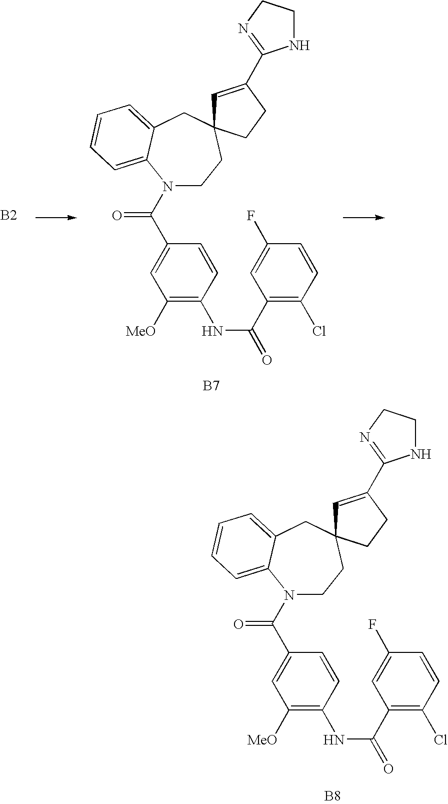

A compound of formula B2 may also be reacted with trimethyloxonium tetrafluoroborate to afford a reactive imidate intermediate, which reacts further with ethylenediamine to give target imidazoline of formula B7. This may then be oxidized with an appropriate oxidizing agent such as manganese dioxide to give the target imidazole of formula B8.

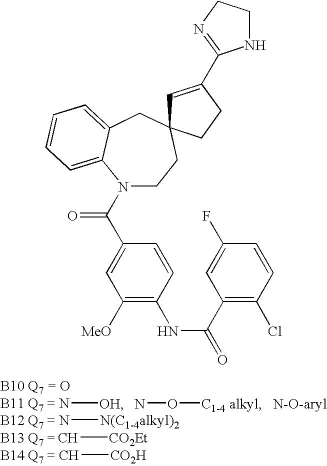

Compound B1 may also be reacted with hydrazoic acid and converted to the unstable enamine of formula B9, which rapidly hydrolyzed upon neutralization to afford the stable and more valuable intermediate ketone of formula B10. The ketone B10 may be condensed with hydroxylamines to afford the oximes of formula B11 or with hydrazines to afford the corresponding hydrazones of formula B12. The intermediate ketone B10 may also be used in the Wittig reaction, such as with triethyl-phosphonoacetate and sodium hydride to afford the alkenes B13. These may be easily hydrolyzed with a base, such as sodium hydroxide, and then acidified to give the acrylic acid derivatives B14.

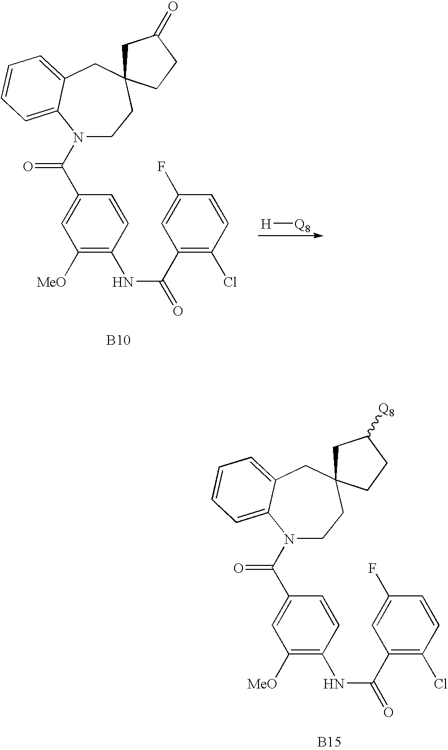

The ketone B10 may undergo reductive amination with an appropriately substituted amine and sodium triacetoxyborohydride to give the amines of formula B15 (wherein Q₈ generally represents R₂ mono or disubstituted amino, as previously defined).

The acid B14 may be reacted further and coupled to ammonia or a substituted amine using a coupling agent such as BOP or HBTU to afford the amides B16 (wherein Q₇ generally represents R₂ mono or disubstituted amino-carbonyl-methylene, as previously defined).

The following examples are offered by way of illustration; the invention should not be construed as being limited by the chemical reactions and conditions expressed.

### Example 1

### (R)-N-[3-methoxy-4-(2-chloro-5-fluoro-phenylcarbonyl)amino-phenylcarbonyl]-3-tetrazol-5-yl-spiro[cyclopent-2-ene-1,4′-benzo[b]azepane] (Cpd 11)

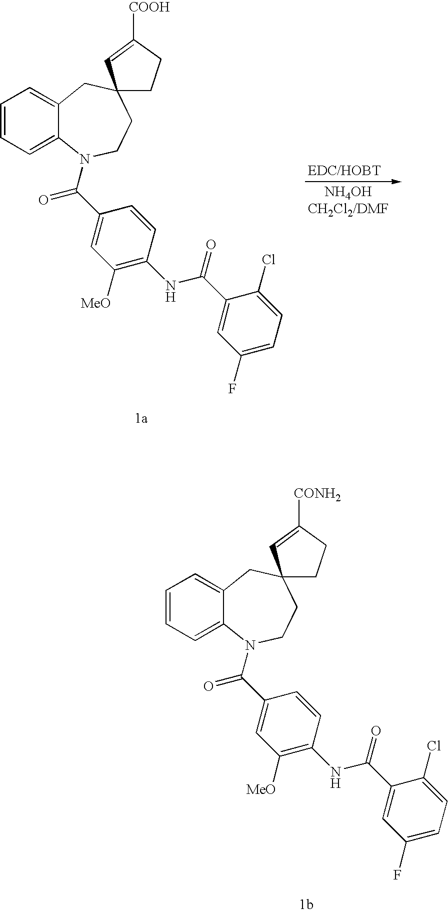

To a solution of Compound la (described in United States Patent Publication 20040266752 and PCT Publication WO 05/037795) (5.49 g, 0.010 mol) in DCM/DMF (5:1, 60 mL) was added N-(3-dimethylaminopropyl)-N′-ethylcarbodiimide hydrochloride (EDC) (2.11 g, 0.011 mol), and HOBT (1.53 g, 0.010 mol) and stirred at rt for 40 min. Ammonium hydroxide (0.90 mL, 0.0133 mol) was added in dropwise over about 5 min and reaction stirred for 6 h. The reaction was poured into water (75 mL) and extracted with DCM. The DCM was washed with saturated NaHCO₃, twice with brine, dried (Na₂SO₄), and evaporated in vacuo to an oil, which was purified by flash column chromatography eluting with EtOAc/MeOH (10:1) on silica gel to give Compound 1b as a white flakey solid (4.63 g) containing about 1 mole of DMF: 1H NMR (CDCl₃) δ 8.65(s, 1H), 8.25 (d, J=8.4 Hz, 1H), 8.01 (s, DMF, 1H), 7.5 (m, 1H), 7.4 (m, 1H), 7.3-7.0 (m, 4H), 6.95 (s, 1H), 6.7 (m, 2H), 6.49 (s, 0.50H), 6.25 (s, 0.50H), 5.6 (m, 2H), 4.9 (m, 1H), 3.73 (s, 3H), 3.33 (d, J=13.4 Hz, 1H), 3.1 (m, 1H), 2.96/2.88 (2 s, DMF), 2.7 (m, 3H), 2.1 (m, 2H), 1.75 (m, 2H). MS (ESI) m/z 548 (MH)⁺.

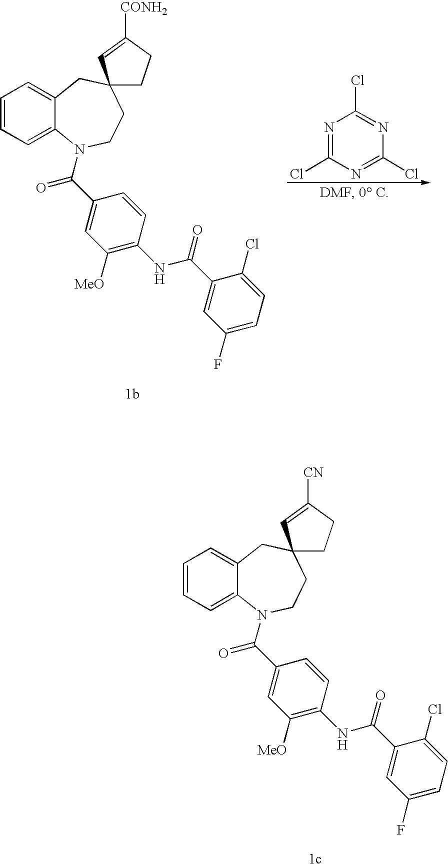

Compound 1b (500 mg, 0.91 mmol) in DMF (5 mL) was cooled to 0° C. and cyanuric chloride (120 mg, 0.65 mmol) was added in and reaction was stirred at rt for 3 h. The solution was partitioned between water and DCM, and the DCM solution was washed twice with brine, dried (Na₂SO₄) and evaporated in vacuo to give an oily product Compound 1c (0.47 g): ¹H NMR (CDCl₃) δ 8.66 (s, 1H), 8.25 (d, J=8.4 Hz, 1H), 8.01 (s, DMF), 7.5 (m, 1H), 7.4 (m, 1H), 7.3-6.9 (m, 5H), 6.7 (m, 2H), 6.6 (s, 0.50H), 6.3 (s, 0.50H), 4.9 (m, 1H), 3.74 (s, 3H), 3.36 (d, J=13.5 Hz, 1H), 3.1 (m, 1H), 2.96/2.87 (2 s, DMF), 2.7 (m, 3H), 2.1 (m, 2H), 1.75 (m, 2H). MS (ESI) m/z 530 (MH)⁺.

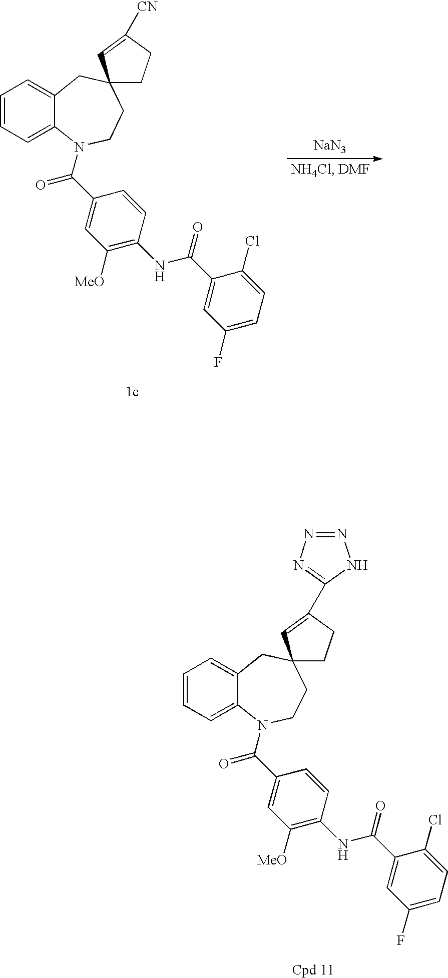

Compound 1c (380 mg, 0.72 mmol) was combined with sodium azide (150 mg, 2.3 mmol) and ammonium chloride (90 mg, 1.68 mmol) in DMF (8 mL) and heated to 125° C. for 15 h. The reaction was cooled to room temperature and partitioned between DCM, water and 1N H HCl to pH=1. The organic solution was separated and washed twice with brine, dried (Na₂SO₄) and evaporated in vacuo to a tan solid, which was purified by flash column chromatography on silical gel eluting with DCM/MeOH (5:1) to afford Compound 11 (100 mg): ¹H NMR (CDCl₃) δ 8.70 (s, 1H), 8.23 (d, J=8.5 Hz, 1H), 8.01 (s, DMF), 7.5-7.3 (m, 2H), 7.3-6.9 (m, 5H), 6.73 (m, 1H), 6.5 (m, 2H), 4.96 (m, 1H), 3.88 (s, 3H), 3.29 (d, J=14.0 Hz, 1H), 3.2-3.0 (m, 3H), 2.96/2.87 (2 s, DMF), 2.62 (d, J=13.6 Hz, 1H), 2.1 (m, 2H), 1.75 (m, 2H); MS (ESI) m/z 573 (MH)⁺.

### Example 2

### (R)-N-[3-methoxy-4-(2-chloro-5-fluoro-phenylcarbonyl)amino-phenylcarbonyl]-3-(4,5-dihydro-1H-imidazol-2-yl)-spiro[cyclopent-2-ene-1,4′-benzo[b]azepane] (Cpd 12)

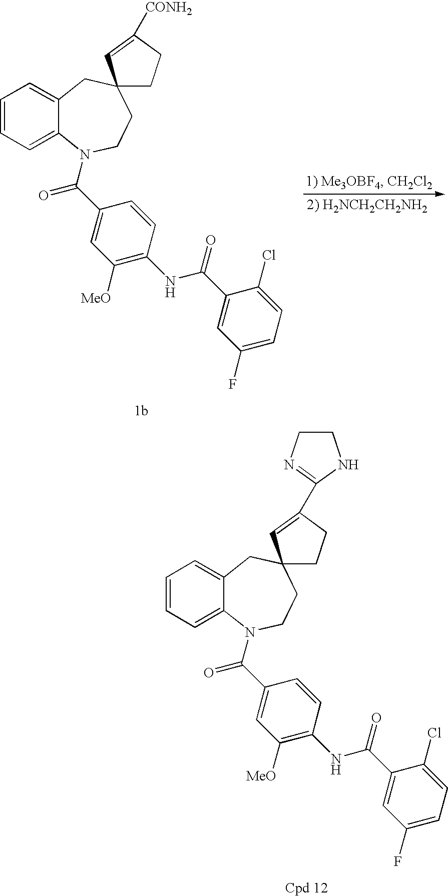

Trimethyloxonium tetrafluoroborate (222 mg, 1.50 mmol) was added to Compound 1b (547 mg, 1.0 mmol) in DCM (15 mL) and stirred at rt for 2.5 h. Ethylenediamine (66 mg, 1.10 mmol) was added in dropwise and the solution was stirred at rt for 16 h. The reaction was diluted with CHCl₃ and the organic layer was washed once with saturated NaHCO₃, once with water, once with brine, dried (Na₂SO₄) and evaporated in vacuo to a white solid. The solid was purified via flash column (EtOAc/MeOH/NH₄OH 40:4:1) to give Compound 12 as a white solid (260 mg): ¹H NMR (CDCl₃) δ 8.65 (s, 1H), 8.26 (d, J=8.4 Hz, 1H), 7.5-7.39 (m, 2H), 7.25-7.10 (m, 3H), 7.05-6.90 (m, 2H), 6.8-6.65 (m, 2H), 6.14 (s, 0.5H), 5.84 (s, 0.5H), 4.90 (m, 1H), 3.8-3.5 (m, 4H), 3.72 (s, 3H), 3.35 (m, 1H), 3.05 (m, 1H), 2.75 (m, 3H), 2.1 (m, 2H), 1.75 (m, 2H); MS (ESI) m/z 573 (MH)⁺.

### Example 3

### (R)-N-[3-methoxy-4-(2-chloro-5-fluoro-phenylcarbonyl)amino-phenylcarbonyl]-3-(1H-imidazol-2-yl)-spiro[cyclopent-2-ene-1,4′-benzo[b]azepane] (Cpd 14)

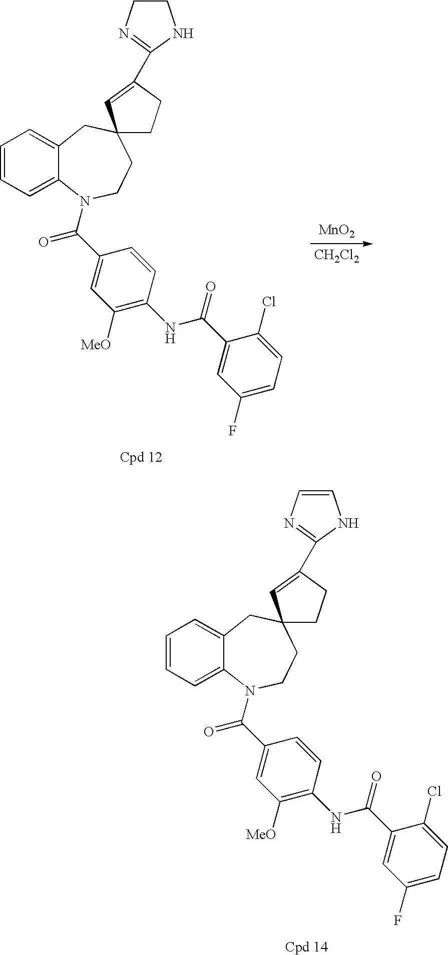

Compound 12 (140 mg, 0.24 mmol) was dissolved in DCM (12 mL) and activated manganese dioxide 85% (500 mg, 5.7 mmol) was added and the reaction was stirred at rt for 16 h. An additional portion of manganese dioxide (500 mg) was added and after another 24 h another portion (500 mg) was added and stirred at rt for additional 24h. The reaction was diluted with chloroform containing MeOH (10%) then filtered to remove the manganese dioxide. The filtrate was evaporated in vacuo to a residue, which was purified by flash column chromatography on silica gel EtOAc/MeOH/NH₄OH (40:4:1) to give Compound 14 as a white solid (48 mg): ¹H NMR (CDCl₃) δ 8.67 (s, 1H), 8.26 (d, J=8.4 Hz, 1H), 7.5-7.38 (m, 2H), 7.25-6.90 (m, 7H), 6.80-6.60 (m, 2H), 6.12 (s, 0.6H), 5.95 (s, 0.4H), 4.85 (m, 1H), 3.67 (s, 3H), 3.35 (m, 1H), 3.1-2.80 (m, 3H), 2.70 (m, 1H), 2.1 (m, 2H), 1.75 (m, 2H): MS (ESI) m/z 571 (MH)⁺.

### Example 4

### (R)-N-[3-methoxy-4-(2-chloro-5-fluoro-phenylcarbonyl)amino-phenylcarbonyl]-3-[(dimethylaminosulfonyl)aminocarbonyl]-spiro[cyclopent-2-ene-1,4′-benzo[b]azepane] (Cpd 36)

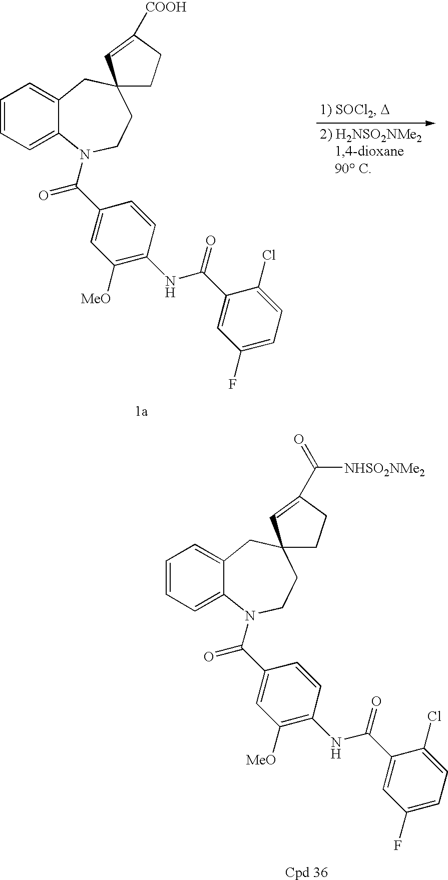

Compound 1a (550 mg, 1.0 mmol) was combined with thionyl chloride (7 mL) and refluxed for 1 h, and then evaporated in vacuo to afford a yellow solid. This was dissolved in dry 1,4-dioxane (7 mL) and dimethylsulfamide (500 mg, 4.0 mmol) was added and reaction heated to 90° C. for 4 h. An additional amount of sulfamide (375 mg, 3.0 mmol) was added and reaction maintained at 90° C. for 9 h and then cooled to rt. The reaction was diluted with 1,4-dioxane and filtered of solid and the filtrate was evaporated in vacuo to an oil, which was dissolved in DCM and washed with water, brine and dried (Na₂SO₄) and evaporated in vacuo to crude solid product (700 mg). This was purified by flash column chromatography on silica gel (EtOAc) to give Compound 36 as a white solid (170 mg): ¹H NMR (CDCl₃) δ 8.67(s, 1H), 8.25 (d, J=8.4 Hz, 1H), 7.5-7.4 (m, 2H), 7.2-6.9 (m, 5H), 6.70 (m, 2.5H), 6.33 (s, 0.50H), 4.9 (m, 1H), 3.74 (s, 3H), 3.34 (dd, J=12.9, 13.2 Hz, 1H), 3.1 (m, 1H), 3.01/2.97 (2 s, 6H), 2.8-2.55 (m, 3H), 2.1 (m, 2H), 1.75 (m, 2H); MS (ESI) m/z 655 (MH)⁺.

Using the procedure of Example 4, other compounds representative of the present invention were prepared:

| Cpd | Name and Data |
|---|---|
| 13 | (R)-3-aminosulfonylaminocarbonyl-N-[3-methoxy-4-(2-chloro-5-fluoro- |
| | phenylcarbonyl)amino-phenylcarbonyl]-spiro[cyclopent-2-ene-1,4′- |
| | benzo[b]azepane] |
| | ¹H NMR (CDCl₃) δ 8.70 (s, 1H), 8.25 (m, 1H), 7.5-7.35 (m, 2H), 7.25-6.9 (m, 5H), |
| | 6.8-6.6 (m, 2.5H), 6.43 (s, 0.5H), 5.7 (bs, 1H), 4.8 (m, 1H), 4.35 (bs, |
| | 2H), 3.75 (s, 3H), 3.4-3.0 (m, 2H), 2.8-2.5 (m, 3H), 2.1-1.9 (m, 1H), |
| | 1.8-1.5 (m, 3H); MS (ESI) m/z: 627 (MH)⁺. |
| 52 | (R)-3-aminosulfonylaminocarbonyl-N-[4-(2-chloro-5-fluoro- |
| | phenylcarbonyl)amino-phenylcarbonyl]-spiro[cyclopent-2-ene-1,4′- |
| | benzo[b]azepane] |
| | ¹H NMR (CD₃OD) δ 7.53-7.48 (m, 3H), 7.33-7.14 (m, 6H), 7.05-7.00 (t, |
| | J = 7.4 Hz, 1H), 6.74-6.71 (m, 1.6H), 6.46 (s, 0.4H), 4.99-4.79 (m, 1H), |
| | 3.24-2.99 (m, 2H), 2.83-2.56 (m, 3H), 2.16-1.93 (m, 2H), 1.80-1.62 (m, |
| | 2H); MS (ESI) m/z: 597 (MH)⁺. |

### Example 5

### (R)-N-[3-methoxy-4-(2-chloro-5-fluoro-phenylcarbonyl)amino-phenylcarbonyl]-3-oxo-spiro[cyclopentane-1,4′-benzo[b]azepane] (Cpd 53)

### (R)-N-[3-methoxy-4-(2-chloro-5-fluoro-phenylcarbonyl)amino-phenylcarbonyl]-3-hydroxyimino-spiro[cyclopentane-1,4′-benzo[b]azepane] (Cpd 15)

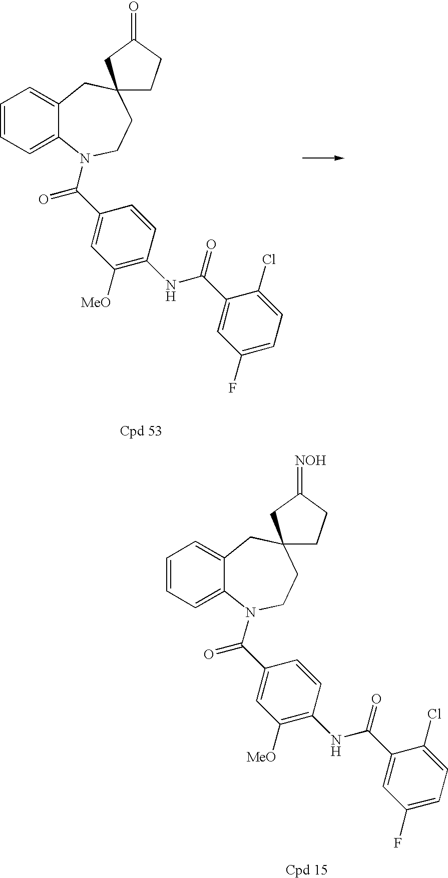

Sulfuric acid (95-98%, 5 mL) was added to Compound 1a (548 mg, 1.0 mmol) in chloroform (10 mL) and stirred vigorously at 50° C. as sodium azide (65 mg, 1.0 mmol) was added portionwise over 20 min. After 3 h, the reaction was cooled to rt and the organic layer was drawn off. The acid layer was then added dropwise to 3N NaOH (75 mL) with icebath cooling over the next 20 min. The aqueous solution was extracted with chloroform 3 times, and the combined organic solution was washed with brine once, dried (Na₂SO₄) and evaporated in vacuo to give Compound 53 as a white solid (550 mg): ¹H NMR (CDCl₃) δ 8.65(s, 1H), 8.25 (d, J=8.4 Hz, 1H), 7.55-7.4 (m, 2H), 7.2-6.95 (m, 5H), 6.70 (m, 2H), 4.95 (m, 1H), 3.74 (s, 3H), 3.30 (d, J=13.8 Hz, 1H), 2.96 (m, 1H), 2.65 (m, 1H), 2.5-2.3 (m, 3H), 2.25-2.0 (m, 3H), 1.8 (m, 2H); MS (ESI) m/z 521 (MH)⁺.

Compound 53 (580 mg, 1.1 mmol) was combined in ethanol (25 mL) with pyridine (11 mL) and hydroxylamine hydrochloride (380 mg, 5.5 mmol) and stirred at rt for 1 h. The solution was evaporated in vacuo to a solid, which was partitioned between chloroform and saturated NaHCO₃. The organic solution was washed once with saturated NaHCO₃, once with brine, dried (Na₂SO₄) and evaporated in vacuo to a white solid. This was purified by flash column chromatography on silica gel (EtOAc/MeOH 20:1) and solid was dissolved in EtOAc, dried (Na₂SO₄) and evaporated in vacuo to give Compound 15 as a white solid (340 mg): ¹H NMR (CDCl₃) δ 8.65(s, 1H), 8.26 (d, J=8.3 Hz, 1H), 7.55-7.4 (m, 2H), 7.25-6.90 (m, 6H), 6.70 (m, 2H), 4.90 (m, 1H), 3.73 (s, 3H), 3.25 (d, 1H), 2.95 (m, 1H), 2.8-2.4 (m, 4H), 2.3-2.0 (m, 2H), 1.86 (m, 1.8-1.4 (m, 2H); MS (ESI) m/z 536 (MH)⁺.

Using the procedure of Example 5, other compounds representative of the present invention were prepared:

| Cpd | Name and Data |
|---|---|
| 22 | (R)—N-[3-methoxy-4-(2-chloro-5-fluoro-phenylcarbonyl)amino- |
| | phenylcarbonyl]-3-methoxyimino-spiro[cyclopentane-1,4′-benzo[b]azepane] |
| | ¹HNMR (CDCl₃) δ 8.65 (s, 1H), 8.25 (d, J = 8.8 Hz, 1H), 7.61-7.39 (m, 2H), |
| | 7.20-7.10 (m, 3H), 7.09-6.92 (m, 2H), 6.74-6.67 (m, 2H), 4.87 (m, 1H), |
| | 3.80-3.72 (m, 3H), 3.65 (s, 3H), 3.47-3.38 (m, 1H), 3.25-2.90 (m, 1H), |
| | 2.62-2.31 (m, 5H), 2.24-2.09 (m, 2H), 1.87-1.64 (m, 2H); MS (ESI) m/z: 550 (MH)⁺. |
| 24 | (R)—N-[3-methoxy-4-(2-chloro-5-fluoro-phenylcarbonyl)amino- |
| | phenylcarbonyl]-3-carboxymethoxyimino-spiro[cyclopentane-1,4′- |
| | benzo[b]azepane] |
| | ¹H NMR (CDCl₃) δ 8.67 (s, 1H), 8.25 (d, J = 8.3 Hz, 1H), 7.5-7.39 (m, 2H), |
| | 7.25-7.1 (m, 3H), 7.1-6.9 (m, 2H), 6.75-6.65 (m, 2H), 5.0-4.8 (m, 1H), |
| | 4.7-4.5 (m, 2H), 4.2 (bs, 3H), 3.73 (s, 3H), 3.6-3.4 (m, 1H), 3.3-3.15 (m, 1H), |
| | 3.05-2.9 (m, 1H), 2.8-2.4 (m, 4H), 2.35-2.0 (m, 2H), 1.95-1.8 (m, 3H), |
| | 1.5-1.4 (m, 1H); MS (ESI) m/z: 594 (MH)⁺. |
| 27 | (R)—N-[3-methoxy-4-(2-chloro-5-fluoro-phenylcarbonyl)amino- |
| | phenylcarbonyl]-3-dimethylaminoimino-spiro[cyclopentane-1,4′- |
| | benzo[b]azepane] |
| | ¹HNMR (CDCl₃) δ 8.59 (s, 1H), 8.19 (d, J = 8.3 Hz, 1H), 7.60-7.32 (m, 2H), |
| | 7.14-7.09 (m, 3H), 7.08-6.88 (m, 2H), 6.62-6.59 (m, 2H), 4.85 (m, 1H), |
| | 3.67 (s, 3H), 3.45-3.12 (m, 1H), 3.08-2.77 (m, 1H), 2.61-2.55 (m, 1H), |
| | 2.45-2.24 (m, 3H), 2.08-1.99 (m, 3H), 1.84-1.68 (m, 2H), 1.5 (s, 6H); MS (ESI) m/z: |
| | 563 (MH)⁺. |

### Example 6

### (R)-N-[3-methoxy-4-(2-chloro-5-fluoro-phenylcarbonyl)amino-phenylcarbonyl]-3-carboxymethylene-spiro[cyclopentane-1,4′-benzo[b]azepane] (Cpd 16)

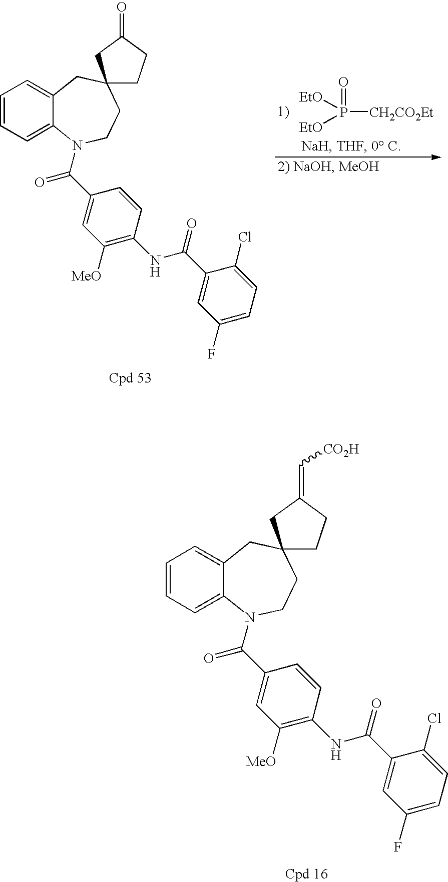

Triethylphosphono acetate (538 mg, 2.40 mmol) was added to NaH (67 mg, 2.65 mmol) stirring in THF (3.0 mL) at 0° C. and stirred at rt for 1 h. Compound 53 (410 mg, 0.80 mmol) in THF (5 mL) was added in dropwise over 5 min and the reaction was stirred at rt for 16 h. The reaction was partitioned between chloroform and saturated NaHCO₃, and the organic layer was washed once with brine, dried (Na₂SO₄) and evaporated in vacuo to provide an ester intermediate (730 mg). The intermediate was purified by flash column chromatography on silica gel (EtOAc/hexanes 5:3) to afford a white solid (230 mg): MS (ESI) m/z 591 (MH)⁺.

A portion of the intermediate (220 mg, 0.37 mmol) in methanol (10 mL) was combined with 1N NaOH (3.0 mL) and stirred at rt for 20 h. The solution was ice-bath cooled and 2N HCl was added to pH=3.0 and evaporated in vacuo to provide a white solid. The solid was partitioned between dilute HCl and chloroform, and the organic solution was washed once with water, once with brine, dried (Na₂SO₄) and evaporated in vacuo to give Compound 16 as a white flakey solid (200 mg): ¹H NMR (CDCl₃) δ 8.65(s, 1H), 8.25 (d, J=8.4 Hz, 1H), 7.55-7.4 (m, 2H), 7.2-6.95 (m, 5H), 6.70 (m, 2H), 4.95 (m, 1H), 3.74 (s, 3H), 3.30 (d, J=13.8 Hz, 1H), 2.96 (m, 1H), 2.65 (m, 1H), 2.5-2.3 (m, 3H), 2.25-2.0 (m, 3H), 1.8 (m, 2H); MS (ESI) m/z 521 (MH)⁺.

Using the procedure of Example 6, other compounds representative of the present invention were prepared:

| Cpd | Name and Data |
|---|---|
| 26 | (R)—N-[3-methoxy-4-(2-chloro-5-fluoro-phenylcarbonyl)amino- |
| | phenylcarbonyl]-3-methoxycarbonylmethylene-spiro[cyclopentane-1,4′- |
| | benzo[b]azepane] |
| | ¹HNMR (CDCl₃) δ 8.64 (s, 1H), 8.25 (d, J = 8.5 Hz, 1H), 7.60-7.32 (m, 2H), |
| | 7.19-7.12 (m, 3H), 7.09-6.93 (m, 2H), 6.75-6.65 (m, 2H), 5.89-5.83 (m, 1H), |
| | 4.90 (m, 1H), 3.72 (d, 6H), 3.23-3.13 (m, 1H), 2.98-2.91 (m, 2H), |
| | 2.76-2.51 (m, 3H), 2.11-2.04 (m, 1H), 1.93-1.81 (m, 1H), 1.78-1.53 (m, 3H); MS (ESI) |
| | m/z: 577 (MH)⁺. |

### Example 6a

### (R)-N-[3-methoxy-4-(2-chloro-5-fluoro-phenylcarbonyl)amino-phenylcarbonyl]-3-(2-dimethylamino-ethyl)aminocarbonylmethylene-spiro[cyclopentane-1,4′-benzo[b]azepane] (Cpd 31)

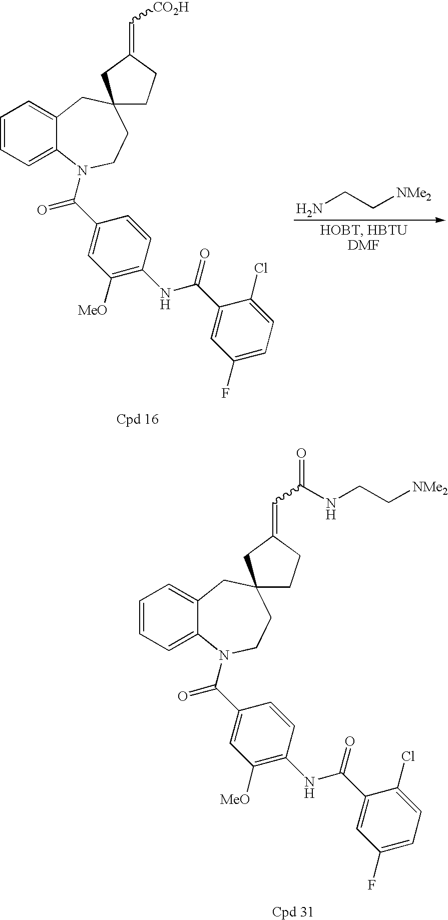

Compound 16 (50 mg, 0.089 mmol) was combined in DMF (2 mL) with N,N-dimethylethylene diamine (0.020 mL, 0.18 mmol), diisopropylethylamine (0.063 mL, 0.36 mmol), HOBT (24.3 mg, 0.18 mmol) and HBTU (68 mg, 0.18 mmol) and stirred overnight at rt. The reaction was diluted with chloroform and washed twice with water, once with saturated NaHCO₃, once with brine, dried (Na₂SO₄), evaporated in vacuo and the oil was purified by flash column chromatography on silica gel (DCM/MeOH/NH₄OH, 97:3:0.4) to afford Compound 31 (34.8 mg): ¹HNMR (CDCl₃) δ 8.64 (s, 1H), 8.25 (d, J=8.2 Hz, 1H), 7.50-7.39 (m, 2H), 7.19-7.01 (m, 3H), 6.98-6.95 (m, 2H), 6.75-6.65 (m, 2H), 6.38-6.28 (m, 1H), 4.82 (m, 1H), 3.72 (s, 3H), 3.34-3.27 (m, 2H), 3.07-3.00 (m, 2H), 2.64-2.59 (m, 1H), 2.46-2.40 (m, 3H), 2.25-2.22 (m, 6H), 2.18-1.94 (m, 2H), 1.66-1.52 (m, 6H); MS (ESI) m/z 633 (MH)⁺.

Using the procedure of Example 6a, other compounds representative of the present invention were prepared:

| Cpd | Name and Data |
|---|---|
| 28 | (R)—N-[3-methoxy-4-(2-chloro-5-fluoro-phenylcarbonyl)amino- |
| | phenylcarbonyl]-3-(2-morpholin-4-yl-ethyl)aminocarbonylmethylene- |
| | spiro[cyclopentane-1,4′-benzo[b]azepane] |
| | ¹HNMR (CDCl₃) δ 8.64 (s, 1H), 8.25 (d, J = 8.1 Hz, 1H), 7.61-7.39 (m, 2H), |
| | 7.21-7.12 (m, 3H), 7.10-6.94 (m, 2H), 6.75-6.65 (m, 2H), 6.18-6.09 (m, 1H), |
| | 5.62 (bs, 1H), 4.85 (m, 1H), 3.71 (d, 7H), 3.40-3.27 (m, 3H), 3.08-2.96 (m, |
| | 2H), 2.59-2.47 (m, 9H), 2.17-1.96 (m, 2H), 1.84-1.51 (m, 3H); MS (ESI) m/z: |
| | 675 (MH)⁺. |
| 29 | (R)—N-[3-methoxy-4-(2-chloro-5-fluoro-phenylcarbonyl)amino- |
| | phenylcarbonyl]-3-(2-hydroxy-ethyl)aminocarbonylmethylene- |
| | spiro[cyclopentane-1,4′-benzo[b]azepane] |
| | ¹HNMR (CDCl₃) δ 8.64 (s, 1H), 8.25 (d, J = 7.9 Hz, 1H), 7.61-7.38 (m, 2H), |
| | 7.26-7.12 (m, 3H), 6.98-6.92 (m, 2H), 6.75-6.66 (m, 2H), 6.18-6.15 (m, 1H), |
| | 5.61 (bs, 1H), 4.82 (m, 1H), 3.71 (s, 5H), 3.46-3.26 (m, 3H), 3.19-3.01 (m, |
| | 3H), 2.70-2.42 (m, 4H), 2.08-1.94 (m, 2H), 1.66-1.48 (m, 2H); MS (ESI) m/z: |
| | 606 (MH)⁺. |
| 30 | (R)—N-[3-methoxy-4-(2-chloro-5-fluoro-phenylcarbonyl)amino- |
| | phenylcarbonyl]-3-methylaminocarbonylmethylene-spiro[cyclopentane-1,4′- |
| | benzo[b]azepane] |
| | ¹HNMR (CDCl₃) δ 8.64 (s, 1H), 8.25 (d, J = 8.6 Hz, 1H), 7.50-7.39 (m, 2H), |
| | 7.23-7.10 (m, 3H), 6.98-6.93 (m, 2H), 6.84-6.67 (m, 2H), 5.60 (bd s, 2H), |
| | 4.82 (m, 1H), 3.72 (s, 3H), 3.47-3.26 (m, 3H), 3.19-2.98 (m, 2H), |
| | 2.86-2.80 (m, 4H), 2.70-2.41 (m, 3H), 2.14-1.94 (m, 2H); MS (ESI) m/z: 576 (MH)⁺. |
| 32 | (R)-3-aminocarbonylmethylene-N-[3-methoxy-4-(2-chloro-5-fluoro- |
| | phenylcarbonyl)amino-phenylcarbonyl]-spiro[cyclopentane-1,4′- |
| | benzo[b]azepane] |
| | ¹HNMR (CDCl₃) δ 8.64 (s, 1H), 8.25 (d, J = 8.2 Hz, 1H), 7.50-7.38 (m, 2H), |
| | 7.22-7.10 (m, 3H), 7.01-6.93 (m, 2H), 6.75-6.67 (m, 2H), 5.63-5.54 (m, 2H), |
| | 5.38-5.32 (m, 1H), 4.82 (m, 1H), 3.72 (s, 3H), 3.31-3.27 (m, 1H), |
| | 3.09-3.01 (m, 3H), 2.69-2.42 (m, 3H), 2.09-1.96 (m, 2H), 1.71-1.43 (m, 2H); MS (ESI) |
| | m/z: 562 (MH)⁺. |

### Example 7

### 2-aminoimino-N-[4-(2-chloro-5-fluoro-phenylcarbonyl)amino-phenylcarbonyl]-spiro[cyclopentane-1,4′-benzo[b]azepane] (Cpd 1)

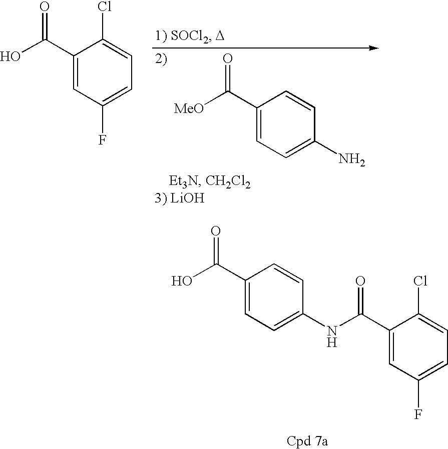

### Step A. 4-(2-chloro-5-fluoro-benzoylamino)-benzoic acid

A solution of 2-chloro-5-fluoro-benzoic acid (5.00 g, 28.6 mmol; CAS 2252-50-8) in thionyl chloride (10.0 mL, 137 mmol) was refluxed under an argon atmosphere for 1 hour, cooled to room temperature, concentrated in vacuo, and dissolved in 30 mL of DCM. The resulting solution of acid chloride was added dropwise to a solution of 4-amino-benzoic acid methyl ester (4.30 g, 28.6 mmol; CAS 619-45-4), and TEA (8.0 mL, 57 mmol) in DCM (25 mL) while stirring at 0° C. under an argon atmosphere. The reaction mixture was warmed to room temperature over 18 hours and then quenched with water. The organic layer was separated, extracted sequentially with saturated aqueous NaHCO₃, 1M aqueous KHSO₄ and brine and then dried over anhydrous MgSO₄, filtered and concentrated in vacuo. The residue was dissolved in 10 mL THF and 10 mL water and treated with LiOH (1.20 g, 28.6 mmol) while stirring at room temperature. After 18 hours, the reaction mixture was quenched with 1M aqueous KHSO₄ and diluted with EtOAc. The organic layer was separated, extracted with water (3×), brine, dried (Na₂SO₄), filtered and concentrated in vacuo to yield an off-white solid. This material was triturated with hot EtOAc, cooled to room temperature and the resulting white precipitate was collected via filtration to yield the title Compound 7a (5.8g, 69%).

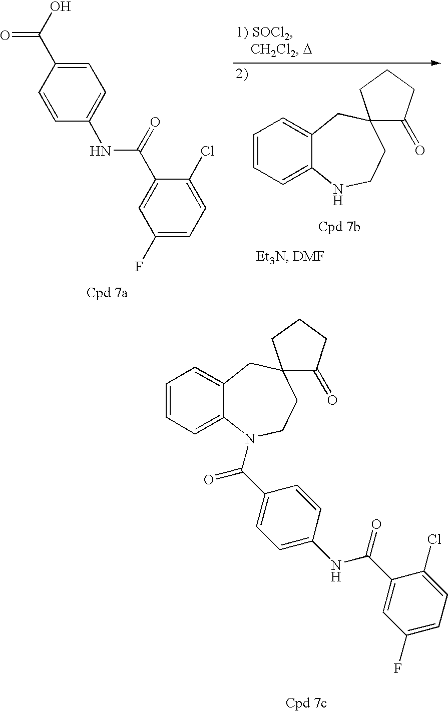

### Step B. 2-oxo-N-[4-(2-chloro-5-fluoro-phenylcarbonyl)amino-phenylcarbonyl]-spiro[cyclopentane-1,4′-benzo[b]azepane]

To a slurry of Compound 7a (1.61 g, 5.50 mmol) in DCM (15 mL) was added thionyl chloride (1.00 mL, 13.8 mmol) and refluxed under a drying tube. After 48 hours, the slurry was cooled to room temperature, concentrated in vacuo, dissolved in toluene, concentrated in vacuo and dissolved in 15 mL DCM. The resulting solution of acid chloride was added dropwise to a solution of 2-oxo-spiro[cyclopentane-1,4′-benzo[b]azepane] Compound 7b (1.08 g, 5.00 mmol; CAS 813426-37-8; US 2004/0259857 A1), TEA (1.5 mL, 11 mmol) and N,N-dimethylformamide (0.1 mL) in DCM (15 mL) while stirring at room temperature under an atmosphere of argon. After 18 hours, the reaction mixture was quenched with saturated aqueous NaHCO₃ and the organic layer was separated, extracted sequentially with saturated aqueous NaHCO₃ and brine, and then dried (Na₂SO₄), filtered and concentrated in vacuo. The residue was purified via column chromatography on silica gel eluting with EtOAc/hexane (1:1) to yield the title Compound 7c (1.69 g, 69%).

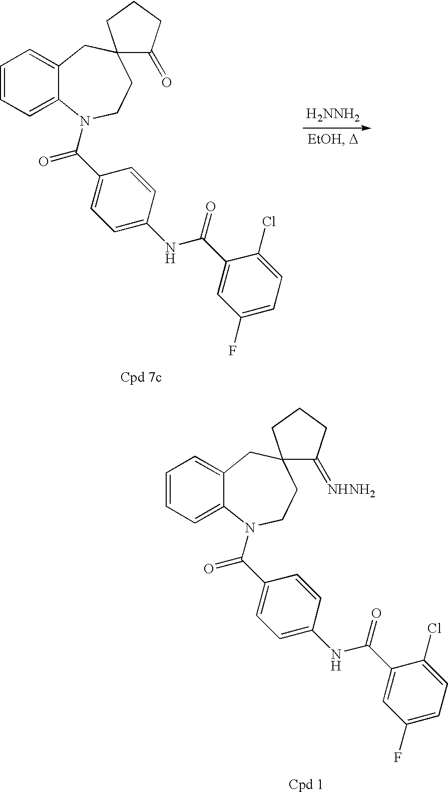

### Step C. 2-aminoimino-N-[4-(2-chloro-5-fluoro-phenylcarbonyl)amino-phenylcarbonyl]-spiro[cyclopentane-1,4′-benzo[b]azepane]

A solution of Compound 7c (115 mg, 0.23 mmol) and hydrazine (0.029 mL, 0.92 mmol) in absolute ethanol (5 mL) was refluxed for 18 hours. The reaction mixture was cooled to room temperature and concentrated in vacuo and the residue was purified via thin layer chromatography on silica gel eluting with methanol/DCM (3:1) to yield Compound 1 as a white solid (62.2 mg, 53%): ¹H NMR (CD₃OH) δ 7.6-6.9 (m, 10H), 6.7 (m, 1H), 1.20-3.0 (m, 12H); MS (ESI) m/z505(MH)⁺.

Using the procedure of Example 7, other compounds representative of the present invention were prepared:

| Cpd | Name and Data |
|---|---|
| 2 | N-[4-(2-chloro-5-fluoro-phenylcarbonyl)amino-phenylcarbonyl]-2- |
| | methoxyimino-spiro[cyclopentane-1,4′-benzo[b]azepane] |
| | ¹H NMR (CD₃OD) δ 7.50 (m, 3H), 7.1-7.3 (m, 6H), 7.02 (m, 1H), 6.71 (m, |
| | 1H), 3.85 (s, 3H), 2.45-3.0 (m, 4H), 2.23 (m, 1H), 1.55-1.9 (m, 6H), |
| | 1.44 (m, 1H); MS (ESI) m/z: 520 (MH)⁺. |
| 3 | N-[4-(2-chloro-5-fluoro-phenylcarbonyl)amino-phenylcarbonyl]-2- |
| | dimethylaminoimino-spiro[cyclopentane-1,4′-benzo[b]azepane] |
| | MS (ESI) m/z: 533 (MH)⁺. |
| 4 | N-[4-(2-chloro-5-fluoro-phenylcarbonyl)amino-phenylcarbonyl]-2-pyridin- |
| | 3-ylcarbonylaminoimino-spiro[cyclopentane-1,4′-benzo[b]azepane] |
| | ¹H NMR (CD₃OD) δ 9.00 (s, 1H), 8.73 (m, 1H), 8.28 (d, J = 7.7 Hz, 1H), |
| | 7.60-6.90 (m, 11H), 6.74 (d, J = 7.7 Hz, 1H), 3.76-0.9 (m, 12H); MS (ESI) |
| | m/z: 610 (MH)⁺. |
| 7 | (R)—N-[4-(2-chloro-5-fluoro-phenylcarbonyl)amino-phenylcarbonyl]-2- |
| | hydroxyimino-spiro[cyclopentane-1,4′-benzo[b]azepane] |
| | Compound 7 was prepared as a racemic mixture then the isomers were |
| | separated on a Chiralpak AD column eluted with ethanol/acetonitrile (4:1). |
| | [α]_{D}²³ = +227° (c = 1.00, CHCl₃); ¹H NMR (CDCl₃) δ 7.81 (s, 1H), 7.49 (m, |
| | 4H), 7.3-7.05 (m, 6H), 6.70 (d, J = 6.8 Hz, 1H), 4.99 (d, J = 12.7 Hz, 1H), |
| | 3.40 (m, 1H), 2.92-2.70 (m, 5H), 2.21 (m, 1H), 1.85 (m, 3H), 1.64 (m, 1H), |
| | 1.54 (m, 1H); MS (ESI) m/z: 506 (MH)⁺; Anal. |
| | C₂₈H₂₅N₃O₃ClF•1.4CF₃CO₂H•0.57H₂O: Calc'd: C, 54.73; H, 4.11; N, 6.22; |
| | Cl, 5.25; F, 14.62; H₂O, 1.52. Found: C, 54.27; H, 3.77; N, 6.04; F, 14.21; |
| | H₂O, 1.13. |
| 10 | N-[6-(2-chloro-5-fluoro-phenylcarbonyl)amino-pyridin-3-ylcarbonyl]-2- |
| | hydroxyimino-spiro[cyclopentane-1,4′-benzo[b]azepane] |
| | ¹H NMR (CD₃OD) δ 8.14 (s, 1H), 8.00 (d, J = 8.6 Hz, 1H), 7.69-7.60 (m, |
| | 2H), 7.37-7.02 (m, 5H), 6.82 (d, J = 7.6 Hz, 1H), 3.41 (d, J = 14.0 Hz, 1H), |
| | 3.03 (t, J = 12.7 Hz, 1H), 2.77 (d, J = 13.9 Hz, 1H), 2.62 (m, 2H), 2.27 (t, J = 11.9 Hz, |
| | 1H), 1.78 (m, 4H), 1.61 (m, 1H), 1.49 (m, 1H); MS (ESI) m/z: |
| | 507 (MH)⁺. |
| 37 | N-[4-(biphen-2-ylcarbonyl)amino-phenylcarbonyl]-2-hydroxyimino- |
| | spiro[cyclopentane-1,4′-benzo[b]azepane] |
| | ¹H NMR (CD₃OD) δ 7.57-6.99 (m, 16H), 6.66 (d, J = 7.5 Hz, 1H), 2.94 (m, |
| | 1H), 2.73-2.56 (m, 3H), 2.20 (m, 1H), 1.81-1.57 (m, 6H), 1.42 (m, 1H); MS |
| | (ESI) m/z: 530 (MH)⁺. |
| 38 | 2-aminoimino-N-[4-(biphen-2-ylcarbonyl)amino-phenylcarbonyl]- |
| | spiro[cyclopentane-1,4′-benzo[b]azepane] |
| | ¹H NMR (CD₃OD) δ 7.57-6.99 (m, 16H), 6.65 (d, J = 7.6 Hz, 1H), 3.41 (d, |
| | J = 14.0 Hz, 1H), 2.90 (t, J = 12.8 Hz, 1H), 2.63 (d, J = 13.9 Hz, 1H), |
| | 2.44 (m, 2H), 2.19 (t, J = 12.2 Hz, 1H), 1.91-1.29 (m, 6H); MS (ESI) m/z: |
| | 529 (MH)⁺. |
| 41 | N-[4-(3-fluoro-phenylcarbonyl)amino-phenylcarbonyl]-2-(2-amino- |
| | ethoxy)imino-spiro[cyclopentane-1,4′-benzo[b]azepane] |
| | ¹H NMR (CD₃OD) δ 7.72-7.00 (m, 11H), 6.73 (d, J = 7.7 Hz, 1H), 4.27 (m, |
| | 2H), 3.25-0.92 (m, 14H); MS (ESI) m/z: 515 (MH)⁺. |
| 42 | N-[3-methoxy-4-(2-chloro-5-fluoro-phenylcarbonyl)amino-phenylcarbonyl]- |
| | 2-hydroxyimino-spiro[cyclopentane-1,4′-benzo[b]azepane] |
| | ¹H NMR (DMSO-d6) δ 7.84 (d, J = 8.3 Hz, 1H), 7.6 (t, J = 6.8 Hz, 1H), |
| | 7.52 (d, J = 10.8, 1H), 7.32 (m, 2H), 7.14 (t, J = 7.2 Hz, 1H), 7.03 (t, J = 7.3 Hz, |
| | 1H), 6.76 (m, 3H), 4.74 (d, J = 12.8 Hz, 1H), 4.09 (bs, 1H), |
| | 3.57 (s, 3H), 3.16 (m, 3H), 2.87 (t, J = 12.7 Hz, 1H), 2.75 (d, J = 13.7 Hz, 1H), |
| | 2.07 (t, J = 11.6 Hz, 1H), 1.76-1.62 (m, 4H), 1.46 (m, 1H), 1.35 (m, 1H); |
| | MS (ESI) m/z: 536 (MH)⁺. |
| 43 | N-[4-(2-chloro-5-fluoro-phenylcarbonyl)amino-phenylcarbonyl]-2-pyridin- |
| | 2-ylaminoimino-spiro[cyclopentane-1,4′-benzo[b]azepane] |
| | ¹H NMR (CD₃OD) δ 8.02 (d, J = 3.75 Hz, 1H), 7.64-7.48 (m, 4H), |
| | 7.33-7.16 (m, 6H), 7.02 (m, 2H), 6.74 (m, 2H), 3.50-0.9 (m, 12H); MS (ESI) |
| | m/z: 582 (MH)⁺. |
| 44 | N-[4-(2-chloro-5-fluoro-phenylcarbonyl)amino-phenylcarbonyl]-2- |
| | hydroxyimino-spiro[cyclopentane-1,4′-benzo[b]azepane] |
| | ¹H NMR (CD₃OD) δ 7.53-6.99 (m, 10H), 6.71 (d, J = 7.6 Hz, 1H), 3.39 (d, |
| | J = 13.9 Hz, 1H), 2.97 (t, J = 13.5 Hz, 1H), 2.74 (d, J = 14 Hz, 1H), |
| | 2.62 (m, 2H), 2.24 (t, J = 11.8 Hz, 1H), 2.0-1.21 (m, 6H); MS (ESI) m/z: |
| | 506 (MH)⁺. |
| 45 | (S)—N-[4-(2-chloro-5-fluoro-phenylcarbonyl)amino-phenylcarbonyl]-2- |
| | hydroxyimino-spiro[cyclopentane-1,4′-benzo[b]azepane] |
| | Compound 46 was prepared as a racemic mixture then the isomers were |
| | separated on a Chiralpak AD column eluted with ethanol/acetonitrile (4:1). |
| | [α]_{D}²⁴ = −208.3° (c = 1.00, CHCl₃); ¹H NMR (CDCl₃) δ 8.36 (bm, 1H), |
| | 7.44-7.01 (m, 10H), 6.68 (d, J = 7.2 Hz, 1H), 4.96 (d, J = 12.8 Hz, 1H), 3.39 (d, |
| | J = 14 Hz, 1H), 2.95-2.69 (m, 5H), 2.21 (m, 1H), 1.93-1.49 (m, 6H); MS |
| | (ESI) m/z: 506 (MH)⁺. Anal. C₂₈H₂₅N₃O₃ClF•1•33CF₃CO₂H•0.25H₂O: |
| | Calc'd: C, 55.62; H, 4.08; N, 6.35; Cl, 5.35; F, 14.32; H₂O, 0.68. Found: |
| | C, 55.34; H, 3.77; N, 6.21; F, 14.10; H₂O, 0.67. |
| 46 | N-[4-pyrrolidin-1-yl-phenylcarbonyl]-2-hydroxyimino-spiro[cyclopentane- |
| | 1,4′-benzo[b]azepane] |
| | ¹H NMR (CD₃OD) δ 7.27 (d, J = 7.3 Hz, 1H), 7.06 (m, 4H), 6.68 (d, J = 7.4 Hz, |
| | 1H), 6.37 (d, J = 8.6 Hz, 2H), 3.36 (d, J = 13.9 Hz, 1H), 3.31 (m, 4H), |
| | 2.90 (t, J = 12.6 Hz, 1H), 2.71 (d, J = 13.5 Hz, 1H), 2.59 (m, 2H), 2.17 (d, |
| | J = 12.0 Hz, 1H), 1.98 (m, 4H), 1.82-1.71 (m, 5H), 1.59 (m, 1H); MS (ESI) |
| | m/z: 404 (MH)⁺. |
| 47 | N-[4-(3-methyl-1H-pyrazol-1-yl)-phenylcarbonyl]-2-hydroxyimino- |
| | spiro[cyclopentane-1,4′-benzo[b]azepane] |
| | ¹H NMR (CD₃OD) δ 8.06 (d, J = 2.2 Hz, 1H), 7.53 (d, J = 8.6 Hz, 2H), |
| | 7.30 (d, J = 8.8 Hz, 3H), 7.15 (t, J = 7.1 Hz, 1H), 7.00 (t, J = 7.6 Hz, 1H), |
| | 6.74 (d, J = 7.7 Hz, 1H), 6.30 (d, J = 2.3 Hz, 1H), 3.42 (d, J = 14.0 Hz, 1H), |
| | 3.00 (t, J = 12.6 Hz, 1H), 2.76 (d, J = 14.3 Hz, 1H), 2.64 (m, 2H), 2.31 (s, |
| | 3H), 1.76 (m, 4H), 1.61 (m, 1H), 1.47 (m, 1H); MS (ESI) m/z: 415 (MH)⁺. |
| 48 | N-[4-(2-methyl-5-fluoro-phenylcarbonyl)amino-phenylcarbonyl]-2- |
| | hydroxyimino-spiro[cyclopentane-1,4′-benzo[b]azepane] |
| | ¹H NMR (CD₃OD) δ 7.53 (d, J = 8.2 Hz, 2H), 7.29 (m, 2H), 7.19 (m, 5H), |
| | 7.03 (t, J = 7.4 Hz, 1H), 6.73 (d, J = 7.6 Hz, 1H), 3.41 (d, J = 14.0 Hz, |
| | 1H), 2.99 (t, J = 13.2 Hz, 1H), 2.75 (d, J = 14.2 Hz, 1H), 2.64 (m, 2H), |
| | 2.38 (s, 3H), 2.26 (t, J = 12.3 Hz, 1H), 1.78 (m, 4H), 1.72 (m, 1H), |
| | 1.62 (m, 1H); MS (ESI) m/z: 486 (MH)⁺. Anal. for |
| | C₂₉H₂₈N₃O₃F•1.3CF₃CO₂H•0.59H₂O: Calc'd: C, 58.90; H, 4.77; N, 6.52; |
| | F, 14.45; H₂O, 1.65. Found: C, 59.25; H, 4.37; N, 6.251; F, 14.53; H₂O, |
| | 2.04. |
| 49 | N-[4-(2-methyl-phenylcarbonyl)amino-phenylcarbonyl]-2-hydroxyimino- |
| | spiro[cyclopentane-1,4′-benzo[b]azepane] |
| | ¹H NMR (CD₃OD) δ 7.54 (d, J = 7.7 Hz, 2H), 7.40 (m, 2H), 7.29 (m, 3H), |
| | 7.18 (m, 3H), 7.03 (t, J = 7.3 Hz, 1H), 6.73 (d, J = 7.6 Hz, 1H), 3.41 (d, J = 13.9 Hz, |
| | 1H), 2.98 (t, J = 13.0 Hz, 1H), 2.75 (d, J = 14.2 Hz, 1H), 2.62 (m, |
| | 2H), 2.41 (s, 3H), 2.26 (t, J = 12.7 Hz, 1H), 1.77 (m, 4H), 1.62 (m, 1H), |
| | 1.47 (m, 1H); MS (ESI) m/z: 468 (MH)⁺. Anal. for C₂₉H₂₉N₃O₃F•0.59H₂O: |
| | Calc'd: C, 73.64; H, 6.33; N, 8.88; H₂O, 1.14. Found: C, 73.57; H, 6.45; N, |
| | 8.88; H₂O, 1.16. |
| 51 | N-[3-methoxy-4-(3-fluoro-phenylcarbonyl)amino-phenylcarbonyl]-2- |
| | hydroxyimino-spiro[cyclopentane-1,4′-benzo[b]azepane] |
| | ¹H NMR (CD₃OD) δ 7.97 (m, 3H), 7.24 (m, 4H), 7.04 (t, J = 7.5 Hz, 1H), |
| | 6.91 (t, J = 8.2 Hz, 1H), 6.84 (s, 1H), 6.76 (d, J = 7.6 Hz, 1H), 3.70 (s, |
| | 3H), 3.44 (d, J = 13.9 Hz, 1H), 3.01 (t, J = 13.2 Hz, 1H), 2.78 (d, J = 14.1 Hz, |
| | 1H), 2.65 (q, J = 8.3 Hz, 2H), 2.26 (t, J = 12.0 Hz, 1H), 1.79 (m, 4H), |
| | 1.62 (m, 1H), 1.48 (m, 1H); MS (ESI) m/z: 502 (MH)⁺. |

### Example 8

### N-[4-(2-chloro-5-fluoro-phenylcarbonyl)amino-phenylcarbonyl]-2-(1-methyl-1H-imidazol-2-yl)-spiro[cyclopent-2-ene-1,4′-benzo[b]azepane] (Cpd 5)

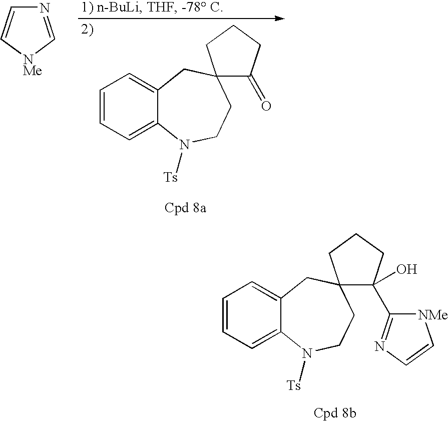

### Step A. 2-hydroxy-N-(4-methyl-phenylsulfonyl)-spiro[cyclopent-2-ene-1,4′-benzo[b]azepane]

To a solution of N-methyl-imidazole (0.215 mL, 2.7 mmol; CAS 616-47-7) in THF (3 mL) was added dropwise a solution of n-butyllithium in hexanes (2.5M, 0.860 mL, 2.2 mmol) while stirring at −78° C. under an atmosphere of argon. After 5 minutes, a solution of 2-oxo-N-(4-methyl-phenylsulfonyl)-spiro[cyclopentane-1,4′-benzo[b]azepane] Compound 8a (100 mg, 0.27 mmol; CAS 813426-36-7; US 2004/0259857 A1) in THF (3 mL) was added dropwise while stirring at −78° C. under an atmosphere of argon. The reaction mixture was allowed to warm to −55° C. over 45 minutes, quenched with saturated aqueous NH₄Cl, and allowed to warm to room temperature. The reaction mixture was diluted with EtOAc and the organic layer separated. The organic layer was extracted with water (2×), brine, dried (Na₂SO₄), filtered and concentrated in vacuo. The residue was purified via thin layer chromatography on silica gel eluting with methanol/DCM (1:49) to yield the title Compound 8b (92 mg, 75%).

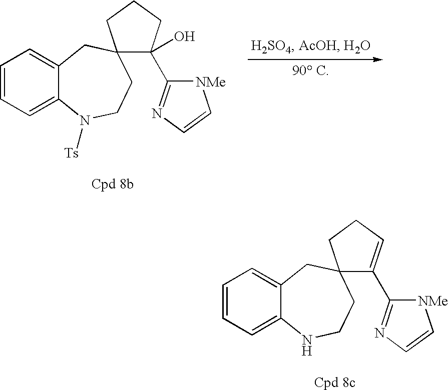

### Step B. 2-(1-methyl-1H-imidazol-2-yl)-spiro[cyclopent-2-ene-1,4′-benzo[b]azepane]

Compound 8b (92 mg, 0.20 mmol) was dissolved in sulfuric acid (1 mL), glacial acetic acid (1 mL), water (0.300 mL) and heated to 90° C. over 18 hours. The reaction mixture was cooled to room temperature then poured into 25 g of ice and diluted with EtOAc. The pH was adjusted to 7-8 using solid Na₂CO₃ and the organic layer was separated and extracted sequentially with saturated aqueous NaHCO₃ and brine, dried (Na₂SO₄), filtered and concentrated in vacuo to yield the title Compound 8c (43 mg, 77%).

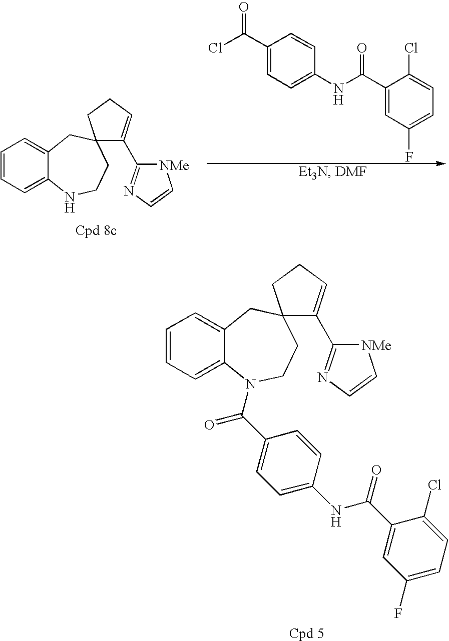

### Step C. N-[4-(2-chloro-5-fluoro-phenylcarbonyl)amino-phenylcarbonyl]-2-(1-methyl-1H-imidazol-2-yl)-spiro[cyclopent-2-ene-1,4′-benzo[b]azepane]

Using the procedure of Example 7, Step B, and substituting Compound 8c for Compound 7b, the product was purified via thin layer chromatography on silica gel eluting with methanol/DCM (5:95) to yield the title Compound 5 (5.6 mg 6%); ¹H NMR (CD₃OD) δ 7.81 (s, 1H), 7.49 (m, 3H), 7.30-7.05 (m, 6H), 6.99 (m, 2H), 6.66 (d, J=7.3 Hz, 1H), 6.06 (bs, 1H), 3.78 (bs, 1H), 3.71 (s, 3H), 2.93 (m, 1H), 2.81 (m, 1H), 2.56 (m, 2H), 2.31 (m, 1H), 1.86 (m, 1H), 1.69 (m, 2H), 1.28 (m, 1H); MS (ESI) m/z: 555 (MH)⁺.

### Example 9

### N-[4-(2-chloro-5-fluoro-phenylcarbonyl)amino-phenylcarbonyl]-2-dimethylamino-spiro[cyclopentane-1,4′-benzo[b]azepane] (Cpd 6)

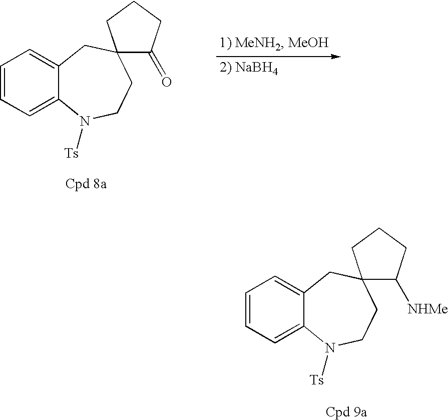

### Step A. 2-methylamino-N-(4-methyl-phenylsulfonyl)-spiro[cyclopentane-1,4′-benzo[b]azepane]

To a slurry of Compound 8a (1.00 g, 2.71 mmol; CAS 813426-36-7; US 2004/0259857 A1) in methanol (13 mL) was bubbled in methylamine gas while stirring at room temperature over 15 minutes. Sodium borohydride (307 mg, 8.13 mmol) was added and reaction mixture stirred at room temperature for 20 minutes and concentrated in vacuo. The residue was partitioned between EtOAc and water and the organic layer was separated, extracted with brine, dried (Na₂SO₄), filtered and concentrated in vacuo to yield the title Compound 9a (988 mg, 95%).

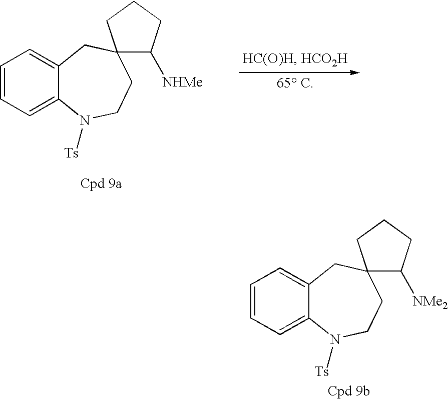

### Step B. 2-dimethylamino-N-(4-methyl-phenylsulfonyl)-spiro[cyclopentane-1,4′-benzo[b]azepane]

To a solution of Compound 9a (213 mg, 0.55 mmol) in methanol (2.5 mL) was added a 37% aqueous formaldehyde solution (2.5 mL) and formic acid (3 drops) while stirring at 65° C. After 1 hour, the reaction mixture was diluted with DCM (15 mL) and quenched with a solution of aqueous 0.1 M NaOH (20 mL, 2 mmol). The organic layer was separated, dried over anhydrous MgSO₄, filtered and concentrated in vacuo to afford the title Compound 9b (211 mg, 96%).

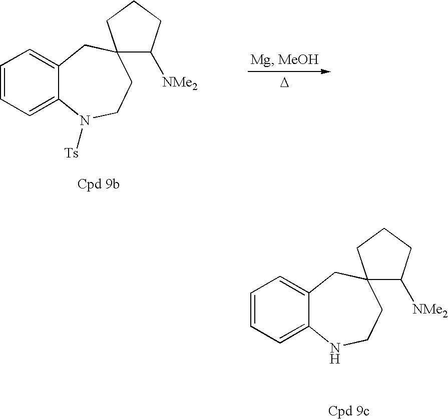

### Step C. 2-dimethylamino-spiro[cyclopentane-1,4′-benzo[b]azepane]

Compound 9b (211 mg, 0.53 mmol) was dissolved in anhydrous methanol (10 mL) and combined with magnesium turnings (257 mg, 10.6 mmol) and heated at reflux while stirring under an argon atmosphere over 5 hours. The reaction was cooled to room temperature, filtered through filter agent, and concentrated in vacuo. The residue was triturated 3 times with EtOAc and combined EtOAc triturations were filtered through filter agent and the filtrate was concentrated in vacuo to afford the title Compound 9c (108 mg, 83%).

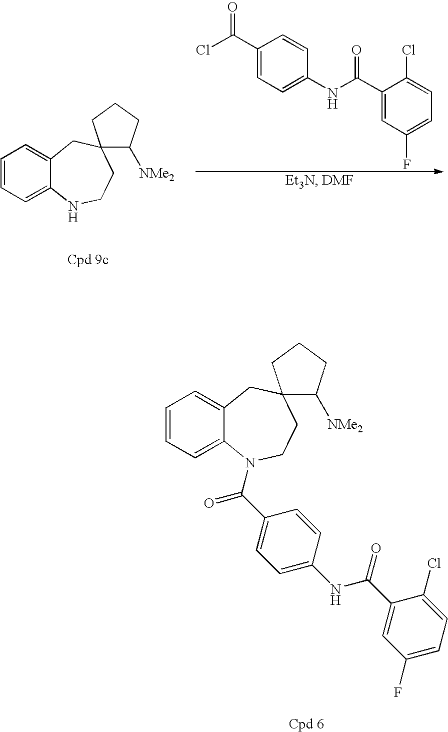

### Step D. N-[4-(2-chloro-5-fluoro-phenylcarbonyl)amino-phenylcarbonyl]-2-dimethylamino-spiro[cyclopentane-1,4′-benzo[b]azepane]

Using the procedure of Example 7, Step B, and substituting Compound 9c, the product was purified via reverse-phase column chromatography on a Cl 8 column eluting with acetonitrile/water with trifluoroacetic acid to yield the title Compound 6 (43 mg, 14%): ¹H NMR (CD₃OD) δ 8.23 (bs, 1H), 7.64-7.01 (m, 10H), 6.65 (d, J=7.5, 1H), 3.40-1.3 (m, 19H); MS (ESI) m/z: 520 (MH)⁺. Anal. for C₃₀H₃₁N₃O₂ClF.1.67CF₃CO₂H.0.6H₂O: Calc'd: C, 55.52; H, 4.73; N, 5.83; Cl, 4.92; F, 15.83; H₂O, 1.50. Found: C, 55.36; H, 4.52; N, 5.77; Cl, 4.85; F, 15.89; H₂O, 1.45.

Using the procedure of Example 9, other compounds representative of the present invention were prepared:

| Cpd | Name and Data |
|---|---|
| 33 | 2-aminosulfonylamino-N-[3-methoxy-4-(2-chloro-5-fluoro- |
| | phenylcarbonyl)amino-phenylcarbonyl]-spiro[cyclopentane-1,4′- |
| | benzo[b]azepane] |
| | MS (ESI) m/z: 601 (MH)⁺. |
| 39 | 2-amino-N-[4-(biphen-2-ylcarbonyl)amino-phenylcarbonyl]- |
| | spiro[cyclopentane-1,4′-benzo[b]azepane] |
| | ¹H NMR (CD₃OD) δ 7.57-6.99 (m, 16H), 6.65 (d, J = 7.6 Hz, 1H), |
| | 3.47-1.34 (m, 13H); MS (ESI) m/z: 516 (MH)⁺. |
| 50 | 2-amino-N-[3-methoxy-4-(3-fluoro-phenylcarbonyl)amino- |
| | phenylcarbonyl]-spiro[cyclopentane-1,4′-benzo[b]azepane] |
| | ¹H NMR (CD₃OD) δ 7.95 (m, 3H), 7.36 (t, J = 6.8 Hz, 1H), 7.24 (m, 3H), |
| | 7.07 (q, J = 6.8 Hz, 1H), 6.86 (m, 3H), 3.73 (s, 3H), 3.45 (d, J = 13.2 Hz, |
| | 1H), 3.17 (d, J = 13.9 Hz, 1H), 2.97 (q, J = 9.7 Hz, 1H), 2.73 (m, 1H), |
| | 2.43 (m, 1H), 2.34 (d, J = 9.3 Hz, 1H), 2.06 (t, J = 11.8 Hz, 1H), 1.83 (m, 4H), |
| | 1.54 (m, 1H), 1.39 (m, 1H); MS (ESI) m/z: 488 (MH)⁺. |

### Example 10

### (1R)-3-amino-N-[3-methoxy-4-(2-chloro-5-fluoro-phenylcarbonyl)amino-phenylcarbonyl]-spiro[cyclopentane-1,4′-benzo[b]azepane] (Cpd 17)

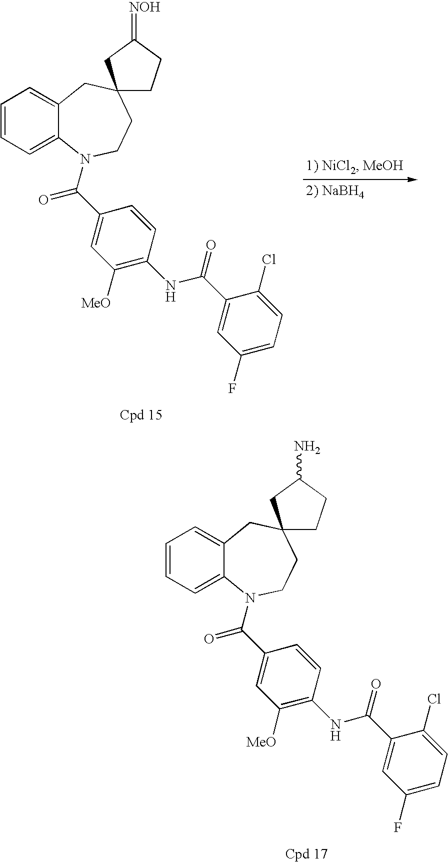

Compound 15 (650 mg, 1.2 mmol) and Nickel chloride (379 mg, 2.85 mmol) were combined in dry MeOH (28 mL) and cooled to −20° C. with a CCl₄/dry ice bath. Sodium borohydride (130 mg, 3.4 mmol) was added about every hour for 4 h while maintaining reaction temperature at −15 to −20° C. The reaction was stirred at ambient temperature for 1 h and then evaporated in vacuo to a white solid, which was partitioned between CHCl₃ and dilute NaOH. The organic layer was washed with water, dried (Na₂SO₄) and evaporated in vacuo to a white solid. A portion was purified by reverse phase HPLC (20-90% ACN) to afford Compound 17: ¹H NMR (CDCl₃) δ 8.64 (s, 1H), 8.25 (2d, 1H), 7.55-7.4 (m, 2H), 7.25-7.1 (m, 3H), 7.0-6.90 (m, 2H), 6.8-6.6 (m, 2H), 4.90-4.7 (m, 1H), 3.72/3.70 (2s, 3H), 3.65-2.9 (m, 5H), 2.8-2.6 (m, 1H), 2.2-1.8 (m, 3H), 1.8-1.4 (m, 5H/H2O); MS (ESI) m/z 522 (MH)⁺.

### Example 10a

### (1R)-3-aminosulfonylamino-N-[3-methoxy-4-(2-chloro-5-fluoro-phenylcarbonyl)amino-phenylcarbonyl]-spiro[cyclopentane-1,4′-benzo[b]azepane] (Cpd 19)

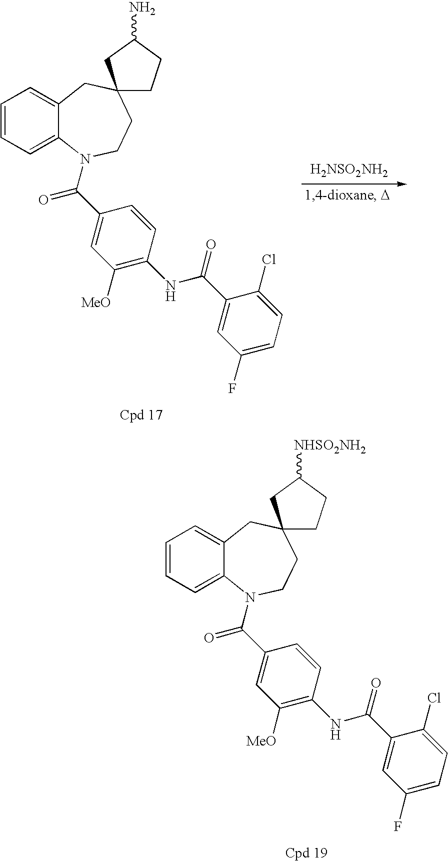

Compound 17 (200 mg, 0.38 mmol) and sulfamide (200 mg, 2.1 mmol) were combined in 1,4-dioxane (25 mL) and refluxed for 2 h. The reaction was cooled to room temperature and evaporated in vacuo to an oil, which was purified by flash column chromatography on silica gel (DCM/MeOH 20:1) and fractions were evaporated in vacuo to give Compound 19 as a white solid: ¹H NMR (CDCl₃) δ 8.65/8.59 (2s, 1.5H), 8.24 (d, J=8.5 Hz, 1H), 7.5-7.4 (m, 2H), 7.3-7.1 (m, 3H), 7.05-6.90 (m, 2H), 6.75-6.6 (m, 2H), 5.05 (m, 1H), 4.90-4.7 (m, 1H), 4.55 (s, 1H), 4.47 (s, 1H), 4.4-4.2 (m, 2H), 3.72 (s, 3H), 3.6-3.0 (m, 3H), 2.7-2.5 (m, 1H), 2.3-2.0 (m, 3H), 1.8-1.3 (m, 3H); MS (ESI) m/z 601 (MH)⁺.

Using the procedure of Example 10a, the diastereomeric compounds were separated by preparative thin layer chromatography on silica gel:

| Cpd | Name and Data |
|---|---|
| 34 | (1R,3S)-3-aminosulfonylamino-N-[3-methoxy-4-(2-chloro-5- |
| | fluoro-phenylcarbonyl)amino-phenylcarbonyl]-spiro[cyclopentane- |
| | 1,4′-benzo[b]azepane] |
| | MS (ESI) m/z: 601 (MH)⁺. |
| 35 | (1R,3R)-3-aminosulfonylamino-N-[3-methoxy-4-(2-chloro-5- |
| | fluoro-phenylcarbonyl)amino-phenylcarbonyl]-spiro[cyclopentane- |
| | 1,4′-benzo[b]azepane] |
| | MS (ESI) m/z: 601 (MH)⁺. |

### Example 11

### (1R)-N-[3-methoxy-4-(2-chloro-5-fluoro-phenylcarbonyl)amino-phenylcarbonyl]-3-(1H-pyrrolidin-1-yl)-spiro[cyclopentane-1,4′-benzo[b]azepane] (Cpd 25)

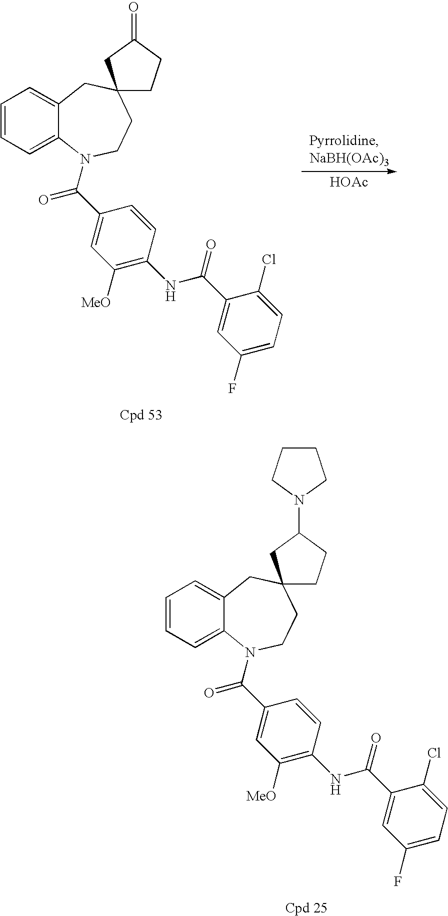

Compound 53 (100 mg, 0.19 mmol) and pyrrolidine (13.5 mg, 0.19 mmol) were combined in DCE (2 mL) followed by the addition of NaBH(OAc)₃ (57 mg, 0.27 mmol) and HOAC (11 mL, 0.19 mmol) and stirred at rt overnight. The reaction mixture was quenched with 1N NaOH (to pH 9), stirred for 5 min and the mixture was partitioned between water and chloroform. The organic layer was washed once with saturated NaHCO₃, once with brine, dried (Na₂SO₄), evaporated in vacuo to give crude product. The resulting oil was purifed by flash column chromatography on silica gel (DCM/MeOH/NH₄OH, 97:3:0.4) to give Compound 25 (69.4 mg) as a yellow gum: ¹HNMR (CDCl₃) δ 8.64 (s, 1H), 8.25 (t, 1H), 7.61-7.39 (m, 2H), 7.21-7.08 (m, 3H), 6.96-6.62 (m, 4H), 4.85 (m, 1H), 3.73-3.68 (m, 3H), 3.26-2.93 (m, 2H), 2.76-2.49 (m, 5H), 2.07-1.50 (m, 13 H); MS (ESI) m/z 576 (MH)⁺.

Using the procedure of Example 11, other compounds representative of the present invention were prepared:

| Cpd | Name and Data |
|---|---|
| 18 | (1R)—N-[3-methoxy-4-(2-chloro-5-fluoro-phenylcarbonyl)amino- |
| | phenylcarbonyl]-3-morpholin-4-yl-spiro[cyclopentane-1,4′-benzo[b]azepane] |
| | ¹HNMR (CDCl₃) δ 8.64 (s, 1H), 8.25 (m, 1H), 7.61-7.39 (m, 2H), |
| | 7.19-7.08 (m, 3H), 6.99-6.91 (m, 2H), 6.77-6.62 (m, 2H), 4.88 (m, 1H), 3.75-3.49 (m, |
| | 6H), 3.36-2.91 (m, 2H), 2.77-2.51 (m, 6H), 2.09-1.80 (m, 3H), 1.74-1.64 (m, |
| | 3H), 1.58-1.48 (m, 3H); MS (ESI) m/z: 592 (MH)⁺. |
| 20 | (1R)—N-[3-methoxy-4-(2-chloro-5-fluoro-phenylcarbonyl)amino- |
| | phenylcarbonyl]-3-dimethylamino-spiro[cyclopentane-1,4′-benzo[b]azepane] |
| | ¹HNMR (CDCl₃) δ 8.64 (s, 1H), 8.29-8.22 (m, 1H), 7.61-7.33 (m, 2H), |
| | 7.18-7.09 (m, 3H), 7.08-6.91 (m, 2H), 6.85-6.62 (m, 2H), 4.81 (m, 1H), 3.70 (d, |
| | 3H), 3.25-2.58 (m, 4H), 2.41-2.29 (m, 6H), 2.18-1.96 (m, 3H), 1.81-1.52 (m, |
| | 4H); MS (ESI) m/z: 550 (MH)⁺. |
| 21 | (1R)—N-[3-methoxy-4-(2-chloro-5-fluoro-phenylcarbonyl)amino- |
| | phenylcarbonyl]-3-(2-dimethylamino-ethyl)amino-spiro[cyclopentane-1,4′- |
| | benzo[b]azepane] |
| | ¹HNMR (CDCl₃) δ 8.63 (s, 1H), 8.25 (m, 1H), 7.61-7.39 (m, 2H), |
| | 7.23-7.10 (m, 3H), 7.03-6.91 (m, 2H), 6.88-6.61 (m, 2H), 4.82 (m, 1H), 3.70 (d, 3H), |
| | 3.29-2.92 (m, 4H), 2.75-2.61 (m, 4H), 2.57-2.34 (m, 2H), 2.24-2.20 (m, 6H), |
| | 2.13-1.90 (m, 3H), 1.84-1.78 (m, 1H), 1.76-1.45 (m, 3H); MS (ESI) m/z: |
| | 593 (MH)⁺. |
| 23 | (1R)—N-[3-methoxy-4-(2-chloro-5-fluoro-phenylcarbonyl)amino- |
| | phenylcarbonyl]-3-(4-methyl-piperazin-1-yl)-spiro[cyclopentane-1,4′- |
| | benzo[b]azepane] |
| | ¹HNMR (CDCl₃) δ 8.63 (s, 1H), 8.25 (d, J = 8.2 Hz, 1H), 7.61-7.38 (m, 2H), |
| | 7.21-7.10 (m, 3H), 7.08-6.90 (m, 2H), 6.77-6.61 (m, 2H), 4.83 (m, 1H), |
| | 3.70 (d, 3H), 3.26-2.92 (m, 4H), 2.71-2.46 (m, 6H), 2.29 (d, 4H), 2.17-1.89 (m, |
| | 3H), 1.79-1.48 (m, 6H); MS (ESI) m/z: 605 (MH)⁺. |

### Example 12

### 2-(aminomethylcarbonyl)amino-N-[4-(biphen-2-ylcarbonyl)amino-phenylcarbonyl]-spiro[cyclopentane-1,4′-benzo[b]azepane] (Cpd 40)

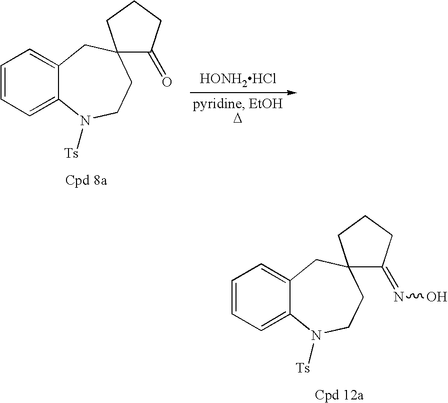

### Step A N-(4-methyl-phenylsulfonyl)-2-hydroxyimino-spiro[cyclopentane-1,4′-benzo[b]azepane]

A slurry of Compound 8a (1.19 g, 3.22 mmol; CAS 813426-36-7; US 2004/0259857 A1), hydroxylamine hydrochloride (895 mg, 12.9 mmol) and pyridine (8 mL) in ethanol (43 mL) was refluxed under a drying tube for 18 hours. The reaction mixture was cooled to room temperature and concentrated in vacuo. The residue was dissolved in EtOAc, extracted with water (3×) and brine, then dried (Na₂SO₄), filtered and concentrated in vacuo to yield the title Compound 12a (1.5 g, 100%).

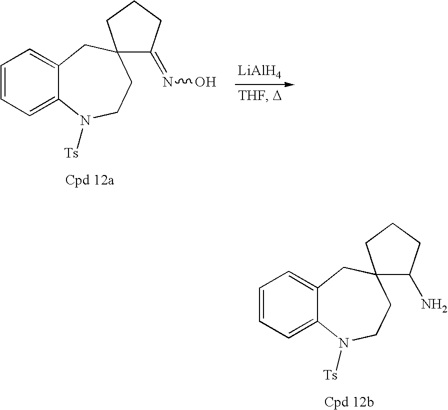

### Step B. 2-amino-N-(4-methyl-phenylsulfonyl)-spiro[cyclopentane-1,4′-benzo[b]azepane]

To a solution of Compound 12a (1.24 g, 3.22 mmol) in anhydrous THF (25 mL) was added a solution of lithium aluminum hydride in THF (1 M, 12.9 mL, 12.9 mmol) and refluxed 1 hour under an atmosphere of argon. The reaction mixture was cooled on an ice bath and quenched with a solution of 3M aqueous NaOH. The resulting slurry was filtered through filter agent and the filtrate concentrated in vacuo. The residue was dissolved in EtOAc, extracted with water (2×) and brine, then dried (Na₂SO₄), filtered and concentrated in vacuo to yield the title Compound 12b (943 mg, 79%).

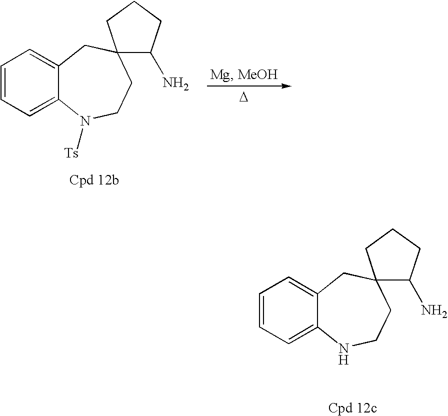

### Step C. 2-amino-spiro[cyclopentane-1,4′-benzo[b]azepane]

Using the procedure of Example 9, Step C above, Compound 12b was carried forward to yield the title Compound 12c (549 mg, 100%).

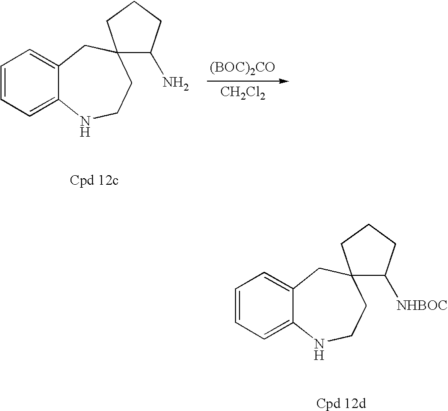

### Step D. 2-tert-butoxycarbonylamino-spiro[cyclopentane-1,4′-benzo[b]azepane]

To a solution of Compound 12c (549 mg, 2.54 mmol) in DCM (20 mL) was added di-tert-butyl dicarbonate (554 mg, 2.54 mmol) while stirring at room temperature. After 4 hours, the reaction mixture was concentrated in vacuo and the residue was purified via column chromatography on silica gel eluting with EtOAc/hexane (1:4) to yield the title Compound 12d (209 mg, 26%).

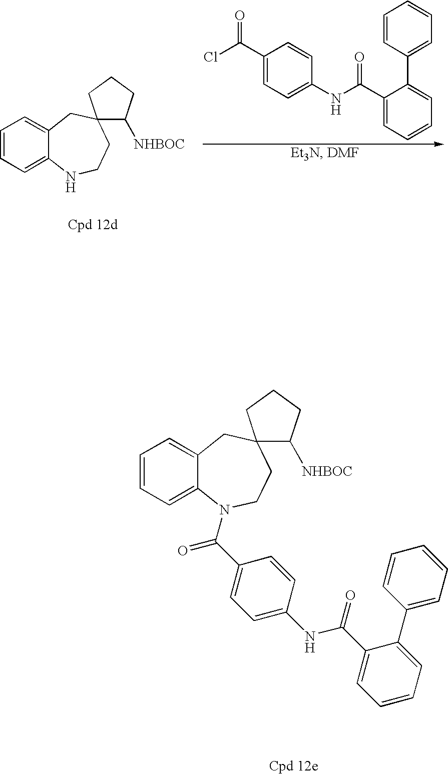

### Step E. 2- tert-butoxycarbonylamino-N-[4-(biphen-2-ylcarbonyl)amino-phenylcarbonyl]-spiro[cyclopentane-1,4′-benzo[b]azepane]

Using the procedure of Example 7, Step B above, Compound 12d was reacted with acid chloride formed from 4-[(biphenyl-2-carbonyl)-amino]-benzoic acid (CAS 16826-74-2; US 2004/0259857 A1). The crude product was purified via column chromatography on silica gel eluting with EtOAc/hexanes (7:13) to yield the title Compound 12e (332 mg, 85%).

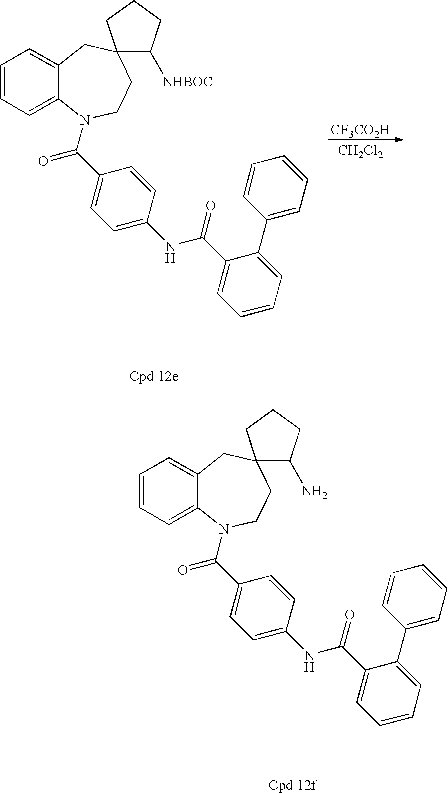

### Step F 2-amino-N-[4-(biphen-2-ylcarbonyl)amino-phenylcarbonyl]-spiro[cyclopentane-1,4′-benzo[b]azepane]

To a solution of Compound 12e (332 mg, 0.54 mmol) in DCM (10 mL) was added trifluoroacetic acid (10 mL) while stirring at room temperature for 20 minutes. The reaction mixture was concentrated in vacuo. The residue was dissolved in EtOAc, extracted with saturated aqueous NaHCO₃ and brine, then dried (Na₂SO₄), filtered and concentrated in vacuo. The residue was purified via preparative thin layer chromatography on silica gel eluting with DCM/MeOH (9:1) to yield the title Compound 12f (33 mg, 12%).

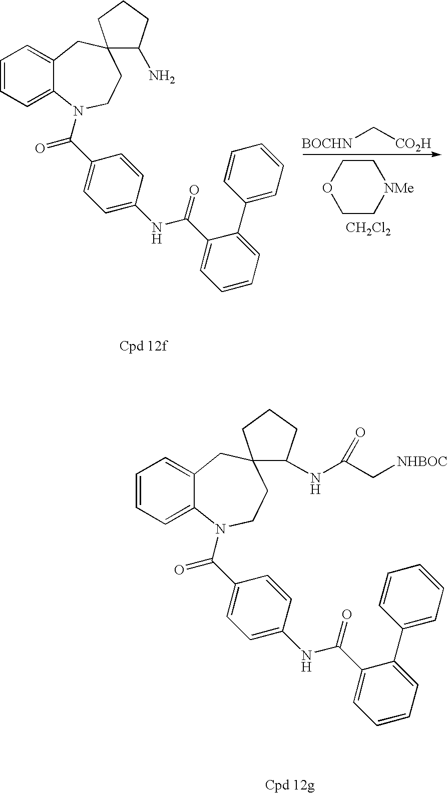

### Step G 2-[(tert-butoxycarbonyl)aminomethylcarbonyl]amino-N-[4-(biphen-2-ylcarbonyl)amino-phenylcarbonyl]-spiro[cyclopentane-1,4′-benzo[b]azepane]

To a solution of tert-butoxycarbonylamino-acetic acid (12 mg, 0.07 mmol) and 4-methyl-morpholine (0.015 mL, 0.14 mmol) in THF (1 mL) was added isopropyl chloroformate (0.009 mL, 0.07 mmol) while stirring at room temperature under an argon atmosphere for 30 minutes. A solution of Compound 12f (33 mg, 0.64 mmol) in THF (1 mL) was added. After 30 minutes, the reaction mixture was quenched with water and diluted with EtOAc. The organic layer was separated, extracted with water, 10% aqueous citric acid, saturated aqueous NaHCO₃ and brine, then dried (Na₂SO₄), filtered and concentrated in vacuo. The residue was purified via preparative thin layer chromatography on silica gel eluting with DCM/MeOH (19:1) to yield the title Compound 12g (9 mg, 21%).

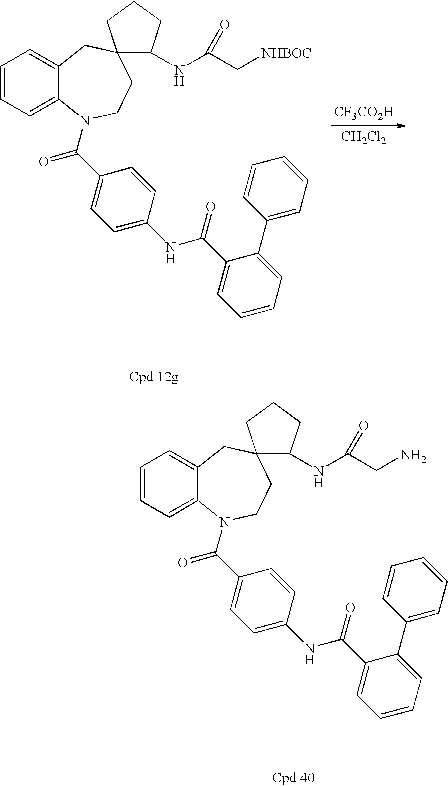

### Step H 2-(aminomethylcarbonyl)amino-N-[4-(biphen-2-ylcarbonyl)amino-phenylcarbonyl]-spiro[cyclopentane-1,4′-benzo[b]azepane]

Compound 12g (9 mg, 0.013 mmol) was dissolved in a solution of TFA (5 mL) in DCM (5 mL) and stirred at room temperature for 30 minutes. The reaction mixture was concentrated in vacuo and the residue was triturated twice with diethyl ether. The resulting solid was purified by preparative thin layer chromatography on silica gel eluting with DCM/MeOH (9:1) to provide the title Compound 40 (1.2 mg, 16%) as a white solid: ¹H NMR (CD₃OD) δ 7.57-6.99 (m, 17H), 3.47-0.9 (m, 15H); MS (ESI) m/z: 573 (MH)⁺.

### Example 13

### 2-amino-N-[3-methoxy-4-(2-chloro-5-fluoro-phenylcarbonyl)amino-phenylcarbonyl]-spiro[cyclopentane-1,4′-benzo[b]azepane](Cpd 8)

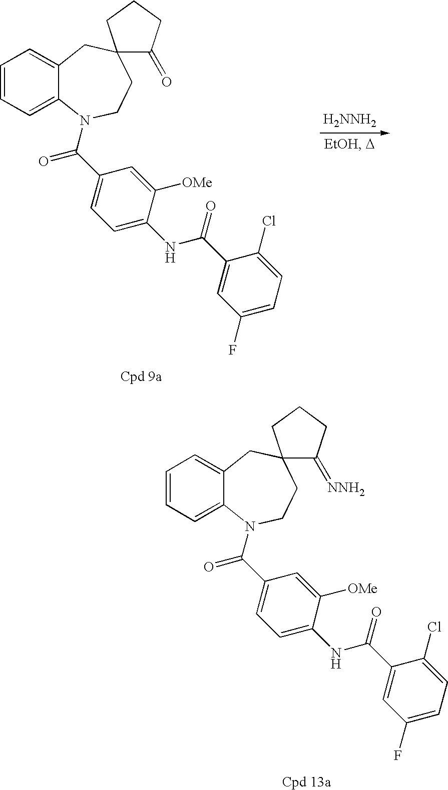

### Step A 2-aminoimino-N-[4-(2-chloro-5-fluoro-phenylcarbonyl)amino-(3-methoxy-phenylcarbonyl)]-spiro[cyclopentane-1,4′-benzo[b]azepane]

Using the procedure of Example 7, Step C, N-[4-(2-chloro-5-fluoro-phenylcarbonyl)amino-phenylcarbonyl]-2-oxo-spiro[cyclopentane-1,4′-benzo[b]azepane] Compound 9a (130 mg, 0.25 mmol; CAS 813426-39-0; US 2004/0259857 A1) was converted to the title Compound 13a (134 mg).

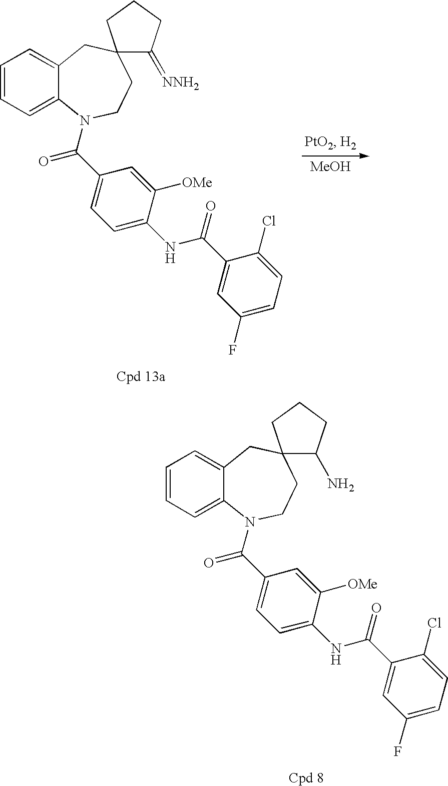

### Step B. 2-amino-N-[3-methoxy-4-(2-chloro-5-fluoro-phenylcarbonyl)amino-phenylcarbonyl]-spiro[cyclopentane-1,4′-benzo[b]azepane]

Compound 13a (134 mg, 0.25 mmol) was hydrogenated on a Parr apparatus (50 psig H₂) with platinium (IV) oxide (15 mg) in methanol (5 mL) at room temperature. After 16 hours, the reaction mixture was filtered through filter agent and concentrated in vacuo. The residue was purified on reverse-phase HPLC C18 column eluting with acetonitrile/water/trifluoroacetic acid to give the title Compound 8 (74 mg, 41%) as a white powder: ¹H NMR (CD₃OD) δ 8.05-7.92 (m, 2H), 7.64 (m, 1H), 7.38-7.07 (m, 4H), 6.87-6.76 (m, 4H), 3.71 (s, 3H), 3.47-1.34 (m, 13H); MS (ESI) m/z 522 (MH)⁺.

Using the procedure of Example 13, other compounds representative of the present invention were prepared:

| Cpd | Name and Data |
|---|---|
| 9 | N-[3-methoxy-4-(2-chloro-5-fluoro-phenylcarbonyl)amino- |
| | phenylcarbonyl]-2-hydroxyimino-spiro[cyclopentane-1,4′- |
| | benzo[b]azepane] |
| | ¹H NMR (CD₃OD) δ 8.03 (m, 1H), 7.64 (m, 1H), |
| | 7.36-7.06 (m, 5H), 6.87-6.77 (m, 3H), 3.71 (s, 3H), |
| | 3.47-1.34 (m, 13H); MS (ESI) m/z 538 (MH)⁺. |

### BIOLOGICAL EXAMPLES

The ability of the compounds for treating a vasopressin receptor mediated condition was determined using the following procedures.

### Example 1

### In-Vitro Binding Assay

Assay buffer is 50 mM Tris-Cl, 5 mM MgCl₂, 0.1% BSA (pH 7.5) containing 5 μg/mL of aprotinin, leupeptin, pepstatin, 50 μg/mL bacitracin, and 1 mM Pefabloc (4-(2-aminoethyl)-benzenesulfonyl fluoride, hydrochloride manufactured by Roche Diagnostics Corporation, Indianapolis, Ind. and distributed by Boehringer Mannheim). H3 vasopressin is ³H-arginine-8-vasopressin (NEN Life Sciences, Boston, Mass.; 68.5 Ci/mmol, final concentration in assay is 0.65-0.75 nM). Into the wells of a 96-well round bottom polypropylene plates are added buffer, test compound, membrane (containing human V1a or V2 receptor), and H3 vasopressin. The reaction plates are allowed to sit at room temperature for one hour. The samples are filtered through Unifilter GF/C plates (PerkinElmer Life Sciences, Boston, Mass.) presoaked in 0.3 polyethyleneimine. The plates are washed 5 times with cold physiological saline containing 0.05% Tween 20. After drying, the bottom of the filter plates are sealed and 0.025 mL of Microscint-20 (Packard Instrument Co, Meriden, Conn.) is added to each filter. The top of the plate is sealed, and the plate is counted. Non-specific binding is determined by the addition of 1.25 μM arginine-8-vasopressin in those wells.

The % inhibition for the test results was calculated according to the formula:

$$\% Inhibition = 100 - 100 \times \frac{peak response after drug}{peak response before drug}$$

The results for compounds tested are shown in Table 1.

Where an IC₅₀ value was not obtained, the percent inhibition values are shown in parentheses (*) and were obtained at a test concentration of 0.2 μM.

TABLE 1
V1a and V2 (binding) IC₅₀ (μM)

| Cpd | V1a IC₅₀ | V2 IC₅₀ |
|---|---|---|
| 1 | 0.003 | 0.023 |
| 2 | 0.007 | 0.044 |
| 3 | 0.009 | (48%) |
| 4 | 0.012 | 0.062 |
| 5 | 0.009 | 0.084 |
| 6 | 0.010 | (63%) |
| 7 | 0.008 | 0.028 |
| 8 | 0.016 | 0.083 |
| 9 | 0.017 | 0.047 |
| 10 | 0.060 | 0.070 |
| 37 | 0.023 | 0.027 |
| 38 | 0.021 | 0.024 |
| 39 | 0.080 | 0.11 |
| 40 | 0.023 | 0.015 |
| 41 | 0.008 | (55%) |
| 42 | 0.012 | 0.039 |
| 43 | 0.005 | 0.056 |
| 44 | 0.007 | 0.028 |
| 45 | (67%) | (10%) |
| 46 | (73%) | (40%) |
| 47 | (86%) | (62%) |
| 48 | 0.016 | 0.026 |
| 49 | 0.012 | 0.034 |
| 50 | (89%) | (9%) |
| 51 | 0.016 | 0.087 |

### Example 2

### V1a Vasopressin Receptor Functional Activity

The V1a receptor is a G-protein coupled receptor, which upon activation triggers an increase in intracellular calcium mobilization. To evaluate compounds for their functional V1a receptor activity, HEK-293 cells were transfected with the human V1a receptor (V1a-HEK cells). HEK-293 cells were grown in DMEM (Dulbecco's modified Eagle Media) supplemented with 10% FBS and glutamine. HEK-cells were passed biweekly by trypsinization and seeded into 96 well plates at 33,000 cells per well. HEK-293 cells were transfected with human V1a receptor DNA using DMRIE-C reagent from Life Technologies (Carlsbad, Calif.). Stable lines were generated by selecting cells grown in culture media containing geneticin. After growing in Packard Clear-View black 96 well plates for 4-6 days, V1a-HEK cells were loaded with the calcium-sensitive fluorescence dye, FLUO-3 AM. Changes in intracellular calcium mobilization were measured by quantitating intracellular fluorescence using FLIPR (Fluorometric Imaging Plate Reader; Molecular Devices, Sunnyvale, Calif.). Test compounds were first added to the cells and the resulting changes in fluorescence measured to detect receptor agonistic activity. Five minutes later the cells were challenged with vasopressin to test compounds for their antagonistic activity. Receptor antagonists inhibit the ability of vasopressin to stimulate increases in intracellular fluorescence.

The results for compounds tested are shown in Table 2.

TABLE 2
V1 (functional) IC₅₀ (μM)

| Cpd | IC₅₀ |
|---|---|
| 1 | 0.010 |
| 3 | 0.020 |
| 4 | 0.020 |
| 5 | 0.130 |
| 6 | 0.070 |
| 7 | 0.018 |
| 9 | 0.052 |
| 11 | 0.063 |
| 12 | 0.32 |
| 13 | 0.027 |
| 14 | 0.095 |
| 15 | 0.090 |
| 16 | 0.030 |
| 17 | 0.73 |
| 18 | 0.24 |
| 19 | 0.10 |
| 20 | 0.49 |
| 21 | 0.17 |
| 22 | 0.29 |
| 23 | 0.71 |
| 24 | 0.10 |
| 25 | 0.81 |
| 26 | 4.0 |
| 27 | 0.13 |
| 28 | 0.22 |
| 29 | 0.17 |
| 30 | 0.21 |
| 31 | 0.15 |
| 32 | 0.17 |
| 33 | 0.33 |
| 34 | 0.25 |
| 35 | 0.078 |
| 36 | 0.21 |
| 37 | 0.030 |
| 38 | <0.030 |
| 39 | 0.73 |
| 40 | 0.29 |
| 41 | 0.020 |
| 42 | 0.060 |
| 43 | 0.080 |
| 44 | 0.020 |
| 45 | 0.57 |
| 52 | 0.077 |

### Example 3

### V2 Vasopressin Receptor Functional Activity

The V2 receptor is also a G-protein coupled receptor which when activated induces an increase in cAMP turnover. Antagonism against the V2 receptor is determined by measuring cAMP accumulation in transfected HEK-293 cells expressing the human V-2 receptor (V2-HEK cells). Compounds are tested for their ability to block the stimulatory effects of vasopressin on cAMP accumulation. The cell content of cAMP is measured by radioimmunoassay using NEN flashplates.

The results for compounds tested are shown in Table 3.

TABLE 3
V2 (functional) IC₅₀ (μM)

| Cpd | IC₅₀ |
|---|---|
| 1 | 0.054 |
| 3 | 0.25 |
| 4 | 0.043 |
| 5 | 0.066 |
| 6 | 0.107 |
| 7 | 0.024 |
| 11 | 0.040 |
| 12 | 1.1 |
| 13 | 0.011 |
| 14 | 0.73 |
| 15 | 0.40 |
| 16 | 0.074 |
| 17 | 1.1 |
| 18 | 1.2 |
| 19 | 0.12 |
| 21 | 1.1 |
| 22 | 0.74 |
| 24 | 0.22 |
| 25 | 1.0 |
| 26 | 0.070 |
| 27 | 0.39 |
| 28 | 0.20 |
| 29 | 0.090 |
| 30 | 0.090 |
| 31 | 0.14 |
| 32 | 0.034 |
| 33 | 0.031 |
| 34 | 0.25 |
| 35 | 0.044 |
| 36 | 0.17 |
| 37 | 0.090 |
| 38 | 0.040 |
| 39 | 0.69 |
| 40 | 0.20 |
| 41 | 0.70 |
| 42 | 0.092 |
| 43 | 0.20 |
| 44 | 0.066 |
| 45 | 0.55 |
| 52 | 0.035 |

### Example 4

### Reversal of Vasopressin-Induced Hypertension in Rats

The anti-hypertensive activity of a compound may be assessed using an anesthetized model of vasopressin-induced hypertension. Male Long Evans, normotensive rats of between 350 and 450 g in body weight are anesthetized with pentobarbital (35 mg/kg, ip) and maintained throughout the procedure with an ip infusion of 10 mg/kg/hr. Arginine vasopressin (AVP) is infused at 30 ng/kg/min, iv, to induce a stable hypertensive state (ca. 50 mm Hg increase in mean arterial blood pressure). Compounds of interest are administered in an ascending dose fashion and the maximum decrease in mean arterial blood pressure is recorded. An ED₅₀ is determined from the linear portion of the dose-response relationship for each animal.

### Example 5

### Diabetic Nephropathy Animal Models

Several animal models are believed to mimic various components of diabetic nephropathy in humans, in particular, the streptozotocin-induced model of type 1 diabetes in rats, the db/db genetic mouse model of type 2 diabetes and the 5/6 nephrectomy model of renal failure in rats. Compounds may be evaluated in the streptozotocin diabetic model by administering the compound at 1, 3 or 10 mg/kg/day for 12 weeks and monitored at several endpoints during the study that are indicative of diabetic kidney disease, including reduced urine albumin, serum creatinine levels and levels of various cytokines in urine. At the end of the study, morphologic changes in the kidney are evaluated histologically for comparison to normal kidneys. Similar studies are performed in the other two models to confirm activity.

### Example 6

### AVP Receptor Antagonists

Argenine-vasopressin (AVP) levels are dramatically elevated following ischemic stroke and head injury and contribute to the tissue inflammatory response. AVP receptor antagonists have been shown to block development of cerebral edema following traumatic brain injury and ischemic stroke by regulating water and electrolyte transport across the cerebrovascular endothelium (via endothelial V1a receptor inhibition) and by promoting diuresis (via renal V2 receptors). Additional neuroprotective actions of AVP receptor antagonists may be mediated by inhibition of neuronal V1a receptors. Thus, compounds of this invention may be useful in ischemic stroke and traumatic brain injury. V1a/V2 antagonists may reduce the post-ischemia inflammatory response and reduce the volume of brain tissue infarction following ischemic stroke. As many of the neuroprotective and anti-edema actions of AVP receptor antagonists are mediated at the level of the cerebrovascular endothelium or kidney, it is not essential that compounds cross the blood brain barrier. However, as noted above, CNS penetration may add benefit by limiting actions of AVP at neuronal V1a receptors.

### Example 7

### Rodent Model of Embolic Stroke

The pharmacokinetic properties of a compound may be determined in order to optimize plasma half-life and optimal dosing regimen. This includes evaluation the ability of these compounds to cross the blood-brain barrier, and direct measurement of drug concentrations and half-life in brain tissue. The neuroprotective and anti-edema properties of these compounds can be determined with a rodent model of embolic stroke.

In this model, an aliquot of the animal's blood is removed and refrigerated overnight to allow a thrombin-rich clot to form. This clot is then placed surgically at the origin of the middle cerebral artery and left in place for 2-4 hrs to produce prolonged cerebral ischemia. At this point the clot may be left in place permanently or the clot may be lysed using intravenous administration of recombinant tissue plasminogen activator (rt-PA) to allow reperfusion. The vasopressin receptor antagonists of this invention may be administered intravenously at various times following clot placement and may be given as a bolus dose, a bolus dose followed by continuous intravenous infusion or continuous intravenous infusion alone. Compound may be given at times ranging from two hours to one week following onset of ischemia to define the optimal treatment window. The acute intravenous dosing may also be followed by oral administration of the compound to determine the optimal treatment duration.

### Example 8

### Rodent Model of Traumatic Brain Injury

The vasopressin receptor antagonists of this invention may be profiled in a rodent model of traumatic brain injury. This model requires opening a cranial window to expose the dura matter. A controlled, measured weight is then dropped on the dura to induce injury. This model is well characterized and produces a defined pattern of neuronal cell loss and inflammation.

Edema, inflammation and neuroprotection may be determined using one or more of the following approaches: Animals may be euthanized at various time points following ischemia, from 24 hrs to four weeks, and the volume of infarction and brain edema may be measured using standard histological and histochemical methods. Animals may also be subjected to MRI imaging so that the evolution of infarction and edema can be measured within the same animal. Finally, histological and histochemical measurements of blood-brain barrier integrity and infiltration of inflammatory cells (e.g., monocytes, macrophages, microglial cells) may be performed and used for quantitative analyses.

Finally, all animals may be evaluated in a comprehensive series of behavioral assays to evaluate the effects of vasopressin receptor antagonists on neurological function and behavior. These behavioral assessments may include a global neurological assessment, evaluation of motor asymmetry and assessment of sensorimotor integration using assays such as the foot-fault, Rotarod and beam-balance tests.

While the foregoing specification teaches the principles of the present invention, with examples provided for the purpose of illustration, it will be understood that the practice of the invention encompasses all of the usual variations, adaptations and/or modifications as come within the scope of the following claims and their equivalents.

What is claimed is:

1. A compound having the general structure shown in Formula (I): 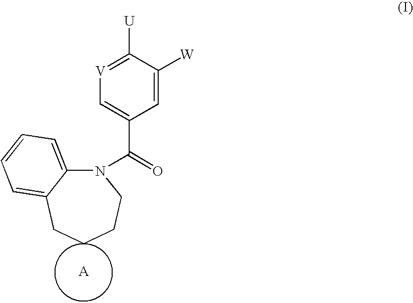
or a form thereof, wherein,
Ring A is selected from the group consisting Ring R₁a, Ring R₁b, Ring R₁c, Ring R₁d, Ring R₁e, Ring R₁f, Ring R₁g, Ring R₁h, Ring R₁i, Ring R₂a, Ring R₂b, Ring R₂c, Ring R₂d, Ring R₂e, Ring R₂f, Ring R₂g, Ring R₂h, and Ring R₂i, of the formulae: 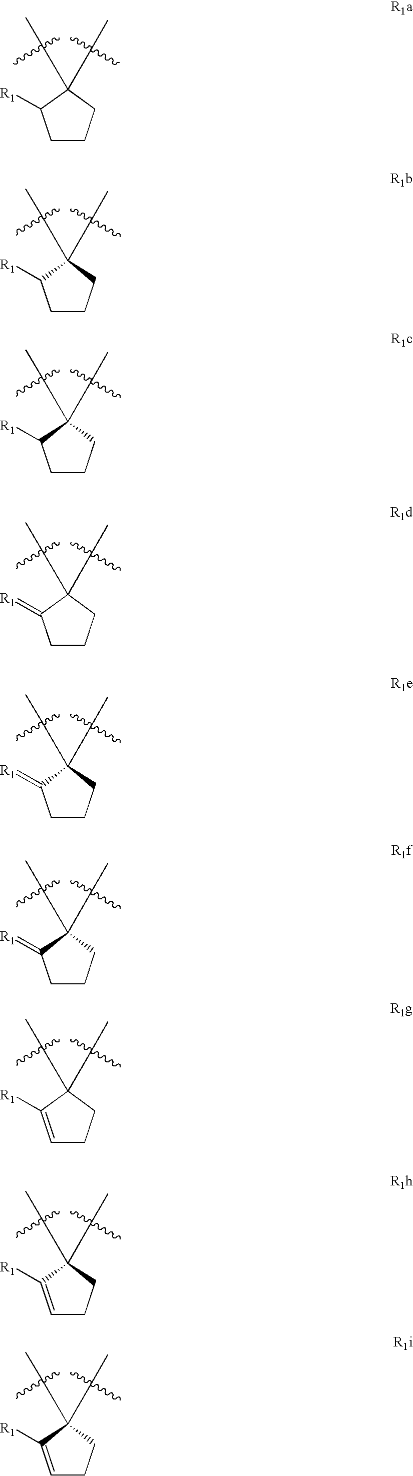 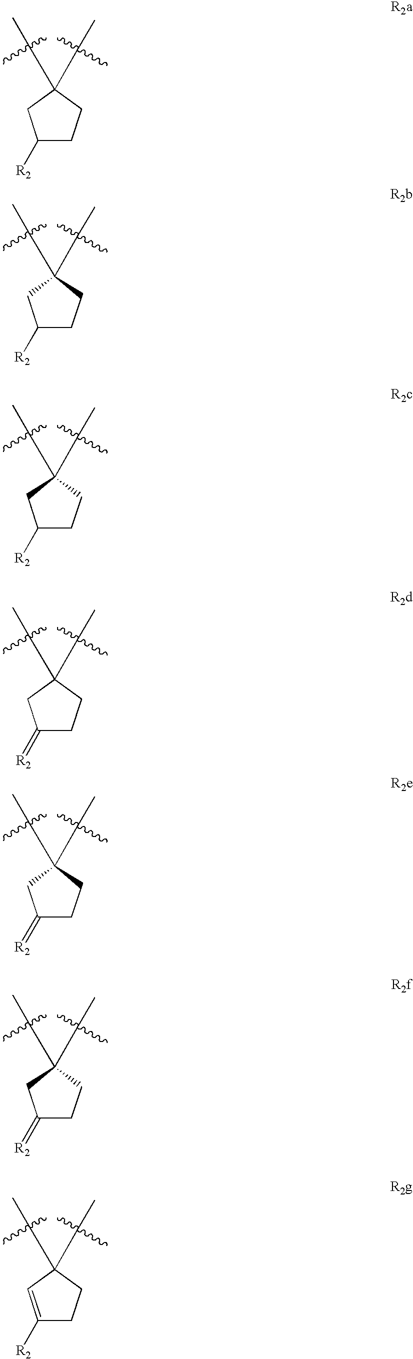 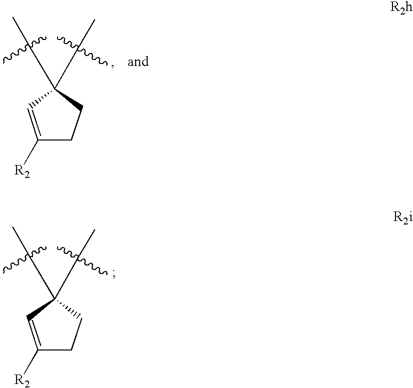
U is phenyl-carbonyl-amino, biphenyl-carbonyl-amino, heterocyclyl or heteroaryl,
wherein heterocyclyl and heteroaryl are each optionally substituted with C₁₋₄alkyl, and
wherein each phenyl is optionally substituted with one, two or three substituents independently selected from C₁₋₄alkyl, C₁₋₄alkoxy, halogen, hydroxy, carboxy, amino, C₁₋₄alkyl-amino or diC₁₋₄alkyl-amino;
V is CH or N;
W is hydrogen or C₁₋₃alkoxy;
R₁ is amino, C₁₋₄alkyl-amino, diC₁₋₄alkyl-amino, hydroxy-amino, amino-C₁₋₄alkyl-carbonyl-amino, C₁₋₄alkyl-amino-C₁₋₄alkyl-carbonyl-amino, diC₁₋₄alkyl-amino-C₁₋₄alkyl-carbonyl-amino, amino-sulfonyl-amino, C₁₋₄alkyl-imino, C₁₋₄alkoxy-imino, hydroxy-imino, amino-imino, C₁₋₄alkyl-amino-imino, diC₁₋₄alkyl-amino-imino, amino-C₁₋₄alkoxy-imino, C₁₋₄alkyl-amino-C₁₋₄alkoxy-imino, diC₁₋₄alkyl-amino-C₁₋₄alkoxy-imino, heteroaryl, heteroaryl-amino-imino or heteroaryl-carbonyl-amino-imino, wherein each heteroaryl is optionally substituted with C₁₋₄alkyl; and,
R₂ is oxo, amino, C₁₋₄alkyl-amino, diC₁₋₄alkyl-amino, hydroxy-amino, amino-C₁₋₄alkyl, C₁₋₄alkyl-amino-C₁₋₄alkyl, diC₁₋₄alkyl-amino-C₁₋₄alkyl, amino-C₁₋₄alkyl-amino, C₁₋₄alkyl-amino-C₁₋₄alkyl-amino, diC₁₋₄alkyl-amino-C₁₋₄alkyl-amino, amino-sulfonyl-amino, amino-sulfonyl-amino-carbonyl, C₁₋₄alkyl-amino-sulfonyl-amino-carbonyl, diC₁₋₄alkyl-amino-sulfonyl-amino-carbonyl, C₁₋₄alkoxy-carbonyl-methylene, carboxy-methylene, amino-carbonyl-methylene, C₁₋₄alkyl-amino-carbonyl-methylene, diC₁₋₄alkyl-amino-carbonyl-methylene, hydroxy-C₁₋₄alkyl-amino-carbonyl-methylene, amino-C₁₋₄alkyl-amino-carbonyl-methylene, C₁₋₄alkyl-amino-C₁₋₄alkyl-amino-carbonyl-methylene, diC₁₋₄alkyl-amino-C₁₋₄alkyl-amino-carbonyl-methylene, heterocyclyl-C₁₋₄alkyl-amino-carbonyl-methylene, C₁₋₄alkyl-imino, C₁₋₄alkoxy-imino, hydroxy-imino, carboxy-C₁₋₄alkoxy-imino, amino-imino, C₁₋₄alkyl-amino-imino, diC₁₋₄alkyl-amino-imino, aryl-oxy-imino, heterocyclyl or heteroaryl, wherein heterocyclyl and heteroaryl are each optionally substituted with C₁₋₄alkyl.

2. The compound of claim 1, wherein
Ring A is selected from the group consisting Ring R₁a, Ring R₁b, Ring R₁c, Ring R₁d, Ring R₁e, Ring R₁f, Ring R₁g, Ring R₁h, Ring R₁i, Ring R₂a, Ring R₂b, Ring R₂c, Ring R₂d, Ring R₂e, Ring R₂f, Ring R₂g, Ring R₂h, and Ring R₂i;
U is phenyl-carbonyl-amino, biphenyl-carbonyl-amino, pyrrolidinyl or pyrazolyl, wherein pyrazolyl is optionally substituted with C₁₋₄alkyl, and wherein each phenyl is optionally substituted with one, two or three substituents independently selected from C₁₋₄alkyl, C₁₋₄alkoxy, halogen, hydroxy, carboxy, amino, C₁₋₄alkyl-amino or diC₁₋₄alkyl-amino;
V is CH or N;
W is hydrogen or C₁₋₃alkoxy;
R₁ is amino, C₁₋₄alkyl-amino, diC₁₋₄alkyl-amino, hydroxy-amino, amino-C₁₋₄alkyl-carbonyl-amino, C₁₋₄alkyl-amino-C₁₋₄alkyl-carbonyl-amino, diC₁₋₄alkyl-amino-C₁₋₄alkyl-carbonyl-amino, amino-sulfonyl-amino, C₁₋₄alkyl-imino, C₁₋₄alkoxy-imino, hydroxy-imino, amino-imino, C₁₋₄alkyl-amino-imino, diC₁₋₄alkyl-amino-imino, amino-C₁₋₄alkoxy-imino, C₁₋₄alkyl-amino-C₁₋₄alkoxy-imino, diC₁₋₄alkyl-amino-C₁₋₄alkoxy-imino, 1H-imidazolyl, pyridinyl-amino-imino or pyridinyl-carbonyl-amino-imino, wherein 1H-imidazolyl is optionally substituted with C₁₋₄alkyl; and,
R₂ is oxo, amino, C₁₋₄alkyl-amino, diC₁₋₄alkyl-amino, hydroxy-amino, amino-C₁₋₄alkyl, C₁₋₄alkyl-amino-C₁₋₄alkyl, diC₁₋₄alkyl-amino-C₁₋₄alkyl, amino-C₁₋₄alkyl-amino, C₁₋₄alkyl-amino-C₁₋₄alkyl-amino, diC₁₋₄alkyl-amino-C₁₋₄alkyl-amino, amino-sulfonyl-amino, amino-sulfonyl-amino-carbonyl, C₁₋₄alkyl-amino-sulfonyl-amino-carbonyl, diC₁₋₄alkyl-amino-sulfonyl-amino-carbonyl, C₁₋₄alkoxy-carbonyl-methylene, carboxy-methylene, amino-carbonyl-methylene, C₁₋₄alkyl-amino-carbonyl-methylene, diC₁₋₄alkyl-amino-carbonyl-methylene, hydroxy-C₁₋₄alkyl-amino-carbonyl-methylene, amino-C₁₋₄alkyl-amino-carbonyl-methylene, C₁₋₄alkyl-amino-C₁₋₄alkyl-amino-carbonyl-methylene, diC₁₋₄alkyl-amino-C₁₋₄alkyl-amino-carbonyl-methylene, morpholinyl-C₁₋₄alkyl-amino-carbonyl-methylene, C₁₋₄alkyl-imino, C₁₋₄alkoxy-imino, hydroxy-imino, carboxy-C₁₋₄alkoxy-imino, amino-imino, C₁₋₄alkyl-amino-imino, diC₁₋₄alkyl-amino-imino, aryl-oxy-imino, pyrrolidinyl, piperazinyl, 4,5-dihydro-1H-imidazolyl, morpholinyl, tetrazolyl or 1H-imidazolyl, wherein piperazinyl and heteroaryl are each optionally substituted with C₁₋₄alkyl.

3. The compound of claim 1, wherein
Ring A is selected from the group consisting Ring R₁a, Ring R₁d, Ring R₁e, Ring R₁f, Ring R₁g, Ring R₂b, Ring R₂e and Ring R₂h;
U is phenyl-carbonyl-amino, biphenyl-carbonyl-amino, heterocyclyl or heteroaryl, wherein heteroaryl is optionally substituted with C₁₋₄alkyl, and wherein each phenyl is optionally substituted with one or two substituents independently selected from C₁₋₄alkyl or halogen;
V is CH or N;
W is hydrogen or C₁₋₃alkoxy;
R₁ is amino, diC₁₋₄alkyl-amino, hydroxy-amino, amino-C₁₋₄alkyl-carbonyl-amino, amino-sulfonyl-amino, C₁₋₄alkoxy-imino, hydroxy-imino, amino-imino, diC₁₋₄alkyl-amino-imino, amino-C₁₋₄alkoxy-imino, heteroaryl, heteroaryl-amino-imino or heteroaryl-carbonyl-amino-imino, wherein each heteroaryl is optionally substituted with C₁₋₄alkyl; and,
R₂ is oxo, amino, diC₁₋₄alkyl-amino, hydroxy-amino, diC₁₋₄alkyl-amino-C₁₋₄alkyl-amino, amino-sulfonyl-amino, amino-sulfonyl-amino-carbonyl, diC₁₋₄alkyl-amino-sulfonyl-amino-carbonyl, C₁₋₄alkoxy-carbonyl-methylene, carboxy-methylene, amino-carbonyl-methylene, C₁₋₄alkyl-amino-carbonyl-methylene, hydroxy-C₁₋₄alkyl-amino-carbonyl-methylene, diC₁₋₄alkyl-amino-C₁₋₄alkyl-amino-carbonyl-methylene, heterocyclyl-C₁₋₄alkyl-amino-carbonyl-methylene, C₁₋₄alkoxy-imino, hydroxy-imino, carboxy-C₁₋₄alkoxy-imino, diC₁₋₄alkyl-amino-imino, heterocyclyl or heteroaryl, wherein heterocyclyl is optionally substituted with C₁₋₄alkyl.

4. The compound of claim 1, wherein
Ring A is selected from the group consisting Ring R₁a, Ring R₁d, Ring R₁e, Ring R₁f, Ring R₁g, Ring R₂b, Ring R₂e and Ring R₂h;
U is phenyl-carbonyl-amino, biphenyl-carbonyl-amino, pyrrolidinyl or pyrazolyl, wherein pyrazolyl is optionally substituted with C₁₋₄alkyl, and wherein each phenyl is optionally substituted with one or two substituents independently selected from C₁₋₄alkyl or halogen;
V is CH or N;
W is hydrogen or C₁₋₃alkoxy;
R₁ is amino, diC₁₋₄alkyl-amino, hydroxy-amino, amino-C₁₋₄alkyl-carbonyl-amino, amino-sulfonyl-amino, C₁₋₄alkoxy-imino, hydroxy-imino, amino-imino, diC₁₋₄alkyl-amino-imino, amino-C₁₋₄alkoxy-imino, 1H-imidazolyl, pyridinyl-amino-imino or pyridinyl-carbonyl-amino-imino, wherein 1H-imidazolyl is optionally substituted with C₁₋₄alkyl; and,
R₂ is oxo, amino, diC₁₋₄alkyl-amino, hydroxy-amino, diC₁₋₄alkyl-amino-C₁₋₄alkyl-amino, amino-sulfonyl-amino, amino-sulfonyl-amino-carbonyl, diC₁₋₄alkyl-amino-sulfonyl-amino-carbonyl, C₁₋₄alkoxy-carbonyl-methylene, carboxy-methylene, amino-carbonyl-methylene, C₁₋₄alkyl-amino-carbonyl-methylene, hydroxy-C₁₋₄alkyl-amino-carbonyl-methylene, diC₁₋₄alkyl-amino-C₁₋₄alkyl-amino-carbonyl-methylene, morpholinyl-C₁₋₄alkyl-amino-carbonyl-methylene, C₁₋₄alkoxy-imino, hydroxy-imino, carboxy-C₁₋₄alkoxy-imino, diC₁₋₄alkyl-amino-imino, pyrrolidinyl, piperazinyl, 4,5-dihydro-1H-imidazolyl, morpholinyl, tetrazolyl or 1H-imidazolyl, wherein piperazinyl is optionally substituted with C₁₋₄alkyl.

5. The compound of claim 1, wherein
Ring A is selected from the group consisting Ring R₁a, Ring R₁d, Ring R₁e, Ring R₁f, Ring R₁g, Ring R₂b, Ring R₂e and Ring R₂h;
U is phenyl-carbonyl-amino, biphenyl-carbonyl-amino, heterocyclyl or heteroaryl, wherein heteroaryl is optionally substituted with C₁₋₄alkyl, and wherein each phenyl is optionally substituted with one or two substituents independently selected from C₁₋₄alkyl or halogen;
V is CH or N;
W is hydrogen or C₁₋₃alkoxy;
R₁ is amino, diC₁₋₄alkyl-amino, hydroxy-amino, amino-C₁₋₄alkyl-carbonyl-amino, amino-sulfonyl-amino, C₁₋₄alkoxy-imino, hydroxy-imino, amino-imino, diC₁₋₄alkyl-amino-imino, amino-C₁₋₄alkoxy-imino, heteroaryl, heteroaryl-amino-imino or heteroaryl-carbonyl-amino-imino, wherein each heteroaryl is optionally substituted with C₁₋₄alkyl; and,
R₂ is diC₁₋₄alkyl-amino, diC₁₋₄alkyl-amino-C₁₋₄alkyl-amino, amino-sulfonyl-amino, amino-sulfonyl-amino-carbonyl, diC₁₋₄alkyl-amino-sulfonyl-amino-carbonyl, C₁₋₄alkoxy-carbonyl-methylene, carboxy-methylene, amino-carbonyl-methylene, C₁₋₄alkyl-amino-carbonyl-methylene, hydroxy-C₁₋₄alkyl-amino-carbonyl-methylene, diC₁₋₄alkyl-amino-C₁₋₄alkyl-amino-carbonyl-methylene, heterocyclyl-C₁₋₄alkyl-amino-carbonyl-methylene, C₁₋₄alkoxy-imino, hydroxy-imino, carboxy-C₁₋₄alkoxy-imino, diC₁₋₄alkyl-amino-imino, heterocyclyl or heteroaryl.

6. The compound of claim 1, wherein
Ring A is selected from the group consisting Ring R₁a, Ring R₁d, Ring R₁e, Ring R₁f, Ring R₁g, Ring R₂b, Ring R₂e and Ring R₂h;
U is phenyl-carbonyl-amino, biphenyl-carbonyl-amino, pyrrolidinyl or pyrazolyl, wherein pyrazolyl is optionally substituted with C₁₋₄alkyl, and wherein each phenyl is optionally substituted with one or two substituents independently selected from C₁₋₄alkyl or halogen;
V is CH or N;
W is hydrogen or C₁₋₃alkoxy;
R₁ is amino, diC₁₋₄alkyl-amino, hydroxy-amino, amino-C₁₋₄alkyl-carbonyl-amino, amino-sulfonyl-amino, C₁₋₄alkoxy-imino, hydroxy-imino, amino-imino, diC₁₋₄alkyl-amino-imino, amino-C₁₋₄alkoxy-imino, 1H-imidazolyl, pyridinyl-amino-imino or pyridinyl-carbonyl-amino-imino, wherein 1H-imidazolyl is optionally substituted with C₁₋₄alkyl; and,
R₂ is diC₁₋₄alkyl-amino, diC₁₋₄alkyl-amino-C₁₋₄alkyl-amino, amino-sulfonyl-amino, amino-sulfonyl-amino-carbonyl, diC₁₋₄alkyl-amino-sulfonyl-amino-carbonyl, C₁₋₄alkoxy-carbonyl-methylene, carboxy-methylene, amino-carbonyl-methylene, C₁₋₄alkyl-amino-carbonyl-methylene, hydroxy-C₁₋₄alkyl-amino-carbonyl-methylene, diC₁₋₄alkyl-amino-C₁₋₄alkyl-amino-carbonyl-methylene, morpholinyl-C₁₋₄alkyl-amino-carbonyl-methylene, C₁₋₄alkoxy-imino, hydroxy-imino, carboxy-C₁₋₄alkoxy-imino, diC₁₋₄alkyl-amino-imino4,5-dihydro-1H-imidazolyl, morpholinyl, tetrazolyl or 1H-imidazolyl.

7. The compound of claim 1, wherein
Ring A is selected from the group consisting Ring R₁a, Ring R₁d, Ring R₁e, Ring R₁g, Ring R₂b, Ring R₂e and Ring R₂h;
U is phenyl-carbonyl-amino or biphenyl-carbonyl-amino, wherein each phenyl is optionally substituted with one or two substituents independently selected from C₁₋₄alkyl or halogen;
V is CH or N;
W is hydrogen or C₁₋₃alkoxy;
R₁ is amino, diC₁₋₄alkyl-amino, hydroxy-amino, amino-C₁₋₄alkyl-carbonyl-amino, amino-sulfonyl-amino, C₁₋₄alkoxy-imino, hydroxy-imino, amino-imino, diC₁₋₄alkyl-amino-imino, amino-C₁₋₄alkoxy-imino, heteroaryl, heteroaryl-amino-imino or heteroaryl-carbonyl-amino-imino, wherein each heteroaryl is optionally substituted with C₁₋₄alkyl; and,
R₂ is amino-sulfonyl-amino, amino-sulfonyl-amino-carbonyl, C₁₋₄alkoxy-carbonyl-methylene, carboxy-methylene, amino-carbonyl-methylene, C₁₋₄alkyl-amino-carbonyl-methylene, hydroxy-C₁₋₄alkyl-amino-carbonyl-methylene, hydroxy-imino or heteroaryl.

8. The compound of claim 1, wherein
Ring A is selected from the group consisting Ring R₁a, Ring R₁d, Ring R₁e, Ring R₁g, Ring R₂b, Ring R₂e and Ring R₂h;
U is phenyl-carbonyl-amino or biphenyl-carbonyl-amino, wherein each phenyl is optionally substituted with one or two substituents independently selected from C₁₋₄alkyl or halogen;
V is CH or N;
W is hydrogen or C₁₋₃alkoxy;
R₁ is amino, diC₁₋₄alkyl-amino, hydroxy-amino, amino-C₁₋₄alkyl-carbonyl-amino, amino-sulfonyl-amino, C₁₋₄alkoxy-imino, hydroxy-imino, amino-imino, diC₁₋₄alkyl-amino-imino, amino-C₁₋₄alkoxy-imino, 1H-imidazolyl, pyridinyl-amino-imino or pyridinyl-carbonyl-amino-imino, wherein 1H-imidazolyl is optionally substituted with C₁₋₄alkyl; and,
R₂ is amino-sulfonyl-amino, amino-sulfonyl-amino-carbonyl, C₁₋₄alkoxy-carbonyl-methylene, carboxy-methylene, amino-carbonyl-methylene, C₁₋₄alkyl-amino-carbonyl-methylene, hydroxy-C₁₋₄alkyl-amino-carbonyl-methylene, hydroxy-imino, tetrazolyl or 1H-imidazolyl.

9. The compound of claim 1, wherein
Ring A is selected from the group consisting Ring R₁d, Ring R₁e and Ring R₁g;
U is phenyl-carbonyl-amino, wherein phenyl is substituted with one or two halogen substituents;
V is CH;
W is hydrogen; and,
R₁ is C₁₋₄alkoxy-imino, hydroxy-imino, amino-imino, diC₁₋₄alkyl-amino-imino, amino-C₁₋₄alkoxy-imino, heteroaryl, or heteroaryl-amino-imino, wherein each heteroaryl is optionally substituted with C₁₋₄alkyl.

10. The compound of claim 1, wherein
Ring A is selected from the group consisting Ring R₁d, Ring R₁e and Ring R₁g;
U is phenyl-carbonyl-amino, wherein phenyl is substituted with one or two halogen substituents;
V is CH;
W is hydrogen; and,
R₁ is C₁₋₄alkoxy-imino, hydroxy-imino, amino-imino, diC₁₋₄alkyl-amino-imino, amino-C₁₋₄alkoxy-imino, 1H-imidazolyl or pyridinyl-amino-imino, wherein 1H-imidazolyl is optionally substituted with C₁₋₄alkyl.

11. The compound of claim 1, wherein Ring A is selected from the group consisting Ring R₁a, Ring R₁d, Ring R₁e, Ring R₁f, Ring R₁g, Ring R₂b, Ring R₂e and Ring R₂h.

12. The compound of claim 1, wherein Ring A is selected from the group consisting Ring R₁a, Ring R₁d, Ring R₁e, Ring R₁g, Ring R₂b, Ring R₂e and Ring R₂h.

13. The compound of claim 1, wherein Ring A is selected from the group consisting Ring R₁d, Ring R₁e and Ring R₁g.

14. The compound of claim 1, wherein U is phenyl-carbonyl-amino, biphenyl-carbonyl-amino, heterocyclyl or heteroaryl, wherein heteroaryl is optionally substituted with C₁₋₄alkyl, and wherein each phenyl is optionally substituted with one or two substituents independently selected from C₁₋₄alkyl or halogen.

15. The compound of claim 1, wherein U is phenyl-carbonyl-amino, biphenyl-carbonyl-amino, pyrrolidinyl or pyrazolyl, wherein pyrazolyl is optionally substituted with C₁₋₄alkyl, and wherein each phenyl is optionally substituted with one or two substituents independently selected from C₁₋₄alkyl or halogen.

16. The compound of claim 1, wherein U is phenyl-carbonyl-amino or biphenyl-carbonyl-amino, and wherein each phenyl is optionally substituted with one or two substituents independently selected from C₁₋₄alkyl or halogen.

17. The compound of claim 1, wherein U is phenyl-carbonyl-amino, and wherein phenyl is substituted with one or two halogen substituents.

18. The compound of claim 1, wherein V is CH.

19. The compound of claim 1, wherein W is hydrogen.

20. The compound of claim 1, wherein R₁ is amino, diC₁₋₄alkyl-amino, hydroxy-amino, amino-C₁₋₄alkyl-carbonyl-amino, amino-sulfonyl-amino, C₁₋₄alkoxy-imino, hydroxy-imino, amino-imino, diC₁₋₄alkyl-amino-imino, amino-C₁₋₄alkoxy-imino, heteroaryl, heteroaryl-amino-imino or heteroaryl-carbonyl-amino-imino, wherein each heteroaryl is optionally substituted with C₁₋₄alkyl.

21. The compound of claim 1, wherein R₁ is amino, diC₁₋₄alkyl-amino, hydroxy-amino, amino-C₁₋₄alkyl-carbonyl-amino, amino-sulfonyl-amino, C₁₋₄alkoxy-imino, hydroxy-imino, amino-imino, diC₁₋₄alkyl-amino-imino, amino-C₁₋₄alkoxy-imino, 1H-imidazolyl, pyridinyl-amino-imino or pyridinyl-carbonyl-amino-imino, wherein 1H-imidazolyl is optionally substituted with C₁₋₄alkyl.

22. The compound of claim 1, wherein R₁ is C₁₋₄alkoxy-imino, hydroxy-imino, amino-imino, diC₁₋₄alkyl-amino-imino, amino-C₁₋₄alkoxy-imino, heteroaryl, or heteroaryl-amino-imino, wherein each heteroaryl is optionally substituted with C₁₋₄alkyl.

23. The compound of claim 1, wherein R₁ is C₁₋₄alkoxy-imino, hydroxy-imino, amino-imino, diC₁₋₄alkyl-amino-imino, amino-C₁₋₄alkoxy-imino, 1H-imidazolyl or pyridinyl-amino-imino, wherein 1H-imidazolyl is optionally substituted with C₁₋₄alkyl.

24. The compound of claim 1, wherein R₂ is oxo, amino, diC₁₋₄alkyl-amino, hydroxy-amino, diC₁₋₄alkyl-amino-C₁₋₄alkyl-amino, amino-sulfonyl-amino, amino-sulfonyl-amino-carbonyl, diC₁₋₄alkyl-amino-sulfonyl-amino-carbonyl, C₁₋₄alkoxy-carbonyl-methylene, carboxy-methylene, amino-carbonyl-methylene, C₁₋₄alkyl-amino-carbonyl-methylene, hydroxy-C₁₋₄alkyl-amino-carbonyl-methylene, diC₁₋₄alkyl-amino-C₁₋₄alkyl-amino-carbonyl-methylene, heterocyclyl-C₁₋₄alkyl-amino-carbonyl-methylene, C₁₋₄alkoxy-imino, hydroxy-imino, carboxy-C₁₋₄alkoxy-imino, diC₁₋₄alkyl-amino-imino, heterocyclyl or heteroaryl, wherein heterocyclyl is optionally substituted with C₁₋₄alkyl.

25. The compound of claim 1, wherein R₂ is oxo, amino, diC₁₋₄alkyl-amino, hydroxy-amino, diC₁₋₄alkyl-amino-C₁₋₄alkyl-amino, amino-sulfonyl-amino, amino-sulfonyl-amino-carbonyl, diC₁₋₄alkyl-amino-sulfonyl-amino-carbonyl, C₁₋₄alkoxy-carbonyl-methylene, carboxy-methylene, amino-carbonyl-methylene, C₁₋₄alkyl-amino-carbonyl-methylene, hydroxy-C₁₋₄alkyl-amino-carbonyl-methylene, diC₁₋₄alkyl-amino-C₁₋₄alkyl-amino-carbonyl-methylene, morpholinyl-C₁₋₄alkyl-amino-carbonyl-methylene, C₁₋₄alkoxy-imino, hydroxy-imino, carboxy-C₁₋₄alkoxy-imino, diC₁₋₄alkyl-amino-imino, pyrrolidinyl, piperazinyl, 4,5-dihydro-1H-imidazolyl, morpholinyl, tetrazolyl or 1H-imidazolyl, wherein piperazinyl is optionally substituted with C₁₋₄alkyl.

26. The compound of claim 1, wherein R₂ is diC₁₋₄alkyl-amino, diC₁₋₄alkyl-amino-C₁₋₄alkyl-amino, amino-sulfonyl-amino, amino-sulfonyl-amino-carbonyl, diC₁₋₄alkyl-amino-sulfonyl-amino-carbonyl, C₁₋₄alkoxy-carbonyl-methylene, carboxy-methylene, amino-carbonyl-methylene, C₁₋₄alkyl-amino-carbonyl-methylene, hydroxy-C₁₋₄alkyl-amino-carbonyl-methylene, diC₁₋₄alkyl-amino-C₁₋₄alkyl-amino-carbonyl-methylene, heterocyclyl-C₁₋₄alkyl-amino-carbonyl-methylene, C₁₋₄alkoxy-imino, hydroxy-imino, carboxy-C₁₋₄alkoxy-imino, diC₁₋₄alkyl-amino-imino, heterocyclyl or heteroaryl.

27. The compound of claim 1, wherein R₂ is diC₁₋₄alkyl-amino, diC₁₋₄alkyl-amino-C₁₋₄alkyl-amino, amino-sulfonyl-amino, amino-sulfonyl-amino-carbonyl, diC₁₋₄alkyl-amino-sulfonyl-amino-carbonyl, C₁₋₄alkoxy-carbonyl-methylene, carboxy-methylene, amino-carbonyl-methylene, C₁₋₄alkyl-amino-carbonyl-methylene, hydroxy-C₁₋₄alkyl-amino-carbonyl-methylene, diC₁₋₄alkyl-amino-C₁₋₄alkyl-amino-carbonyl-methylene, heterocyclyl-C₁₋₄alkyl-amino-carbonyl-methylene, C₁₋₄alkoxy-imino, hydroxy-imino, carboxy-C₁₋₄alkoxy-imino, diC₁₋₄alkyl-amino-imino, 4,5-dihydro-1H-imidazolyl, morpholinyl, tetrazolyl or 1H-imidazolyl.

28. The compound of claim 1, wherein R₂ is amino-sulfonyl-amino, amino-sulfonyl-amino-carbonyl, C₁₋₄alkoxy-carbonyl-methylene, carboxy-methylene, amino-carbonyl-methylene, C₁₋₄alkyl-amino-carbonyl-methylene, hydroxy-C₁₋₄alkyl-amino-carbonyl-methylene, hydroxy-imino or heteroaryl.

29. The compound of claim 1, wherein R₂ is amino-sulfonyl-amino, amino-sulfonyl-amino-carbonyl, C₁₋₄alkoxy-carbonyl-methylene, carboxy-methylene, amino-carbonyl-methylene, C₁₋₄alkyl-amino-carbonyl-methylene, hydroxy-C₁₋₄alkyl-amino-carbonyl-methylene, hydroxy-imino, tetrazolyl or 1H-imidazolyl.

30. A compound selected from the group consisting of:
2-aminoimino-N-[4-(2-chloro-5-fluoro-phenylcarbonyl)amino-phenylcarbonyl]-spiro[cyclopentane-1,4′-benzo[b]azepane],
N-[4-(2-chloro-5-fluoro-phenylcarbonyl)amino-phenylcarbonyl]-2-methoxyimino-spiro[cyclopentane-1,4′-benzo[b]azepane],
N-[4-(2-chloro-5-fluoro-phenylcarbonyl)amino-phenylcarbonyl]-2-dimethylaminoimino-spiro[cyclopentane-1,4′-benzo[b]azepane],
N-[4-(2-chloro-5-fluoro-phenylcarbonyl)amino-phenylcarbonyl]-2-pyridin-3-ylcarbonylaminoimino-spiro[cyclopentane-1,4′-benzo[b]azepane],
N-[4-(2-chloro-5-fluoro-phenylcarbonyl)amino-phenylcarbonyl]-2-(1-methyl-1H-imidazol-2-yl)-spiro[cyclopent-2-ene-1,4′-benzo[b]azepane],
N-[4-(2-chloro-5-fluoro-phenylcarbonyl)amino-phenylcarbonyl]-2-dimethylamino-spiro[cyclopentane-1,4′-benzo[b]azepane],
(R)-N-[4-(2-chloro-5-fluoro-phenylcarbonyl)amino-phenylcarbonyl]-2-hydroxyimino-spiro[cyclopentane-1,4′-benzo[b]azepane],
2-amino-N-[3-methoxy-4-(2-chloro-5-fluoro-phenylcarbonyl)amino-phenylcarbonyl]-spiro[cyclopentane-1,4′-benzo[b]azepane],
N-[3-methoxy-4-(2-chloro-5-fluoro-phenylcarbonyl)amino-phenylcarbonyl]-2-hydroxyimino-spiro[cyclopentane-1,4′-benzo[b]azepane],
N-[6-(2-chloro-5-fluoro-phenylcarbonyl)amino-pyridin-3-ylcarbonyl]-2-hydroxyimino-spiro[cyclopentane-1,4′-benzo[b]azepane],
(R)-N-[3-methoxy-4-(2-chloro-5-fluoro-phenylcarbonyl)amino-phenylcarbonyl]-3-tetrazol-5-yl-spiro[cyclopent-2-ene-1,4′-benzo[b]azepane],
(R)-N-[3-methoxy-4-(2-chloro-5-fluoro-phenylcarbonyl)amino-phenylcarbonyl]-3-(4,5-dihydro-1H-imidazol-2-yl)-spiro[cyclopent-2-ene-1,4′-benzo[b]azepane],
(R)-3-aminosulfonylaminocarbonyl-N-[3-methoxy-4-(2-chloro-5-fluoro-phenylcarbonyl)amino-phenylcarbonyl]-spiro[cyclopent-2-ene-1,4′-benzo[b]azepane],
(R)-N-[3-methoxy-4-(2-chloro-5-fluoro-phenylcarbonyl)amino-phenylcarbonyl]-3-(1H-imidazol-2-yl)-spiro[cyclopent-2-ene-1,4′-benzo[b]azepane],
(R)-N-[3-methoxy-4-(2-chloro-5-fluoro-phenylcarbonyl)amino-phenylcarbonyl]-3-hydroxyimino-spiro[cyclopentane-1,4′-benzo[b]azepane],
(R)-N-[3-methoxy-4-(2-chloro-5-fluoro-phenylcarbonyl)amino-phenylcarbonyl]-3-carboxymethylene-spiro[cyclopentane-1,4′-benzo[b]azepane],
(1R)-3-amino-N-[3-methoxy-4-(2-chloro-5-fluoro-phenylcarbonyl)amino-phenylcarbonyl]-spiro[cyclopentane-1,4′-benzo[b]azepane],
(1R)-N-[3-methoxy-4-(2-chloro-5-fluoro-phenylcarbonyl)amino-phenylcarbonyl]-3-morpholin-4-yl-spiro[cyclopentane-1,4′-benzo[b]azepane],
(1R)-3-aminosulfonylamino-N-[3-methoxy-4-(2-chloro-5-fluoro-phenylcarbonyl)amino-phenylcarbonyl]-spiro[cyclopentane-1,4′-benzo[b]azepane],
(1R)-N-[3-methoxy-4-(2-chloro-5-fluoro-phenylcarbonyl)amino-phenylcarbonyl]-3-dimethylamino-spiro[cyclopentane-1,4′-benzo[b]azepane],
(1R)-N-[3-methoxy-4-(2-chloro-5-fluoro-phenylcarbonyl)amino-phenylcarbonyl]-3-(2-dimethylamino-ethyl)amino-spiro[cyclopentane-1,4′-benzo[b]azepane],
(R)-N-[3-methoxy-4-(2-chloro-5-fluoro-phenylcarbonyl)amino-phenylcarbonyl]-3-methoxyimino-spiro[cyclopentane-1,4′-benzo[b]azepane],
(1R)-N-[3-methoxy-4-(2-chloro-5-fluoro-phenylcarbonyl)amino-phenylcarbonyl]-3-(4-methyl-piperazin-1-yl)-spiro[cyclopentane-1,4′-benzo[b]azepane],
(R)-N-[3-methoxy-4-(2-chloro-5-fluoro-phenylcarbonyl)amino-phenylcarbonyl]-3-carboxymethoxyimino-spiro[cyclopentane-1,4′-benzo[b]azepane],
(1R)-N-[3-methoxy-4-(2-chloro-5-fluoro-phenylcarbonyl)amino-phenylcarbonyl]-3-(1H-pyrrolidin-1-yl)-spiro[cyclopentane-1,4′-benzo[b]azepane],
(R)-N-[3-methoxy-4-(2-chloro-5-fluoro-phenylcarbonyl)amino-phenylcarbonyl]-3-methoxycarbonylmethylene-spiro[cyclopentane-1,4′-benzo[b]azepane],
(R)-N-[3-methoxy-4-(2-chloro-5-fluoro-phenylcarbonyl)amino-phenylcarbonyl]-3-dimethylaminoimino-spiro[cyclopentane-1,4′-benzo[b]azepane],
(R)-N-[3-methoxy-4-(2-chloro-5-fluoro-phenylcarbonyl)amino-phenylcarbonyl]-3-(2-morpholin-4-yl-ethyl)aminocarbonylmethylene-spiro[cyclopentane-1,4′-benzo[b]azepane],
(R)-N-[3-methoxy-4-(2-chloro-5-fluoro-phenylcarbonyl)amino-phenylcarbonyl]-3-(2-hydroxy-ethyl)aminocarbonylmethylene-spiro[cyclopentane-1,4′-benzo[b]azepane],
(R)-N-[3-methoxy-4-(2-chloro-5-fluoro-phenylcarbonyl)amino-phenylcarbonyl]-3-methylaminocarbonylmethylene-spiro[cyclopentane-1,4′-benzo[b]azepane],
(R)-N-[3-methoxy-4-(2-chloro-5-fluoro-phenylcarbonyl)amino-phenylcarbonyl]-3-(2-dimethylamino-ethyl)aminocarbonylmethylene-spiro[cyclopentane-1,4′-benzo[b]azepane],
(R)-3-aminocarbonylmethylene-N-[3-methoxy-4-(2-chloro-5-fluoro-phenylcarbonyl)amino-phenylcarbonyl]-spiro[cyclopentane-1,4′-benzo[b]azepane],
2-aminosulfonylamino-N-[3-methoxy-4-(2-chloro-5-fluoro-phenylcarbonyl)amino-phenylcarbonyl]-spiro[cyclopentane-1,4′-benzo[b]azepane],
(1R,3S)-3-aminosulfonylamino-N-[3-methoxy-4-(2-chloro-5-fluoro-phenylcarbonyl)amino-phenylcarbonyl]-spiro[cyclopentane-1,4′-benzo[b]azepane],
(1R,3R)-3-aminosulfonylamino-N-[3-methoxy-4-(2-chloro-5-fluoro-phenylcarbonyl)amino-phenylcarbonyl]-spiro[cyclopentane-1,4′-benzo[b]azepane],
(R)-N-[3-methoxy-4-(2-chloro-5-fluoro-phenylcarbonyl)amino-phenylcarbonyl]-3-[(dimethylaminosulfonyl)aminocarbonyl]-spiro[cyclopent-2-ene-1,4′-benzo[b]azepane],
N-[4-(biphen-2-ylcarbonyl)amino-phenylcarbonyl]-2-hydroxyimino-spiro[cyclopentane-1,4′-benzo[b]azepane],
2-aminoimino-N-[4-(biphen-2-ylcarbonyl)amino-phenylcarbonyl]-spiro[cyclopentane-1,4′-benzo[b]azepane],
2-amino-N-[4-(biphen-2-ylcarbonyl)amino-phenylcarbonyl]-spiro[cyclopentane-1,4′-benzo[b]azepane],
2-(aminomethylcarbonyl)amino-N-[4-(biphen-2-ylcarbonyl)amino-phenylcarbonyl]-spiro[cyclopentane-1,4′-benzo[b]azepane],
N-[4-(3-fluoro-phenylcarbonyl)amino-phenylcarbonyl]-2-(2-amino-ethoxy)imino-spiro[cyclopentane-1,4′-benzo[b]azepane],
N-[3-methoxy-4-(2-chloro-5-fluoro-phenylcarbonyl)amino-phenylcarbonyl]-2-hydroxyimino-spiro[cyclopentane-1,4′-benzo[b]azepane],
N-[4-(2-chloro-5-fluoro-phenylcarbonyl)amino-phenylcarbonyl]-2-pyridin-2-ylaminoimino-spiro[cyclopentane-1,4′-benzo[b]azepane],
N-[4-(2-chloro-5-fluoro-phenylcarbonyl)amino-phenylcarbonyl]-2-hydroxyimino-spiro[cyclopentane-1,4′-benzo[b]azepane],
(S)-N-[4-(2-chloro-5-fluoro-phenylcarbonyl)amino-phenylcarbonyl]-2-hydroxyimino-spiro[cyclopentane-1,4′-benzo[b]azepane],
N-[4-pyrrolidin-1-yl-phenylcarbonyl]-2-hydroxyimino-spiro[cyclopentane-1,4′-benzo[b]azepane],
N-[4-(3-methyl-1H-pyrazol-1-yl)-phenylcarbonyl]-2-hydroxyimino-spiro[cyclopentane-1,4′-benzo[b]azepane],
N-[4-(2-methyl-5-fluoro-phenylcarbonyl)amino-phenylcarbonyl]-2-hydroxyimino-spiro[cyclopentane-1,4′-benzo[b]azepane],
N-[4-(2-methyl-phenylcarbonyl)amino-phenylcarbonyl]-2-hydroxyimino-spiro[cyclopentane-1,4′-benzo[b]azepane],
2-amino-N-[3-methoxy-4-(3-fluoro-phenylcarbonyl)amino-phenylcarbonyl]-spiro[cyclopentane-1,4′-benzo[b]azepane],
N-[3-methoxy-4-(3-fluoro-phenylcarbonyl)amino-phenylcarbonyl]-2-hydroxyimino-spiro[cyclopentane-1,4′-benzo[b]azepane],
(R)-3-aminosulfonylaminocarbonyl-N-[4-(2-chloro-5-fluoro-phenylcarbonyl)amino-phenylcarbonyl]-spiro[cyclopent-2-ene-1,4′-benzo[b]azepane], and
(R)-N-[3-methoxy-4-(2-chloro-5-fluoro-phenylcarbonyl)amino-phenylcarbonyl]-3-oxo-spiro[cyclopentane-1,4′-benzo[b]azepane].

31. The compound of claim 30, wherein the compound is selected from the group consisting of:
2-aminoimino-N-[4-(2-chloro-5-fluoro-phenylcarbonyl)amino-phenylcarbonyl]-spiro[cyclopentane-1,4′-benzo[b]azepane],
N-[4-(2-chloro-5-fluoro-phenylcarbonyl)amino-phenylcarbonyl]-2-methoxyimino-spiro[cyclopentane-1,4′-benzo[b]azepane],
N-[4-(2-chloro-5-fluoro-phenylcarbonyl)amino-phenylcarbonyl]-2-dimethylaminoimino-spiro[cyclopentane-1,4′-benzo[b]azepane],
N-[4-(2-chloro-5-fluoro-phenylcarbonyl)amino-phenylcarbonyl]-2-pyridin-3-ylcarbonylaminoimino-spiro[cyclopentane-1,4′-benzo[b]azepane],
N-[4-(2-chloro-5-fluoro-phenylcarbonyl)amino-phenylcarbonyl]-2-(1-methyl-1H-imidazol-2-yl)-spiro[cyclopent-2-ene-1,4′-benzo[b]azepane],
N-[4-(2-chloro-5-fluoro-phenylcarbonyl)amino-phenylcarbonyl]-2-dimethylamino-spiro[cyclopentane-1,4′-benzo[b]azepane],
(R)-N-[4-(2-chloro-5-fluoro-phenylcarbonyl)amino-phenylcarbonyl]-2-hydroxyimino-spiro[cyclopentane-1,4′-benzo[b]azepane],
2-amino-N-[3-methoxy-4-(2-chloro-5-fluoro-phenylcarbonyl)amino-phenylcarbonyl]-spiro[cyclopentane-1,4′-benzo[b]azepane],
N-[3-methoxy-4-(2-chloro-5-fluoro-phenylcarbonyl)amino-phenylcarbonyl]-2-hydroxyimino-spiro[cyclopentane-1,4′-benzo[b]azepane],
N-[6-(2-chloro-5-fluoro-phenylcarbonyl)amino-pyridin-3-ylcarbonyl]-2-hydroxyimino-spiro[cyclopentane-1,4′-benzo[b]azepane],
(R)-N-[3-methoxy-4-(2-chloro-5-fluoro-phenylcarbonyl)amino-phenylcarbonyl]-3-tetrazol-5-yl-spiro[cyclopent-2-ene-1,4′-benzo[b]azepane],
(R)-N-[3-methoxy-4-(2-chloro-5-fluoro-phenylcarbonyl)amino-phenylcarbonyl]-3-(4,5-dihydro-1H-imidazol-2-yl)-spiro[cyclopent-2-ene-1,4′-benzo[b]azepane],
(R)-3-aminosulfonylaminocarbonyl-N-[3-methoxy-4-(2-chloro-5-fluoro-phenylcarbonyl)amino-phenylcarbonyl]-spiro[cyclopent-2-ene-1,4′-benzo[b]azepane],
(R)-N-[3-methoxy-4-(2-chloro-5-fluoro-phenylcarbonyl)amino-phenylcarbonyl]-3-(1H-imidazol-2-yl)-spiro[cyclopent-2-ene-1,4′-benzo[b]azepane],
(R)-N-[3-methoxy-4-(2-chloro-5-fluoro-phenylcarbonyl)amino-phenylcarbonyl]-3-hydroxyimino-spiro[cyclopentane-1,4′-benzo[b]azepane],
(R)-N-[3-methoxy-4-(2-chloro-5-fluoro-phenylcarbonyl)amino-phenylcarbonyl]-3-carboxymethylene-spiro[cyclopentane-1,4′-benzo[b]azepane],
(1R)-N-[3-methoxy-4-(2-chloro-5-fluoro-phenylcarbonyl)amino-phenylcarbonyl]-3-morpholin-4-yl-spiro[cyclopentane-1,4′-benzo[b]azepane],
(1R)-3-aminosulfonylamino-N-[3-methoxy-4-(2-chloro-5-fluoro-phenylcarbonyl)amino-phenylcarbonyl]-spiro[cyclopentane-1,4′-benzo[b]azepane],
(1R)-N-[3-methoxy-4-(2-chloro-5-fluoro-phenylcarbonyl)amino-phenylcarbonyl]-3-dimethylamino-spiro[cyclopentane-1,4′-benzo[b]azepane],
(1R)-N-[3-methoxy-4-(2-chloro-5-fluoro-phenylcarbonyl)amino-phenylcarbonyl]-3-(2-dimethylamino-ethyl)amino-spiro[cyclopentane-1,4′-benzo[b]azepane],
(R)-N-[3-methoxy-4-(2-chloro-5-fluoro-phenylcarbonyl)amino-phenylcarbonyl]-3-methoxyimino-spiro[cyclopentane-1,4′-benzo[b]azepane],
(R)-N-[3-methoxy-4-(2-chloro-5-fluoro-phenylcarbonyl)amino-phenylcarbonyl]-3-carboxymethoxyimino-spiro[cyclopentane-1,4′-benzo[b]azepane],
(R)-N-[3-methoxy-4-(2-chloro-5-fluoro-phenylcarbonyl)amino-phenylcarbonyl]-3-methoxycarbonylmethylene-spiro[cyclopentane-1,4′-benzo[b]azepane],
(R)-N-[3-methoxy-4-(2-chloro-5-fluoro-phenylcarbonyl)amino-phenylcarbonyl]-3-dimethylaminoimino-spiro[cyclopentane-1,4′-benzo[b]azepane],
(R)-N-[3-methoxy-4-(2-chloro-5-fluoro-phenylcarbonyl)amino-phenylcarbonyl]-3-(2-morpholin-4-yl-ethyl)aminocarbonylmethylene-spiro[cyclopentane-1,4′-benzo[b]azepane],
(R)-N-[3-methoxy-4-(2-chloro-5-fluoro-phenylcarbonyl)amino-phenylcarbonyl]-3-(2-hydroxy-ethyl)aminocarbonylmethylene-spiro[cyclopentane-1,4′-benzo[b]azepane],
(R)-N-[3-methoxy-4-(2-chloro-5-fluoro-phenylcarbonyl)amino-phenylcarbonyl]-3-methylaminocarbonylmethylene-spiro[cyclopentane-1,4′-benzo[b]azepane],
(R)-N-[3-methoxy-4-(2-chloro-5-fluoro-phenylcarbonyl)amino-phenylcarbonyl]-3-(2-dimethylamino-ethyl)aminocarbonylmethylene-spiro[cyclopentane-1,4′-benzo[b]azepane],
(R)-3-aminocarbonylmethylene-N-[3-methoxy-4-(2-chloro-5-fluoro-phenylcarbonyl)amino-phenylcarbonyl]-spiro[cyclopentane-1,4′-benzo[b]azepane], 2-aminosulfonylamino-N-[3-methoxy-4-(2-chloro-5-fluoro-phenylcarbonyl)amino-phenylcarbonyl]-spiro[cyclopentane-1,4′-benzo[b]azepane],
(1R,3S)-3-aminosulfonylamino-N-[3-methoxy-4-(2-chloro-5-fluoro-phenylcarbonyl)amino-phenylcarbonyl]-spiro[cyclopentane-1,4′-benzo[b]azepane],
(1R,3R)-3-aminosulfonylamino-N-[3-methoxy-4-(2-chloro-5-fluoro-phenylcarbonyl)amino-phenylcarbonyl]-spiro[cyclopentane-1,4′-benzo[b]azepane],
(R)-N-[3-methoxy-4-(2-chloro-5-fluoro-phenylcarbonyl)amino-phenylcarbonyl]-3-[(dimethylaminosulfonyl)aminocarbonyl]-spiro[cyclopent-2-ene-1,4′-benzo[b]azepane],
N-[4-(biphen-2-ylcarbonyl)amino-phenylcarbonyl]-2-hydroxyimino-spiro[cyclopentane-1,4′-benzo[b]azepane],
2-aminoimino-N-[4-(biphen-2-ylcarbonyl)amino-phenylcarbonyl]-spiro[cyclopentane-1,4′-benzo[b]azepane],
2-amino-N-[4-(biphen-2-ylcarbonyl)amino-phenylcarbonyl]-spiro[cyclopentane-1,4′-benzo[b]azepane],
2-(aminomethylcarbonyl)amino-N-[4-(biphen-2-ylcarbonyl)amino-phenylcarbonyl]-spiro[cyclopentane-1,4′-benzo[b]azepane],
N-[4-(3-fluoro-phenylcarbonyl)amino-phenylcarbonyl]-2-(2-amino-ethoxy)imino-spiro[cyclopentane-1,4′-benzo[b]azepane],
N-[3-methoxy-4-(2-chloro-5-fluoro-phenylcarbonyl)amino-phenylcarbonyl]-2-hydroxyimino-spiro[cyclopentane-1,4′-benzo[b]azepane],
N-[4-(2-chloro-5-fluoro-phenylcarbonyl)amino-phenylcarbonyl]-2-pyridin-2-ylaminoimino-spiro[cyclopentane-1,4′-benzo[b]azepane],
N-[4-(2-chloro-5-fluoro-phenylcarbonyl)amino-phenylcarbonyl]-2-hydroxyimino-spiro[cyclopentane-1,4′-benzo[b]azepane],
(S)-N-[4-(2-chloro-5-fluoro-phenylcarbonyl)amino-phenylcarbonyl]-2-hydroxyimino-spiro[cyclopentane-1,4′-benzo[b]azepane],
N-[4-pyrrolidin-1-yl-phenylcarbonyl]-2-hydroxyimino-spiro[cyclopentane-1,4′-benzo[b]azepane],
N-[4-(3-methyl-1H-pyrazol-1-yl)-phenylcarbonyl]-2-hydroxyimino-spiro[cyclopentane-1,4′-benzo[b]azepane],
N-[4-(2-methyl-5-fluoro-phenylcarbonyl)amino-phenylcarbonyl]-2-hydroxyimino-spiro[cyclopentane-1,4′-benzo[b]azepane],
N-[4-(2-methyl-phenylcarbonyl)amino-phenylcarbonyl]-2-hydroxyimino-spiro[cyclopentane-1,4′-benzo[b]azepane],
2-amino-N-[3-methoxy-4-(3-fluoro-phenylcarbonyl)amino-phenylcarbonyl]-spiro[cyclopentane-1,4′-benzo[b]azepane],
N-[3-methoxy-4-(3-fluoro-phenylcarbonyl)amino-phenylcarbonyl]-2-hydroxyimino-spiro[cyclopentane-1,4′-benzo[b]azepane], and
(R)-3-aminosulfonylaminocarbonyl-N-[4-(2-chloro-5-fluoro-phenylcarbonyl)amino-phenylcarbonyl]-spiro[cyclopent-2-ene-1,4′-benzo[b]azepane].

32. The compound of claim 31, wherein the compound is selected from the group consisting of:
2-aminoimino-N-[4-(2-chloro-5-fluoro-phenylcarbonyl)amino-phenylcarbonyl]-spiro[cyclopentane-1,4′-benzo[b]azepane],
N-[4-(2-chloro-5-fluoro-phenylcarbonyl)amino-phenylcarbonyl]-2-methoxyimino-spiro[cyclopentane-1,4′-benzo[b]azepane],
N-[4-(2-chloro-5-fluoro-phenylcarbonyl)amino-phenylcarbonyl]-2-dimethylaminoimino-spiro[cyclopentane-1,4′-benzo[b]azepane],
N-[4-(2-chloro-5-fluoro-phenylcarbonyl)amino-phenylcarbonyl]-2-pyridin-3-ylcarbonylaminoimino-spiro[cyclopentane-1,4′-benzo[b]azepane],
N-[4-(2-chloro-5-fluoro-phenylcarbonyl)amino-phenylcarbonyl]-2-(1-methyl-1H-imidazol-2-yl)-spiro[cyclopent-2-ene-1,4′-benzo[b]azepane],
N-[4-(2-chloro-5-fluoro-phenylcarbonyl)amino-phenylcarbonyl]-2-dimethylamino-spiro[cyclopentane-1,4′-benzo[b]azepane],
(R)-N-[4-(2-chloro-5-fluoro-phenylcarbonyl)amino-phenylcarbonyl]-2-hydroxyimino-spiro[cyclopentane-1,4′-benzo[b]azepane],
2-amino-N-[3-methoxy-4-(2-chloro-5-fluoro-phenylcarbonyl)amino-phenylcarbonyl]-spiro[cyclopentane-1,4′-benzo[b]azepane],
N-[3-methoxy-4-(2-chloro-5-fluoro-phenylcarbonyl)amino-phenylcarbonyl]-2-hydroxyimino-spiro[cyclopentane-1,4′-benzo[b]azepane],
N-[6-(2-chloro-5-fluoro-phenylcarbonyl)amino-pyridin-3-ylcarbonyl]-2-hydroxyimino-spiro[cyclopentane-1,4′-benzo[b]azepane],
(R)-N-[3-methoxy-4-(2-chloro-5-fluoro-phenylcarbonyl)amino-phenylcarbonyl]-3-tetrazol-5-yl-spiro[cyclopent-2-ene-1,4′-benzo[b]azepane],
(R)-3-aminosulfonylaminocarbonyl-N-[3-methoxy-4-(2-chloro-5-fluoro-phenylcarbonyl)amino-phenylcarbonyl]-spiro[cyclopent-2-ene-1,4′-benzo[b]azepane],
(R)-N-[3-methoxy-4-(2-chloro-5-fluoro-phenylcarbonyl)amino-phenylcarbonyl]-3-(1H-imidazol-2-yl)-spiro[cyclopent-2-ene-1,4′-benzo[b]azepane],
(R)-N-[3-methoxy-4-(2-chloro-5-fluoro-phenylcarbonyl)amino-phenylcarbonyl]-3-hydroxyimino-spiro[cyclopentane-1,4′-benzo[b]azepane],
(R)-N-[3-methoxy-4-(2-chloro-5-fluoro-phenylcarbonyl)amino-phenylcarbonyl]-3-carboxymethylene-spiro[cyclopentane-1,4′-benzo[b]azepane],
(R)-N-[3-methoxy-4-(2-chloro-5-fluoro-phenylcarbonyl)amino-phenylcarbonyl]-3-methoxycarbonylmethylene-spiro[cyclopentane-1,4′-benzo[b]azepane],
(R)-N-[3-methoxy-4-(2-chloro-5-fluoro-phenylcarbonyl)amino-phenylcarbonyl]-3-(2-hydroxy-ethyl)aminocarbonylmethylene-spiro[cyclopentane-1,4′-benzo[b]azepane],
(R)-N-[3-methoxy-4-(2-chloro-5-fluoro-phenylcarbonyl)amino-phenylcarbonyl]-3-methylaminocarbonylmethylene-spiro[cyclopentane-1,4′-benzo[b]azepane],
(R)-3-aminocarbonylmethylene-N-[3-methoxy-4-(2-chloro-5-fluoro-phenylcarbonyl)amino-phenylcarbonyl]-spiro[cyclopentane-1,4′-benzo[b]azepane],
2-aminosulfonylamino-N-[3-methoxy-4-(2-chloro-5-fluoro-phenylcarbonyl)amino-phenylcarbonyl]-spiro[cyclopentane-1,4′-benzo[b]azepane],
(1R,3R)-3-aminosulfonylamino-N-[3-methoxy-4-(2-chloro-5-fluoro-phenylcarbonyl)amino-phenylcarbonyl]-spiro[cyclopentane-1,4′-benzo[b]azepane],
N-[4-(biphen-2-ylcarbonyl)amino-phenylcarbonyl]-2-hydroxyimino-spiro[cyclopentane-1,4′-benzo[b]azepane],
2-aminoimino-N-[4-(biphen-2-ylcarbonyl)amino-phenylcarbonyl]-spiro[cyclopentane-1,4′-benzo[b]azepane],
2-amino-N-[4-(biphen-2-ylcarbonyl)amino-phenylcarbonyl]-spiro[cyclopentane-1,4′-benzo[b]azepane],
2-(aminomethylcarbonyl)amino-N-[4-(biphen-2-ylcarbonyl)amino-phenylcarbonyl]-spiro[cyclopentane-1,4′-benzo[b]azepane],
N-[4-(3-fluoro-phenylcarbonyl)amino-phenylcarbonyl]-2-(2-amino-ethoxy)imino-spiro[cyclopentane-1,4′-benzo[b]azepane],
N-[3-methoxy-4-(2-chloro-5-fluoro-phenylcarbonyl)amino-phenylcarbonyl]-2-hydroxyimino-spiro[cyclopentane-1,4′-benzo[b]azepane],
N-[4-(2-chloro-5-fluoro-phenylcarbonyl)amino-phenylcarbonyl]-2-pyridin-2-ylaminoimino-spiro[cyclopentane-1,4′-benzo[b]azepane],
N-[4-(2-chloro-5-fluoro-phenylcarbonyl)amino-phenylcarbonyl]-2-hydroxyimino-spiro[cyclopentane-1,4′-benzo[b]azepane],
N-[4-(2-methyl-5-fluoro-phenylcarbonyl)amino-phenylcarbonyl]-2-hydroxyimino-spiro[cyclopentane-1,4′-benzo[b]azepane],
N-[4-(2-methyl-phenylcarbonyl)amino-phenylcarbonyl]-2-hydroxyimino-spiro[cyclopentane-1,4′-benzo[b]azepane],
N-[3-methoxy-4-(3-fluoro-phenylcarbonyl)amino-phenylcarbonyl]-2-hydroxyimino-spiro[cyclopentane-1,4′-benzo[b]azepane], and
(R)-3-aminosulfonylaminocarbonyl-N-[4-(2-chloro-5-fluoro-phenylcarbonyl)amino-phenylcarbonyl]-spiro[cyclopent-2-ene-1,4′-benzo[b]azepane].

33. The compound of claim 32, wherein the compound is selected from the group consisting of:
2-aminoimino-N-[4-(2-chloro-5-fluoro-phenylcarbonyl)amino-phenylcarbonyl]-spiro[cyclopentane-1,4′-benzo[b]azepane],
N-[4-(2-chloro-5-fluoro-phenylcarbonyl)amino-phenylcarbonyl]-2-methoxyimino-spiro[cyclopentane-1,4′-benzo[b]azepane],
N-[4-(2-chloro-5-fluoro-phenylcarbonyl)amino-phenylcarbonyl]-2-dimethylaminoimino-spiro[cyclopentane-1,4′-benzo[b]azepane],
N-[4-(2-chloro-5-fluoro-phenylcarbonyl)amino-phenylcarbonyl]-2-(1-methyl-1H-imidazol-2-yl)-spiro[cyclopent-2-ene-1,4′-benzo[b]azepane],
(R)-N-[4-(2-chloro-5-fluoro-phenylcarbonyl)amino-phenylcarbonyl]-2-hydroxyimino-spiro[cyclopentane-1,4′-benzo[b]azepane],
N-[4-(3-fluoro-phenylcarbonyl)amino-phenylcarbonyl]-2-(2-amino-ethoxy)imino-spiro[cyclopentane-1,4′-benzo[b]azepane],
N-[4-(2-chloro-5-fluoro-phenylcarbonyl)amino-phenylcarbonyl]-2-pyridin-2-ylaminoimino-spiro[cyclopentane-1,4′-benzo[b]azepane], and
N-[4-(2-chloro-5-fluoro-phenylcarbonyl)amino-phenylcarbonyl]-2-hydroxyimino-spiro[cyclopentane-1,4′-benzo[b]azepane].

34. The compound of claim 1, wherein the compound is an isolated form.

35. A pharmaceutical composition comprising at least one compound of claim 1, in combination with at least one pharmaceutically acceptable carrier or excipient.

36. The pharmaceutical composition of claim 35, wherein the composition comprises at least one compound of claim 30.

37. A process for making a pharmaceutical composition comprising mixing a compound of claim 1 and a pharmaceutically acceptable carrier.

38. A method for treating a vasopressin receptor mediated condition in a patient in need thereof comprising the step of administering to the patient an effective amount of the compound of claim 1.

39. The method of claim 38, wherein the vasopressin receptor is the vasopressin 1 a receptor, the vasopressin 2 receptor or both the vasopressin 1a and vasopressin 2 receptors.

40. The method of claim 38, wherein the vasopressin receptor mediated condition is selected from edema, ischemia, inner ear disorders, hypertension, congestive heart failure, cardiac insufficiency, hyponatremia, coronary vasospasm, cardiac ischemia, liver cirrhosis, renal vasospasm, renal failure, polycystic kidney disease, diabetic nephropathy, cerebral edema and ischemia, stroke, thrombosis, water retention, aggression, obsessive-compulsive disorders, dysmenorrhea, nephrotic syndrome, anxiety and central nervous injuries.

41. The method of claim 40, wherein the condition is selected from hypertension, congestive heart failure, cardiac insufficiency, diabetic nephropathy, dysmenorrhea, renal failure, hyponatremia or stroke.

42. The method of claim 40, wherein the condition is selected from congestive heart failure, diabetic nephropathy, dysmenorrhea, renal failure or hyponatremia.

43. The method of claim 40, wherein the effective amount is from about 0.001 mg/kg/day to about 300 mg/kg/day.

44. A use of the compound of claim 1 in the manufacture of a medicament for treating a vasopressin receptor mediated condition.

45. The use of claim 44, wherein the vasopressin receptor mediated condition is selected from edema, ischemia, inner ear disorders, hypertension, congestive heart failure, cardiac insufficiency, hyponatremia, coronary vasospasm, cardiac ischemia, liver cirrhosis, renal vasospasm, renal failure, polycystic kidney disease, diabetic nephropathy, cerebral edema and ischemia, stroke, thrombosis, water retention, aggression, obsessive-compulsive disorders, dysmenorrhea, nephrotic syndrome, anxiety and central nervous injuries.

46. A use of the compound of claim 1 as a medicine.
